(19) United States
(12) Patent Application Publication
(10) Pub. No.: US 2022/0188654 A1
(43) Pub. Date: Jun. 16, 2022

(54) SYSTEM AND METHOD FOR CLINICAL TRIAL ANALYSIS AND PREDICTIONS USING MACHINE LEARNING AND EDGE COMPUTING

(71) Applicant: Ro5 Inc, Dallas, TX (US)

(72) Inventors: Charles Dazler Knuff, Dallas, TX (US); Roy Tal, Dallas, TX (US); Zygimantas Jocys, Hove (GB); Danius Jean Backis, Vilnius (LT); Artem Krasnoslobodtsev, Frisco, TX (US)

(21) Appl. No.: 17/313,831

(22) Filed: May 6, 2021

Related U.S. Application Data
(63) Continuation-in-part of application No. 17/237,458, filed on Apr. 22, 2021, now Pat. No. 11,256,995.
Application No. 17/237,458 is a continuation-in-part of application No. 17/202,722, filed on Mar. 16, 2021, now Pat. No. 11,256,994.
Application No. 17/202,722 is a continuation-in-part of application No. 17/174,677, filed on Feb. 12, 2021.
Application No. 17/174,677 is a continuation of application No. 17/171,494, filed on Feb. 9, 2021, now Pat. No. 11,176,462.
Application No. 17/171,494 is a continuation of application No. 17/166,435, filed on Feb. 3, 2021, now Pat. No. 11,080,607.
Application No. 17/166,435 is a continuation-in-part of application No. 17/177,565, filed on Feb. 17, 2021, now Pat. No. 11,257,594.
Application No. 17/177,565 is a continuation-in-part of application No. 17/175,832, filed on Feb. 15, 2021.
(60) Provisional application No. 63/126,349, filed on Dec. 16, 2020.
(60) Provisional application No. 63/126,372, filed on Dec. 16, 2020.
(60) Provisional application No. 63/126,388, filed on Dec. 16, 2020.
(60) Provisional application No. 63/135,892, filed on Jan. 11, 2021.
(60) Provisional application No. 63/136,556, filed on Jan. 12, 2021.

(51) Int. Cl. G06N 5/02; G06K 9/62; G06F 16/951; G06N 3/08

(52) U.S. Cl. CPC G06N 5/022; G06N 3/08; G06F 16/951; G06K 9/6215

(57) ABSTRACT

A system and method for improving the efficiency of information flow of and during clinical trials and also using edge-based and cloud-based machine learning for analyzing clinical trial data from inception to completion subsequently protecting investments, assets, and human life. The system comprises a pharmaceutical research system that receives, pushes, and facilitates data packets containing clinical trial information across multiple sites and across multiple trial personnel while also using machine learning for a variety of tasks. A mobile application on edge devices uses edge-based machine learning to identify biomarkers and provides sponsors and clinicians with an expedient and secure communication means. The edge devices and the cloud-based machine learning communicate full-duplex and share information and machine learning models leading to an improvement in early adverse effects detection. Biomarkers predicting severe adverse effects trigger the system to send alerts, reports, and potential victims to medical personnel for immediate intervention.

### CROSS-REFERENCE TO RELATED APPLICATIONS

Priority is claimed in the application data sheet to the following patents or patent applications, the entire written description of each of which is expressly incorporated herein by reference in its entirety:

Ser. No. 17/237,458

Ser. No. 17/202,722

Ser. No. 17/174,677

Ser. No. 63/126,388

Ser. No. 17/171,494

Ser. No. 63/126,372

Ser. No. 17/166,435

Ser. No. 63/126,349

Ser. No. 17/177,565

Ser. No. 63/136,556

Ser. No. 17/175,832

Ser. No. 63/135,892

### BACKGROUND

### Field of the Art

The disclosure relates to the field of medical research, and more particularly to the field of clinical trials, and information processing and analysis.

### Discussion of the State of the Art

Clinical trials are a paramount step in the process for getting potential drugs to market. The clinical trials are used to determine whether new drugs are both safe and effective. Currently, this process has a significant time-cost due to many factors; one major factor is recognizing the safety and efficacy of the drug over time by analyzing and comparing biomarkers between control groups. What makes this factor and others so time-costly, is that trial sites are geographically diverse, and the flow of information and the subsequent analysis of that information from those sites is equally diverse in standards and formats and rely on human organization and interpretation.

Furthermore, some recent trials have had unacceptable death rates due to a lack of early prediction, detection, and recognition of severe adverse effects. In other words, the current inept flow and analysis of trial site information makes ready the need for a new information flow and analysis solution. Predicting and recognizing the early indications of adverse effects for a potential new drug and identifying the at-risk patients before an illness or untimely death strikes is both imperative and optimal.

What is needed is a system and method for clinical trial information transmission, processing, and analysis for the improvement of clinical trial procedures, results, and the protection of human life.

### SUMMARY

Accordingly, the inventor has conceived and reduced to practice, a system and method for improving the efficiency of information flow of and during clinical trials and also using edge-based and cloud-based machine learning for analyzing clinical trial data from inception to completion subsequently protecting investments, assets, and human life. The system comprises a pharmaceutical research system that receives, pushes, and facilitates data packets containing clinical trial information across multiple sites and across multiple trial personnel while also using machine learning for a variety of tasks. A mobile application on edge devices uses edge-based machine learning to identify biomarkers and provides sponsors and clinicians with an expedient and secure communication means. The edge devices and the cloud-based machine learning communicate full-duplex and share information and machine learning models leading to an improvement in early adverse effects detection. Biomarkers predicting severe adverse effects trigger the system to send alerts, reports, and potential victims to medical personnel for immediate intervention.

According to a first preferred embodiment, a system for clinical trial communications, analysis, and predictions is disclosed, comprising: a software application running on an edge device; a computer system comprising a memory and a processor; a clinical trials module, comprising a first plurality of programming instructions stored in the memory and operating on the processor, wherein the first plurality of programming instructions, when operating on the processor, causes the computer system to: receive clinical trial parameters, wherein the clinical trial parameters comprise target biomarkers and trial endpoints; train a machine learning model according to the clinical trial parameters; train the machine learning model to also identify additional biomarkers; train the machine learning model to also predict adverse effects in a trial patient; deploy the machine learning model to the software application; receive patient data from the software application; use the patient data to iterate training of the machine learning model, each iteration creating an updated machine learning model; deploy the updated machine learning model to the software application; issue an alert to the software application if one of the adverse effects is predicted in at least one of the trial patients, the alert comprising identifying information of all the patients at risk of the predicted adverse effects; generate a detailed report about the predicted adverse effects and the biomarkers associated with the predicted adverse effects; and send the generated detailed report to the software application.

According to a second preferred embodiment, a method for clinical trial communications, analysis, and predictions is disclosed, comprising the steps of: receiving clinical trial parameters, wherein the clinical trial parameters comprise target biomarkers and trial endpoints; training a machine learning model according to the clinical trial parameters; training the machine learning model to also identify additional biomarkers; training the machine learning model to also predict adverse effects in a trial patient; deploying the machine learning model to the software application; receiving patient data from the software application; using the patient data to iterate training of the machine learning model, each iteration creating an updated machine learning model; deploying the updated machine learning model to the software application; issuing an alert to the software application if one of the adverse effects is predicted in at least one of the trial patients, the alert comprising identifying information of all the patients at risk of the predicted adverse effects; generating a detailed report about the predicted adverse effects and the biomarkers associated with the predicted adverse effects; and sending the generated detailed report to the software application.

According to various aspects; wherein adverse effects comprise one or both adverse effects and severe adverse effects; wherein the patient data comprises data selected from the group consisting of biometrics, biomarkers, medical history, and vital signs; wherein the clinical trials module receives preclinical trial data; wherein the preclinical trial data is used by machine learning to determine target patient groups for a clinical trial; wherein the preclinical trial data is comparatively analyzed by machine learning against past and current clinical trials; wherein the analytical comparison is used to generate a report of the analytical comparison to the software application; wherein the generated analytical comparison report is sent to the software application; wherein the alert triggers a notification on the edge device; wherein the machine learning model training is supplemented by data from one or more of the modules selected from the group consisting of a bioactivity module, a de novo ligand discovery module, and an ADMET module.

### BRIEF DESCRIPTION OF THE DRAWING FIGURES

The accompanying drawings illustrate several aspects and, together with the description, serve to explain the principles of the invention according to the aspects. It will be appreciated by one skilled in the art that the particular arrangements illustrated in the drawings are merely exemplary, and are not to be considered as limiting of the scope of the invention or the claims herein in any way.

FIG. 1 is a block diagram illustrating an exemplary overall system architecture for a pharmaceutical research system.

FIG. 2 is a block diagram illustrating an exemplary system architecture for an embodiment of a pharmaceutical research system utilizing combined graph-based and sequence-based prediction of molecule bioactivity.

FIG. 3 is a relational diagram illustrating several types of information that may be included in a knowledge graph for a pharmaceutical research system and exemplary relations between those types of information.

FIG. 4 is a diagram illustrating the conceptual layering of different types of information in a knowledge graph.

FIG. 5 is a relational diagram illustrating the use of a knowledge graph to predict usefulness of a molecule in treating a disease.

FIG. 6 is a diagram illustrating an exemplary process for combining various types of information into a knowledge graph suitable for a pharmaceutical research system.

FIG. 7 is a diagram illustrating an exemplary graph-based representation of molecules as simple relationships between atoms using a matrix of adjacencies.

FIG. 8 is a diagram illustrating an exemplary graph-based representation of molecules as relationships between atoms using a matrix of adjacencies wherein the type bonds are distinguished.

FIG. 9 is a diagram illustrating an exemplary graph-based representation of molecules as relationships between atoms using a matrix of adjacencies using SMILES string encoding and one-hot vectors indicating the types of bonds between atoms.

FIG. 10 is a diagram illustrating an exemplary architecture for prediction of molecule bioactivity using concatenation of outputs from a graph-based neural network which analyzes molecule structure and a sequence-based neural network which analyzes protein structure.

FIGS. 11A and 11B illustrates an exemplary implementation of an architecture for prediction of molecule bioactivity using concatenation of outputs from a graph-based neural network which analyzes molecule structure and a sequence-based neural network which analyzes protein structure.

FIG. 12 illustrates an exemplary implementation of the molecule attention assignment aspect of an architecture for prediction of molecule bioactivity using concatenation of outputs from a graph-based neural network which analyzes molecule structure and a sequence-based neural network which analyzes protein structure.

FIG. 13 is a diagram illustrating an exemplary architecture for prediction of molecule bioactivity using concatenation of outputs from a graph-based neural network and an attention-based transformer.

FIG. 14 is a flow diagram illustrating an exemplary method for active example generation.

FIG. 15 is a flow diagram illustrating an exemplary method for active example generation using a graph-based approach.

FIG. 16 is a flow diagram illustrating an exemplary method for active example generation using a 3D CNN approach.

FIG. 17 is a diagram illustrating the training of an autoencoder of a 3D CNN for active example generation.

FIG. 18 is a diagram illustrating the interfacing of the decoder to the 3D-CNN bioactivity prediction model.

FIG. 19 is a diagram illustrating molecule encodings in latent space.

FIG. 20 is a block diagram of an overall model architecture of a system for de novo drug discovery according to one embodiment.

FIG. 21 is a block diagram of a model architecture of a MPNN encoder for de novo drug discovery according to one embodiment.

FIG. 22 is a block diagram of a model architecture of a Sampling module for de novo drug discovery according to one embodiment.

FIG. 23 is a block diagram of a model architecture of a decoder for de novo drug discovery according to one embodiment.

FIG. 24 is a block diagram of a model architecture for reinforcement learning for de novo drug discovery according to one embodiment.

FIG. 25 is a block diagram of a model architecture of an autoregressive decoder for de novo drug discovery according to one embodiment.

FIG. 26 is a block diagram of an exemplary system architecture for a 3D Bioactivity platform.

FIG. 27 is a block diagram of an exemplary model architecture for a 3D Bioactivity platform.

FIG. 28 is a flow diagram illustrating an exemplary method for classifying protein-ligand pairs using a 3D Bioactivity platform.

FIG. 29 is a flow diagram illustrating an exemplary method for generating data for use in training a 3D-CNN used by a 3D Bioactivity platform.

FIG. 30 is a block diagram of an exemplary system architecture for a Point-Cloud Bioactivity platform.

FIG. 31 is a block diagram of an exemplary model architecture for a Point-Cloud Bioactivity platform.

FIG. 32 is a flow diagram illustrating an exemplary method for classifying protein-ligand pairs using a Point-Cloud Bioactivity platform.

FIG. 33 is a block diagram illustrating an exemplary point-based visualization.

FIG. 34 is a diagram of exemplary pseudocode for implementing a point-cloud based machine learning architecture.

FIG. 35 is a block diagram illustrating an exemplary system architecture for biomarker-outcome prediction and clinical trial exploration, according to an embodiment.

FIG. 36 is a flow diagram illustrating an exemplary method for calculating the association score for a biomarker-outcome bigram, according to one embodiment.

FIG. 37 is a diagram illustrating an exemplary interactive exploration tool, according to an embodiment.

FIG. 38 is a diagram illustrating an exemplary output list generated from an input list of biomarkers to be measured, according to an embodiment.

FIG. 39 is a block diagram illustrating an exemplary system architecture for biomarker-outcome prediction and clinical trial exploration for edge computing.

FIG. 40 is a block diagram illustrating an exemplary overall system architecture for a pharmaceutical research system for edge computing.

FIG. 41 is a block diagram illustrating an exemplary system architecture for an embodiment of a pharmaceutical research system utilizing edge devices.

FIG. 42 is a flow diagram illustrating an exemplary method for clinical trial analysis using a pharmaceutical research system.

FIG. 43 illustrates the inputs and outputs of a machine learning model for clinical trial analysis.

FIG. 44 is a diagram of exemplary services provided to edge devices.

FIG. 45 is a block diagram illustrating an exemplary hardware architecture of a computing device.

FIG. 46 is a block diagram illustrating an exemplary logical architecture for a client device.

FIG. 47 is a block diagram showing an exemplary architectural arrangement of clients, servers, and external services.

FIG. 48 is another block diagram illustrating an exemplary hardware architecture of a computing device.

### DETAILED DESCRIPTION

Accordingly, the inventor has conceived and reduced to practice, a system and method for improving the efficiency of information flow of and during clinical trials and also using edge-based and cloud-based machine learning for analyzing clinical trial data from inception to completion subsequently protecting investments, assets, and human life. The system comprises a pharmaceutical research system that receives, pushes, and facilitates data packets containing clinical trial information across multiple sites and across multiple trial personnel while also using machine learning for a variety of tasks. A mobile application on edge devices uses edge-based machine learning to identify biomarkers and provides sponsors and clinicians with an expedient and secure communication means. The edge devices and the cloud-based machine learning communicate full-duplex and share information and machine learning models leading to an improvement in early adverse effects detection. Biomarkers predicting severe adverse effects trigger the system to send alerts, reports, and potential victims to medical personnel for immediate intervention.

The system takes in biomarkers from a variety of sources (e.g., IoT devices, smart wearables, notes and biometrics (i.e., vital signs, etc.) entered by medical personnel, Internet-connected medical devices, i.e., glucose meters, heart monitors, etc., and lab results, i.e., blood, urine, etc.) and analyzes them in real-time looking for indications of sever adverse effects or adverse effects.

For example, consider a clinical trial with many geographically disperse sites. Each site having 30 or more patients. Imagine further that in only one site, a patient's blood results have high levels of brain natriuretic peptides, an indication the heart is not working as it should. The clinician on the ground at that site may not flag that as a concern. But now, one or two patients at some other sites now have the same blood test results. What needs to happen is that all the sites with these anomalous blood sample readings need to share that information, and then decide along with the clinical trial sponsors whether it poses a grave enough threat to remove those patients from the trial or wait to see what happens. However, during this deliberation those patients may have already died. Or maybe the trial orchestrators decided to wait, not fully understanding the implications and indications, and the patients died anyhow. This in fact has happened in the real world and is devastating to all involved.

The various embodiments disclosed herein would allow that all patients would share their biomarkers seamlessly with the Sponsor or CRO which is in charge of the trial. This, in turn, would allow the Sponsor/CRO to adjust their required sample size based on the treatment effect at any point in time, or to withdraw patients faster in case of emergencies. This would work and would be extremely useful for three reasons.

The first reason is that the enrollment of patients is done continuously. This means that not all patients enroll during the same day, therefore some estimates can be drawn from the first 10% or 20% of the patients.

The second reason being that the primary/secondary endpoints of clinical trials are usually some measurable biomarkers. The statistical significance of the difference in treatment effect is calculated by getting the changes of biomarkers from the start of the clinical trial up to a time T. Therefore, having access to the primary endpoint value at any time T would allow to compute the statistical significance at this time T. Then, what would be left to do would be to use the actual difference in treatment effect at this time T to estimate the sample size needed to achieve a 5% or 1% statistical significance.

Third, to get access to all the data, sponsors/CROs have to keep in touch with tens or hundreds of clinical sites at the same time. Because sites do not necessarily communicate between them, it is harder to detect an outlier patient who is at risk. If they used this tool, this would allow them to get access to all the biomarkers plus self-reported AEs (that must be reported to the sponsor) much faster.

Now imagine a second example, the same trial and number of sites and patients. A few patients also having high brain natriuretic peptides levels. However, in this example, the claimed invention is receiving those blood analyses in real-time, comparing those patients and the patient's other biometrics (i.e., vital signs, etc.) data with the rest of the patients for differences and commonalities. The system also compares the current trial with past and other ongoing clinical trials. The machine learning aspect of the system arriving at a higher confidence decision to remove those patients and potentially other patients who share similar traits and biomarkers faster than it took the sites in the first example to share information, thus better protecting human life and producing successful trials.

The machine learning aspect involves edge devices (smart phones, tablets, laptops, IoT devices, etc.) and a cloud-based system. The edge devices run machine learning models such as classifiers that can inform clinicians of such SAEs and AEs like the examples above. The cloud-based machine learning trains an overall model for predictions and detections, while also training the edge device classifier. The cloud-based aspect pushing updated models periodically to the edge devices. Some edge devices may be able to perform model training on their own, and is anticipated in various embodiments.

According to one embodiment, a system and method for biomarker-outcome prediction and medical literature exploration is disclosed which utilizes a data platform to analyze, optimize, and explore the knowledge contained in or derived from clinical trials. The system utilizes a knowledge graph and data analysis engine capabilities of the data platform. The knowledge graph may be used to link biomarkers with molecules, proteins, and genetic data to provide insight into the relationship between biomarkers, outcomes, and adverse events. The system uses natural language processing techniques on a large corpus of medical literature to perform advanced text mining to identify biomarkers associated with adverse events and to curate a comprehensive profile of biomarker-outcome associations. These associations may then be ranked to identify the most-common biomarker-outcome association pairs. Having a comprehensive profile of ranked biomarker-outcome data allows the system to predict biomarkers associated with a given disease and serious adverse events linked to biomarker data.

Cases of fabrication or falsification of data in clinical trials occur sometimes and it is highly plausible that there are additional undetected or unreported cases. The adoption of better clinical trial monitoring procedures can identify potential data fraud not detected by conventional on-site monitoring and might improve overall data quality. According to various embodiments contained herein, a means to allow to distinguish incorrect dates, under-reporting of adverse events, integer rounding of biomarker values, digit preference, extreme variances and unusual correlation structures to detect data fraud that sometimes appears at the clinical site level is disclosed.

A large plurality of biomarkers-outcomes associations are observed empirically and are publicly available through a quick internet search. A few examples of such associations are high cholesterol-high blood pressure, high cholesterol-heart failure, or elevated glucose level-chronic constipation. However, there is still a vast amount of biomarker-outcome associations buried in the biomedical literature. The clinical trial prediction and exploration system may leverage the massive corpus of pharmaceutical information, particularly data extracted from biomedical literature, and implement an automated text mining tool to curate biomarker-outcome associations and parse clinical trials into a data format that allows for easy exploration of historical clinical trial data.

The data platform may utilize a natural language processing (NLP) based automated text mining tool which scrapes medical literature (e.g., clinical trial, assay, and research publications) to populate the data fields of a standard clinical trial data model. The standard clinical trial data model may include data fields for clinical trial information including, but not limited to, publication title, geolocation data identifying the research center or institute which conducted the clinical trial, the trial phase in which the clinical trial ended, date of publication, a link to the original publication, biomarkers studied or identified during research, outcomes predicted and observed, population sample size, population demographics, and medical intervention of interest such as pharmaceutical drug or treatment process. Most clinical trials are regulated and standardized via a set of rules and procedures defined by some regulatory or governmental organization, for instance, the Federal Drug Administration. As a result, a standard clinical trial data model may be developed and utilized by the system to organize the information contained within clinical trial publications and to facilitate easier exploration of historical clinical trial data via a knowledge graph. A clinical trial may be scraped and a standard data model of the clinical trial is generated which is then persisted to a knowledge graph which may be traversed to explore all available clinical trial data. For example, the system may be queried to provide all clinical trials conducted and published by a specified research center. Similarly, the system may be queried to identify all outcomes associated with one or more specified biomarkers. The standard clinical trial data model may also allow for exploration of historical clinical trial data using one or more of the data fields, for example the system may be queried to return all clinical trials published in a given year, or range of years.

The clinical trial prediction and exploration system may allow a client (pharmaceutical company, contract research organization, etc.), who is interested in running a clinical trial, to input a list of biomarkers that will be measured during screening or continuously throughout a clinical trial for each patient. The system, utilizing the automated text mining tool, may return for each biomarker a list of papers that contain associations between that biomarker and side effects, diseases, adverse events, etc. In addition to the list of papers, the system may calculate and return an association score between a biomarker and some outcome. The association score may be derived from calculating the co-occurrence of a biomarker and some outcome across all available medical literature using an automated text mining tool. Ranking biomarker-outcome associations allows the system to link biomarkers to serious adverse events which provides a new and useful tool for developing and analyzing genetic profiles of patients. Furthermore, ranking biomarker-outcome associations allows the system to suggest adverse events that may be predicted from the biomarker data.

Edge ML would provide a substantial advantage here as well: because all the models, their weights, the links between biomarkers and SAEs would be stored on the edge device (while most of the training will be done on the cloud), the patient would not necessarily need to connect to a cloud-based system in order to get a prediction or to be informed that he or she is at risk. In other words, because most of the analysis would be done before-hand, the patients may upload their lab measurements and SAEs to the edge device to get predictions or useful information without needing to connect to a wireless network. That would be especially important in regions our countries where the connection is slower or unstable. Of course, once the patient would have access to an internet connection, the synchronization of the data would be made automatically.

By combining a standard data model with the linked and ranked biomarker-adverse event associations the system may facilitate demographic based queries to attain new insights derived from historical clinical trial data. For example, the system may receive as input a list of biomarkers such as chronic heart failure, Caucasian (a subset of population), and cholesterol and return a list of papers that provide relevant information for that subset of the population in the context of the other biomarkers. In an embodiment, to help with clinical trial design the system may identify at-risk populations based on biomarkers and trial drug characteristics. For instance, a biomarker may be associated with some biological process and the biological process may be regulated by certain proteins and furthermore, the protein function may be impacted by some molecule which may be present in a drug. Using the data platform knowledge graph the system may be able quickly identify the connection, via biological pathways, between a biomarker and a trial drug. At-risk populations may be selected based upon identified biological pathways that may be compromised due to underlying conditions, genetics, physical disposition, etc. For example, a population with low blood pressure biomarkers could be considered an at-risk population for a drug that purports to lower blood pressure to cause some effect.

One of the key aspects of planning a useful and meaningful clinical trial is sample size estimation. Underestimation of sample size may result in a drug turning out to be statistically non-significant even though clinical significance exists. Over estimation of sample size may lead to other issues that should be considered: a smaller sample size may have been used to prove statistical significance which can raise ethical issues as more test subjects were exposed to the test drug, which could have deserved trialing the new drug being researched, and a large sample size may mean even a small difference between the trial drug and the test drug will turn out statistically significant even if that difference is not clinically meaningful. Therefore, sample size is an important factor for approval or rejection of clinical trial results regardless of how clinically effective or ineffective the test drug may be. Typical sample size estimation depends on a few basic requirements including, but not limited to, Types I and II error and Power, study design (e.g., parallel group, crossover group, etc.), study endpoint and its description (e.g., discrete, time-to-event, continuous, etc.), expected response test versus control, clinical meaningful margin which defines the difference between test and reference which can be considered clinically meaningful, level of significance (typically value is 5% or less), and participant drop-out rate. The clinical trial prediction and exploration system may feature a sample size calculator with the prior information sampled from the historical clinical trial data based on locations of the previous trials. In one embodiment, a system user may input singly or in some combination a biomarker, drug, disease, and information about a potential clinical trial such as the study design, a trial drug, and an endpoint and the system will retrieve historical clinical trial data related to and associated with the input and then analyze the data to estimate a sample size appropriate for producing statistically meaningful results.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Definitions

“Bioactivity” as used herein means the physiological effects of a molecule on an organism (i.e., living organism, biological matter).

“Biomarker” as used herein refers to anything that can be used as an indicator of a particular disease state or some other physiological state of a person. Biomarkers can be characteristic biological properties or molecules that can be detected and measured in parts of the body like the blood or tissue. For example, biomarkers may include, but are not limited to, high-cholesterol, blood pressure, specific cells, molecules, genes, gene products, enzymes, hormones, complex organ function, and general characteristic changes in biological structures.

“Docking” as used herein means a method which predicts the orientation of one molecule to a second when bound to each other to form a stable complex. Knowledge of the preferred orientation in turn may be used to predict the strength of association or binding affinity between two molecules.

“Edge device” as used herein means a computing system which is part of a distributed computing topology in which information processing is located close to the edge—where things and people produce or consume that information. Edge devices are equipment deployed at the end of the network that deliver the computing services and process information for that location. Edge devices may include, but are not limited to, smartphones, Internet-of-Things devices, sensors, laptops, desktops, microcontrollers, field-programmable gate arrays, home automation devices, operation technology devices, etc.

“Edges” as used herein means connections between nodes or vertices in a data structure. In graphs, an arbitrary number of edges may be assigned to any node or vertex, each edge representing a relationship to itself or any other node or vertex. Edges may also comprise value, conditions, or other information, such as edge weights or probabilities.

“FASTA” as used herein means any version of the FASTA family (e.g., FASTA, FASTP, FASTA, etc.) of chemical notations for describing nucleotide sequences or amino acid (protein) sequences using text (e.g., ASCII) strings.

“Force field” as used herein means a collection of equations and associated constants designed to reproduce molecular geometry and selected properties of tested structures. In molecular dynamics a molecule is described as a series of charged points (atoms) linked by springs (bonds).

“Ligand” as used herein means a substance that forms a complex with a biomolecule to serve a biological purpose. In protein-ligand binding, the ligand is usually a molecule which produces a signal by binding to a site on a target protein. Ligand binding to a receptor protein alters the conformation by affecting the three-dimensional shape orientation. The conformation of a receptor protein composes the functional state. Ligands comprise substrates, inhibitors, activators, signaling lipids, and neurotransmitters.

“Mobile app” as used herein is an abbreviated version of “mobile application” and means any software designed to run on a computer system, particularly an edge device. While desktop and server computing systems are typically not mobile, the mobile app described herein may run on any such computing system whether the computing system is designed to be mobile or not. A mobile app may be a native application, wherein a native application is created for each specific computing platform. A mobile application may be a web application, wherein web applications are responsive versions of websites that can work on any mobile device or operating system because they are delivered using a mobile browser. A mobile application may be a hybrid application, wherein hybrid applications are combinations of both native and web apps, but wrapped within a native app, giving it the ability to have its own icon or be downloaded from an app store. A mobile application may be one or both of an executable file, or one or more files needing compiling for use on a desktop or server computing device.

“Nodes” and “Vertices” are used herein interchangeably to mean a unit of a data structure comprising a value, condition, or other information. Nodes and vertices may be arranged in lists, trees, graphs, and other forms of data structures. In graphs, nodes and vertices may be connected to an arbitrary number of edges, which represent relationships between the nodes or vertices. As the context requires, the term “node” may also refer to a node of a neural network (also referred to as a neuron) which is analogous to a graph node in that it is a point of information connected to other points of information through edges.

“Normalized pointwise mutual information” (NPMI) as used herein is the measure of how much the actual probability of a particular co-occurrence of events (word-pairs) differs from its expected probability on the basis of the probabilities of the individual events and the assumption of independence. The calculated NPMI value is bounded between the values of negative one and one (−1, 1), inclusive. A value of negative one indicates the word-pair occur separately, but never occur together. A value of zero indicates independence of the word-pair in which co-occurrences happen at random. A value of one indicates complete co-occurrence, or that the word-pair only exist together.

“Outcome” as used herein is a measure within a clinical trial which is used to assess the effect, both positive and negative, of an intervention or treatment. In clinical trials such measures of direct importance of for an individual may include, but are not limited to, survival, quality of life, morbidity, suffering, functional impairment, and changes in symptoms.

“Pocket” or “Protein binding pocket” as used herein means a cavity (i.e., receptor, binding site) on the surface or in the interior of a protein that possesses suitable properties for binding a ligand. The set of amino acid residues around a binding pocket determines its physicochemical characteristics and, together with its shape and location in a protein, defines its functionality.

“Pose” as used herein means a molecule within a protein binding site arranged in a certain conformation.

“Proteins” as used herein means large biomolecules, or macromolecules, consisting of one or more long chains of amino acid residues. Proteins perform a vast array of functions within organisms, including catalyzing metabolic reactions, DNA replication, responding to stimuli, providing structure to cells and organisms, and transporting molecules from one location to another. Proteins differ from one another primarily in their sequence of amino acids, which is dictated by the nucleotide sequence of their genes, and which usually results in protein folding into a specific 3D structure that determines its activity.

“SAE” and “AE” as used herein means serious adverse effects (SAE) and adverse effects (AE), as relating to biological biomarkers in clinical trial patients.

“SMILES” as used herein means any version of the “simplified molecular-input line-entry system,” which is form of chemical notation for describing the structure of molecules using short text (e.g., ASCII) strings.

### Conceptual Architecture

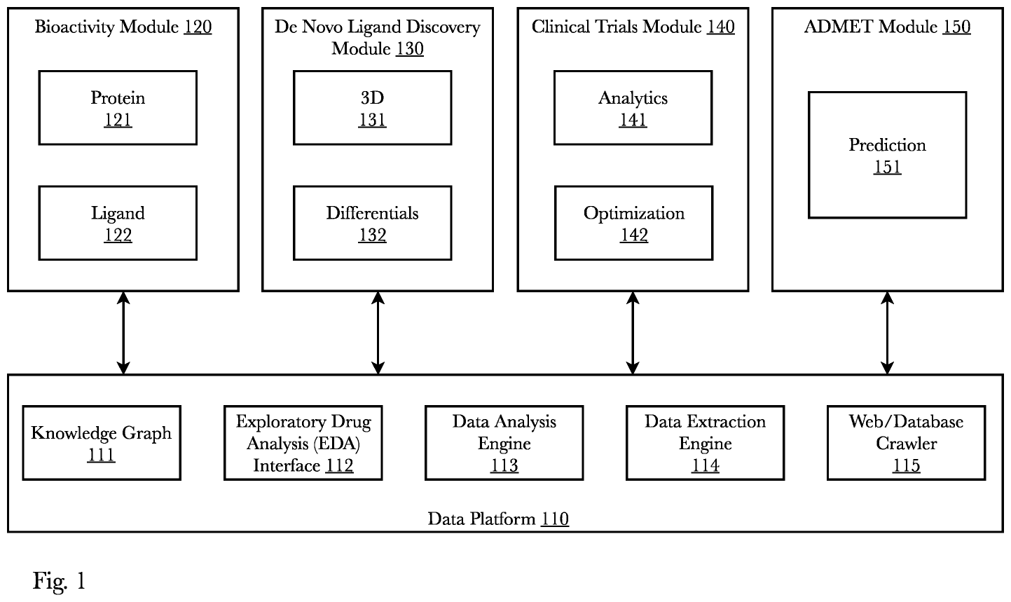

FIG. 1 is a block diagram illustrating an exemplary overall system architecture for a pharmaceutical research system. The exemplary architecture comprises a data platform 110 which provides the core functionality of the system, plus one or more modules that utilize the data platform 110 to provide functionality in specific areas of research, in this case a bioactivity module 120, a de novo ligand discovery module 130, a clinical trials module 140, and an absorption, distribution, metabolism, excretion, and toxicity (ADMET) module 150.

The data platform 110 in this embodiment comprises a knowledge graph 111, an exploratory drug analysis (EDA) interface 112, a data analysis engine 113, a data extraction engine 114, and web crawler/database crawler 115. The crawler 115 searches for and retrieves medical information such as published medical literature, clinical trials, dissertations, conference papers, and databases of known pharmaceuticals and their effects. The crawler 115 feeds the medical information to a data extraction engine 114, which uses natural language processing techniques to extract and classify information contained in the medical literature such as indications of which molecules interact with which proteins and what physiological effects have been observed. Using the data extracted by the data extraction engine 114, a knowledge graph 111 is constructed comprising vertices (also called nodes) representing pieces of knowledge gleaned from the data and edges representing relationships between those pieces of knowledge. As a very brief example, it may be that one journal article suggests that a particular molecule is useful in treating a given disease, and another journal article suggests that a different molecule is useful for treating the same disease. The two molecules and the disease may be represented as vertices in the graph, and the relationships among them may be represented as edges between the vertices. The EDA interface 112 is a user interface through which pharmaceutical research may be performed by making queries and receiving responses. The queries are sent to a data analysis engine 113 which uses the knowledge graph 111 to determine a response, which is then provided to the user through the EDA interface 112. In some embodiments, the data analysis engine 113 comprises one or more graph-based neural networks (graph neural networks, or GNNs) to process the information contained in the knowledge graph 111 to determine a response to the user's query. As an example, the user may submit a query for identification of molecules likely to have similar bioactivity to a molecule with known bioactivity. The data analysis engine 113 may process the knowledge graph 111 through a GNN to identify such molecules based on the information and relationships in the knowledge graph 111.

The bioactivity module 120 utilizes the data platform 110 to analyze and predict the bioactivity of molecules based on protein 121 and ligand 122 similarities and known or suspected protein 121 and ligand 122 compatibilities. The module utilizes the knowledge graph 111 and data analysis engine 113 capabilities of the data platform 110, and in one embodiment is configured to predict the bioactivity of a molecule based on and their known or suspected compatibilities with certain combinations of proteins 121 and ligands 122. Thus, using the bioactivity module 120, users can research molecules by entering queries through the EDA interface 112, and obtaining using predictions of bioactivity based on known or suspected bioactivity of similar molecules and their compatibilities with certain protein 121 and ligand 122 combinations.

The de novo ligand discovery module 130 utilizes the data platform 110 to identify ligands and their properties through data enrichment and interpolation/perturbation. The module utilizes the knowledge graph 111 and data analysis engine 113 capabilities of the data platform 110, and in one embodiment is configured to identify ligands with certain properties based on three dimensional (3D) models 131 of known ligands and differentials of atom positions 132 in the latent space of the models after encoding by a 3D convolutional neural network (3D CNN), which is part of the data analysis engine 113. In one embodiment, the 3D model comprises a voxel image (volumetric, three dimensional pixel image) of the ligand. In cases where enrichment data is available, ligands may be identified by enriching the SMILES string for a ligand with information about possible atom configurations of the ligand and converting the enriched information into a plurality of 3D models of the atom. In cases where insufficient enrichment information is available, one possible configuration of the atoms of the ligand may be selected, and other configurations may be generated by interpolation or perturbation of the original configuration in the latent space after processing the 3D model through the CNN. In either case, the 3D models of the ligands are processed through a CNN, and a gradient descent is applied to changes in atom configuration in the latent space to identify new ligands with properties similar to the modeled ligands. Thus, using the de novo ligand discovery module 130, users can identify new ligands with properties similar to those of modeled ligands by entering queries through the EDA interface 112.

The clinical trials module 140 utilizes the data platform 110 to analyze 141 and optimize 142 the knowledge contained in or derived from clinical trials. The module utilizes the knowledge graph 111 and data analysis engine 113 capabilities of the data platform 110, and in one embodiment is configured to return clinical trials similar to a specified clinical trial in one or more aspects (e.g., proteins and ligands studied, methodology, results, etc.) based on semantic clustering within the knowledge graph 111. Thus, using the clinical trials module 140, users can research a large database of clinical trials based on aspects of interest by entering queries through the EDA interface 112.

The ADMET module 150 utilizes the data platform 110 to predict 151 absorption, distribution, metabolism, excretion, and toxicity characteristics of ligands based on ADMET databases. The module utilizes the knowledge graph 111 and data analysis engine 113 capabilities of the data platform 110, and in one embodiment is configured to return ligands with characteristics similar to, or dissimilar to, a specified ligand in one or more respects (e.g., a ligand with similar absorption and metabolism characteristics, but dissimilar toxicity characteristics) based on semantic clustering within the knowledge graph 111. Thus, using the ADMET module 150, users can research a large ADMET database based on aspects of interest by entering queries through the EDA interface 112.

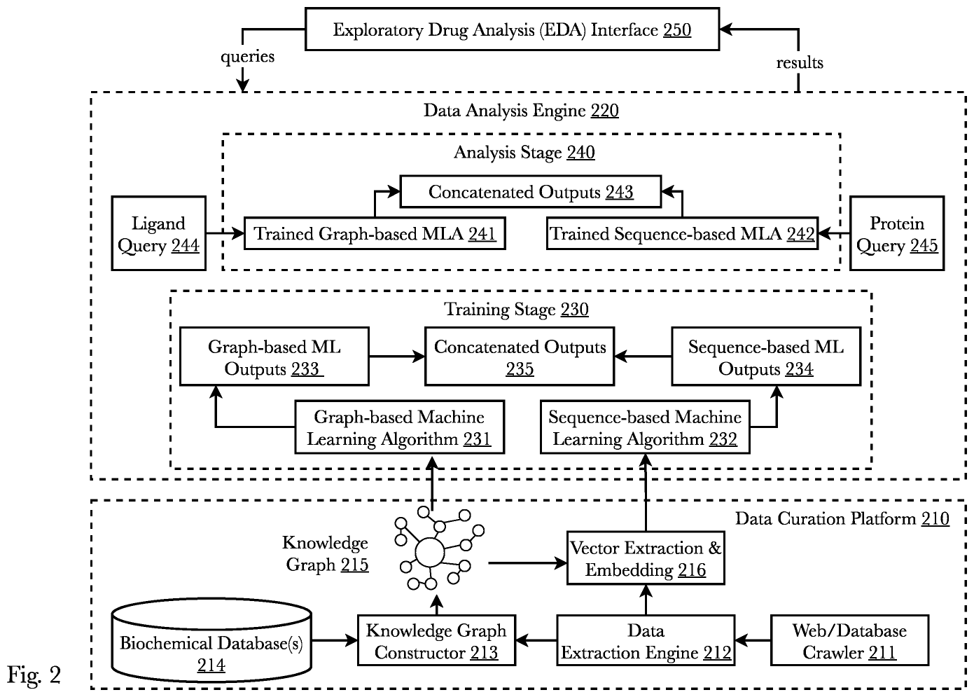

FIG. 2 is a block diagram illustrating an exemplary system architecture for an embodiment of a pharmaceutical research system utilizing combined graph-based and sequence-based prediction of molecule bioactivity. In this embodiment, the system comprises a data curation platform 210, a data analysis engine 220 comprising a training stage 230 and an analysis stage 240, and an exploratory drug analysis interface 250. The knowledge graph 215 does not refer to a graph representation of the inputs to the model, but to a relational structure of the data in the database itself. The knowledge graph 215 itself is not used as input.

In the data curation platform 210, a web crawler/database crawler 211 is configured to search for and download medical information materials including, but not limited to, archives of published medical literature such as MEDLINE and PubMed, archives of clinical trial databases such as the U.S. National Library of Medicine's ClinicalTrials.gov database and the World Health Organization International Clinical Trials Registry Platform (ICTRP), archives of published dissertations and theses such as the Networked Digital Library of These and Dissertations (NDLTD), archives of grey literature such as the Grey Literature Report, and news reports, conference papers, and individual journals. As the medical information is downloaded, it is fed to a data extraction engine 212 which may perform a series of operations to extract data from the medical information materials. For example, the data extraction engine 212 may first determine a format of each of the materials received (e.g., text, PDFs, images), and perform conversions of materials not in a machine-readable or extractable format (e.g., performing optical character recognition (OCR) on PDFs and images to extract any text contained therein). Once the text has been extracted from the materials, natural language processing (NLP) techniques may be used to extract useful information from the materials for use in analysis by machine learning algorithms. For example, semantic analysis may be performed on the text to determine a context of each piece of medical information material such as the field of research, the particular pharmaceuticals studied, results of the study, etc. Of particular importance is recognition of standardized biochemistry naming conventions including, but not limited to, stock nomenclature, International Union of Pure and Applied Chemistry (IUPAC) conventions, and simplified molecular-input line-entry system (SMILES) and FASTA text-based molecule representations. The data extraction engine 212 feeds the extracted data to a knowledge graph constructor 213, which constructs a knowledge graph 215 based on the information in the data, representing informational entities (e.g., proteins, molecules, diseases, study results, people) as vertices of a graph and relationships between the entities as edges of the graph. Biochemical databases 214 or similar sources of information may be used to supplement the graph with known properties of proteins, molecules, physiological effects, etc. Separately from the knowledge graph 215, vector representations of proteins, molecules, interactions, and other information may be represented as vectors 216, which may either be extracted from the knowledge graph 215 or may be created directly from data received from the data extraction engine 212. The link between the knowledge graph 215 and the data analysis engine 220 is merely an exemplary abstraction. The knowledge graph 215 does not feed into the models directly but rather the data contained in a knowledge graph structured database is used to train the models. The same exemplary abstraction applies between the vector extraction and embedding 216 and the data analysis engine 220.

The data analysis engine 220 utilizes the information gathered, organized, and stored in the data curation platform 210 to train machine learning algorithms at a training stage 230 and conduct analyses in response to queries and return results based on the analyses at an analysis stage 240. The training stage 230 and analysis stage 240 are identical, whereas the analysis stage 240 has already completed training. In this embodiment, the data analysis engine 220 comprises a dual analysis system which combines the outputs of a trained graph-based machine learning algorithm 241 with the outputs of a trained sequence-based machine learning algorithm 242. The trained graph-based machine learning algorithm 241 may be any type of algorithm configured to analyze graph-based data, such as graph traversal algorithms, clustering algorithms, or graph neural networks.

At the training stage 230, information from the knowledge graph 215 is extracted to provide training data in the form of graph-based representations of molecules and the known or suspected bioactivity of those molecules with certain proteins. The graph-based representations, or 3D representations in the 3D case, of the molecules and proteins and their associated bioactivities are used as training input data to a graph-based machine learning algorithm 231, resulting in a graph-based machine learning output 233 comprising vector representations of the characteristics of molecules and their bioactivities with certain proteins. Simultaneously, a sequence-based machine learning algorithm is likewise trained, but using information extracted 216 from the knowledge graph 215 in the form of vector representations of protein segments and the known or suspected bioactivity of those protein segments with certain molecules. The vector representations of the protein segments and their associated bioactivities are used to train the concatenated outputs 235, as well as the machine learning algorithms 231, 232, 233, 234. In this embodiment, the graph-based machine learning outputs 233 and the sequence-based machine learning outputs 234 are concatenated to produce a concatenated output 235, which serves to strengthen the learning information from each of the separate machine learning algorithms. In this and other embodiments, the concatenated output may be used to re-train both machine learning algorithms 233, 234 to further refine the predictive abilities of the algorithms.

At the analysis stage, a query in the form of a target ligand 244 and a target protein 245 are entered using an exploratory drug analysis (EDA) interface 250. The target ligand 244 is processed through the trained graph-based machine learning algorithm 241 which, based on its training, produces an output comprising a vector representation of the likelihood of interaction of the target ligand 244 with certain proteins and the likelihood of the bioactivity resulting from the interactions. Similarly, the target protein 245 is processed through the trained sequence-based machine learning algorithm 242 which, based on its training, produces an output comprising a vector representation of the likelihood of interaction of the target protein 245 with certain ligands and the likelihood of the bioactivity resulting from the interactions. The results may be concatenated 243 to strengthen the likelihood information from each of the separate trained machine learning algorithms 241, 242.

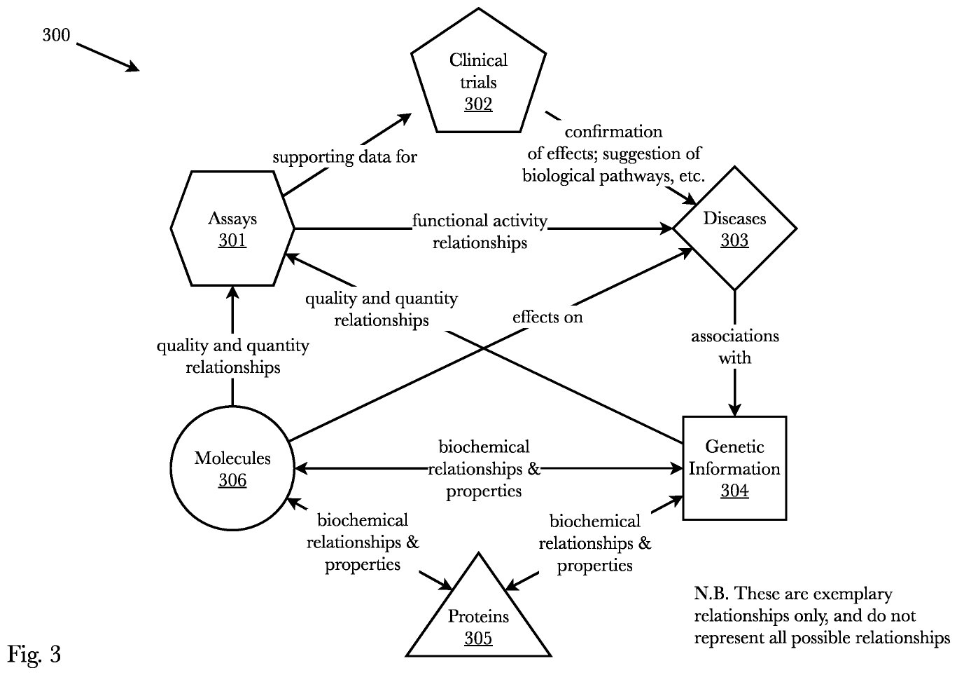

FIG. 3 is a relational diagram 300 illustrating several types of information that may be included in a knowledge graph for a pharmaceutical research system and exemplary relations between those types of information. In this example, six types of information are shown with indications of certain relevant relationships and interactions that may be represented in a knowledge graph containing these types of information. The six types of information in this example are chosen to be of particular relevance to pharmaceutical research, and in particular to the analysis of, and prediction of, biochemical properties of proteins and ligands as they relate to disease. Proteins 305 and molecules (ligands) 306 are the primary types of information, as their biochemical relationships and properties determine effects on diseases 303. Genetic information 304 will have an influence on the production of specific proteins 305 and the association with certain diseases 303. Assays 301 will provide information about the quality and quantity relationships of proteins 350 and molecules 306, which provides supporting data for clinical trials 302 and for functional activity relationships with certain diseases 303. Clinical trials 302 provide confirmation of physiological effects and suggestion of biological pathways related to diseases. While this simplified diagram does not purport to show all types of data that may be included or all relationships that may be relevant, it does show certain important types of data and major relevancies that may be included in a knowledge graph to be used for a pharmaceutical research system.

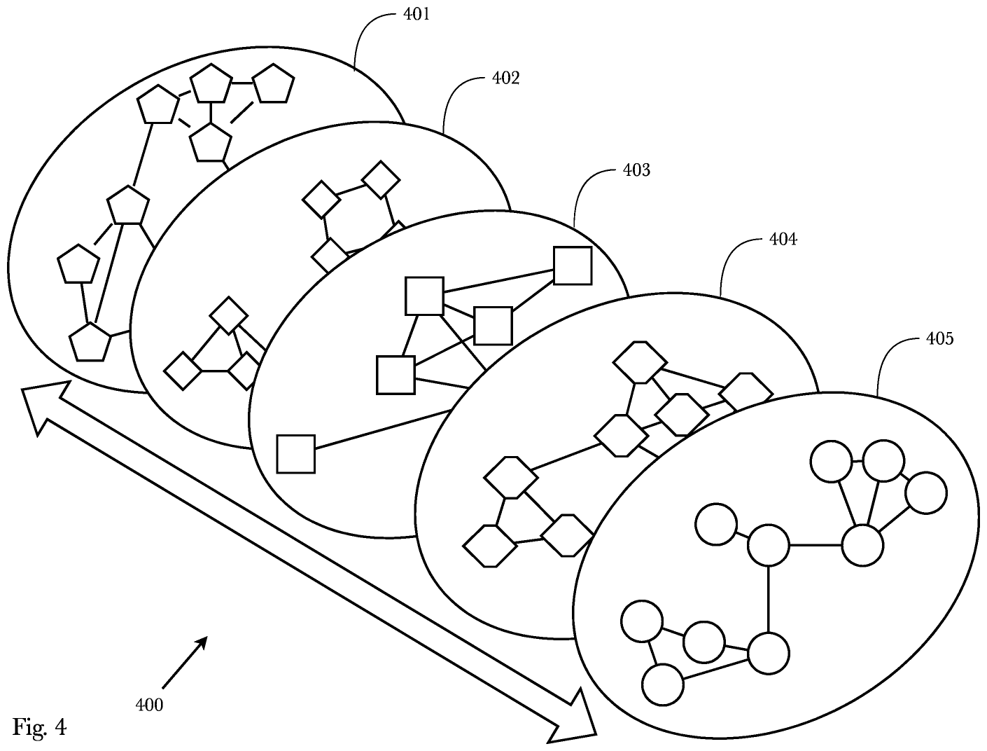

FIG. 4 is a diagram illustrating the conceptual layering 400 of different types of information in a knowledge graph. While knowledge graphs are not necessarily constructed in layers, each type of information included in a knowledge graph may be conceived as a layer of information in the knowledge graph and each layer may be analyzed to determine clustering and other relationships within the layer. For example, proceeding with the types of information shown in FIG. 3, the knowledge graph can be conceived of as having layers for clinical trials 401, diseases 402, genetic information 403, assays 404, molecules 405, etc. Relationships such as clustering can be seen at each layer, and can be analyzed separately, if necessary. However, in a knowledge graph, connections between the information at each layer are made and relationships between the information at each layer can be analyzed.

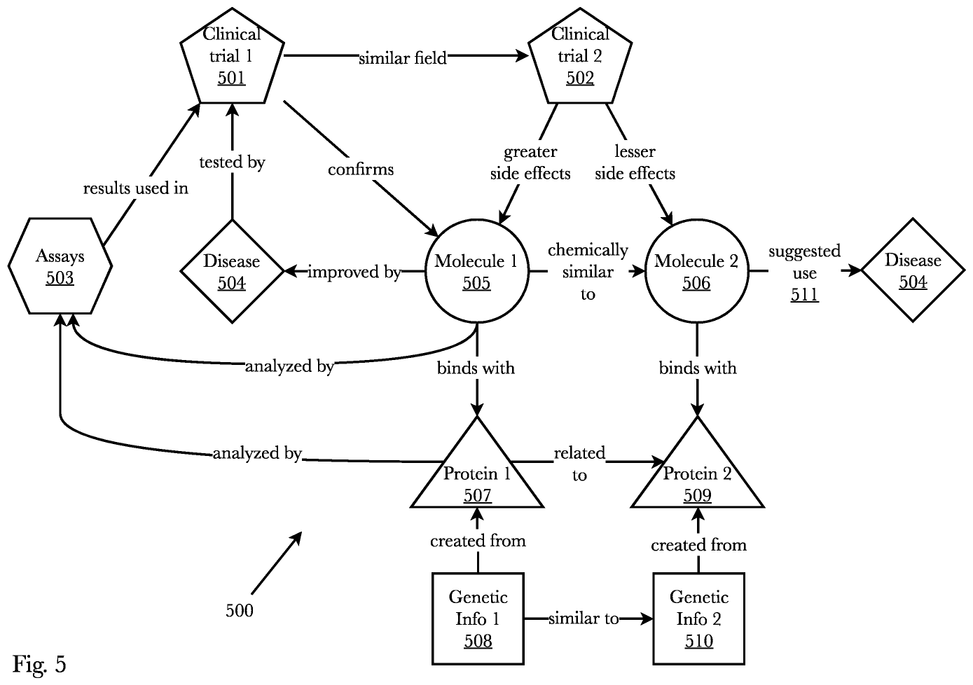

FIG. 5 is a relational diagram illustrating the use of a knowledge graph to predict usefulness of a molecule in treating a disease 500. In this example, a first molecule 505 is known to bind with a first protein 507 which is produced from a first set of genetic information 508. A clinical trial 501 confirmed that the first molecule 505 is effective in treating a disease 504. The clinical trial 501 used information from assays 503 that were performed on the first molecule 505 and the first protein 507. A query has been submitted to the system to identify a second molecule 506 that may also be effective in treating 511 the same disease 504, but with fewer side effects. Using a knowledge graph containing the types of information shown in FIG. 3, and a graph-based machine learning algorithm, the system identifies a second molecule 506 that binds with a second protein 509 which is produced from a second set of genetic information 510. The system determines a number of similarities and relationships between the first molecule 505 and the second molecule 506, including that the first molecule 505 is chemically similar to the second molecule 506, the protein 507 with which the first molecule 505 binds is related to the second protein 509 with which the second molecule 506 binds, and the genetic information (DNA strands) 508 that produces the first protein 507 are similar to the genetic information 510 that produces the second protein 509. Thus, the system determines that the second molecule 506 is likely to have a similar effect on the disease 504 as the first molecule 505. Further, the system identifies a second clinical trial 502 that suggests that the second molecule 506 has lesser side effects than the first molecule 505. As the second molecule 506 meets the query criteria, it is returned as a response to the query.

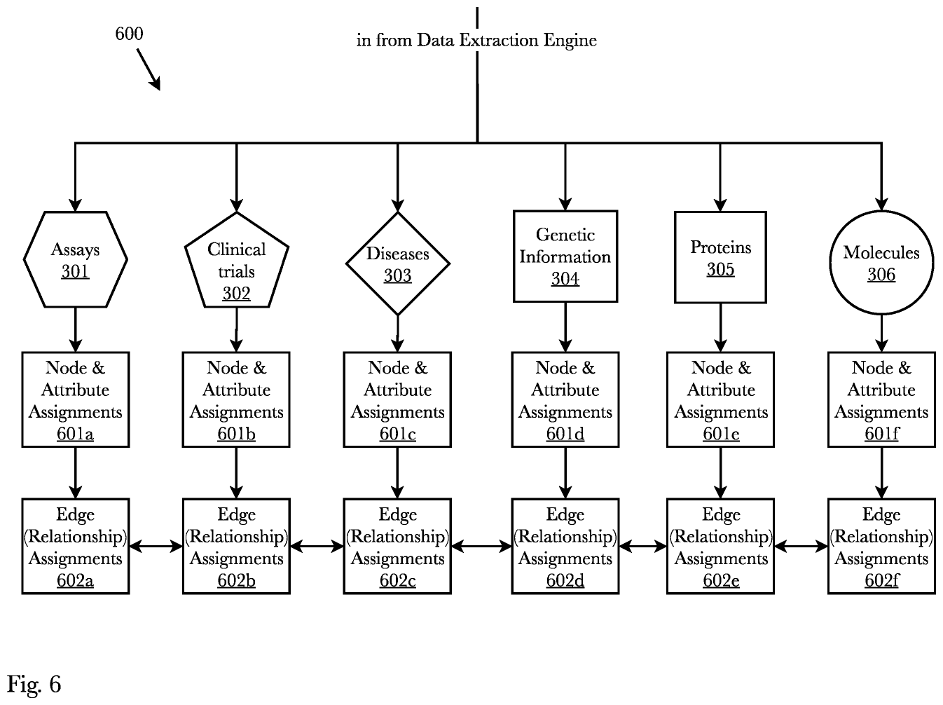

FIG. 6 is a diagram illustrating an exemplary process 600 for combining various types of information into a knowledge graph suitable for a pharmaceutical research system. As data is received from a data extraction engine in each of several categories of data (in this example, six categories: assays 301, clinical trials 302, diseases 303, genetic information 304, proteins 305, and molecules 306) nodes are assigned to each entity identified in each category and attributes of the entity are assigned to the node 601a-f. Attributes of the nodes/entity are information describing the characteristics of the nodes/entity. For example, in some embodiments, attributes of nodes related to molecules are in the form of an adjacency matrix which represents the molecule as relationships between the atoms of the molecule. After nodes have been assigned to all identified entities 601a-f, the relationships between entities are assigned, both within the category of knowledge and between all other categories of knowledge 602a-f. As a simple example of the process, assume that a certain molecule 306 is identified during data extraction. A node is created for the molecule and attributes are assigned to the molecule/node in the form of an adjacency matrix representing the molecule as a series of relationships between the atoms of the molecule. Through a series of assays 301 and clinical studies 302, it is known that the molecule binds with a particular protein 305, and is effective in treating a certain disease 303, to which individuals with certain genetic information 304 are susceptible. Nodes are assigned to each of the assays 301, clinical trials 302, diseases 303, proteins 305, and genetic information 304 identified as being associated with the molecule, and edges are established between the nodes reflecting the relevant relationships such as: the molecule binds with the protein, the genetic information is associated with the disease, the clinical trials indicate that the disease is treatable by the molecule, and so on.

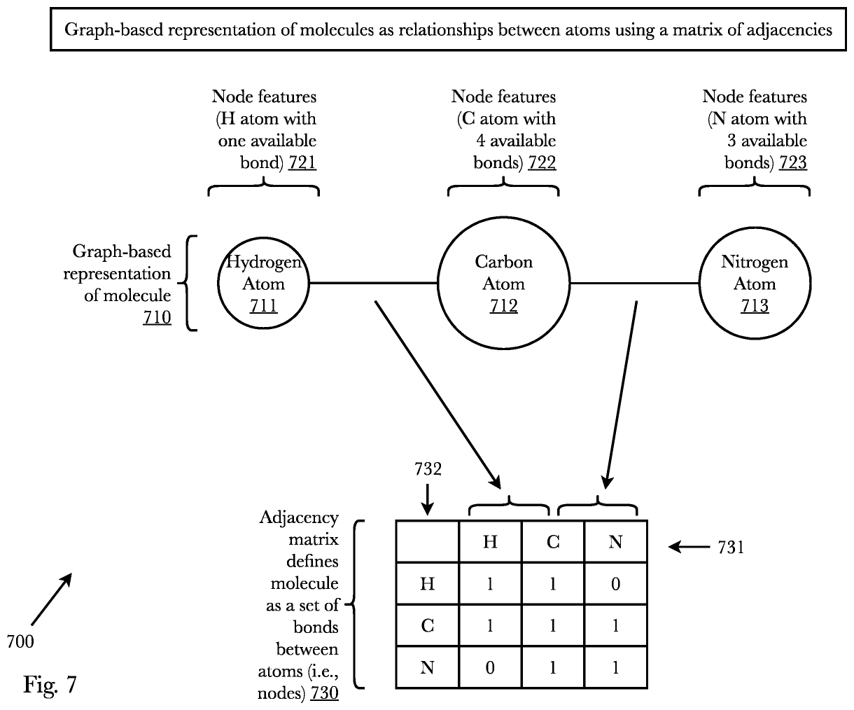

FIG. 7 is a diagram illustrating an exemplary graph-based representation of molecules as simple relationships between atoms using a matrix of adjacencies 700, wherein atoms are represented as nodes and bonds between the atoms are represented as edges. Representation of molecules as a graph is useful because it provides a molecular structure which can be processed by graph-based machine learning algorithms like GNNs. Further, the graph-based representation of a molecule can be stated in terms of two matrices, one for the node features (e.g., type of atom and its available bonds) and one for the edges (i.e., the bonds between the atoms). The combination of the nodes (atoms) and edges (bonds) represents the molecule. Each molecule represented in the matrix comprises a dimensionality and features that describe the type of bond between the atoms. According to one embodiment, all bonds within the graph hold the same value, e.g., 1. However, in other embodiments, bonds may be differentiated such as hydrogen bonds having a value of 3, or by having the bond feature dimension exist in each cell.

In this example, a simple hydrogen cyanide molecule is shown as a graph-based representation 710. A hydrogen cyanide molecule consists of three atoms, a hydrogen atom 711, a carbon atom 712, and a nitrogen atom 713. Its standard chemical formula is HCN. Each atom in the molecule is shown as a node of a graph. The hydrogen atom 711 is represented as a node with node features 721 comprising the atom type (hydrogen) and the number of bonds available (one). The carbon atom 712 is represented as a node with node features 722 comprising the atom type (carbon) and the number of bonds available (four). The nitrogen atom 713 is represented as a node with node features 723 comprising the atom type (nitrogen) and the number of bonds available (three). The node features 721, 722, 723 may each be stated in the form of a matrix.

The relationships between the atoms in the molecule are defined by the adjacency matrix 730. The top row of the adjacency matrix 731 shows all of the atoms in the molecule, and the left column of the matrix 732 shows a list of all possible atoms that can be represented by the matrix for a given set of molecules. In this example, the top row 731 and left column 732 contain the same list of atoms, but in cases where multiple molecules are being represented in the system, the left column may contain other atoms not contained in the particular molecule being represented. The matrix shows, for example, that the hydrogen atom 711 is connected to the carbon atom 712 (a “1” at the intersection of the rows and columns for H and C) and that the carbon atom 712 is connected to the nitrogen atom 713 (a “1” at the intersection of the rows and columns for C and N). In this example, each atom is also self-referenced (a “1” at the intersection of the rows and columns for H and H, C and C, and N and N), but in some embodiments, the self-referencing may be eliminated. In some embodiments, the rows and columns may be transposed (not relevant where the matrix is symmetrical, but relevant where it is not).

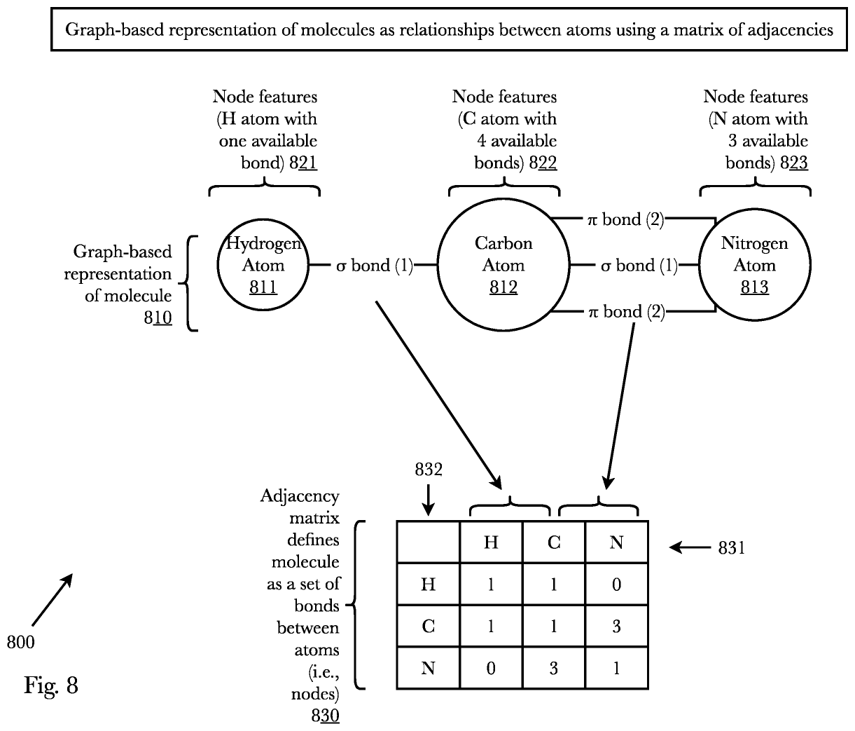

FIG. 8 is a diagram illustrating an exemplary graph-based representation of molecules as relationships between atoms using a matrix of adjacencies 800, wherein atoms are represented as nodes and bonds between the atoms are represented as edges, and wherein the type and number of bonds are distinguished. Representation of molecules as a graph is useful because it provides a molecular structure which can be processed by graph-based machine learning algorithms like GNNs. Further, the graph-based representation of a molecule can be stated in terms of two matrices, one for the node features (e.g., type of atom and its available bonds) and one for the edges (i.e., the bonds between the atoms). The combination of the nodes (atoms) and edges (bonds) represents the molecule.

In this example, a simple hydrogen cyanide molecule is shown as a graph-based representation 810. A hydrogen cyanide molecule consists of three atoms, a hydrogen atom 811, a carbon atom 812, and a nitrogen atom 813. Its standard chemical formula is HCN. Each atom in the molecule is shown as a node of a graph. The hydrogen atom 811 is represented as a node with node features 821 comprising the atom type (hydrogen) and the number of bonds available (one). The carbon atom 812 is represented as a node with node features 822 comprising the atom type (carbon) and the number of bonds available (four). The nitrogen atom 813 is represented as a node with node features 823 comprising the atom type (nitrogen) and the number of bonds available (three). The node features 821, 822, 823 may each be stated in the form of a matrix.

The relationships between the atoms in the molecule are defined by the adjacency matrix 830. The top row of the adjacency matrix 831 shows all of the atoms in the molecule, and the left column of the matrix 832 shows a list of all possible atoms that can be represented by the matrix for a given set of molecules. In this example, the top row 831 and left column 832 contain the same list of atoms, but in cases where multiple molecules are being represented in the system, the left column may contain other atoms not contained in the particular molecule being represented. The matrix shows, for example, that the hydrogen atom 811 is connected to the carbon atom 812 (a “1” at the intersection of the rows and columns for H and C) and that the carbon atom 812 is connected to the nitrogen atom 813 (a “3” at the intersection of the rows and columns for C and N). In this example, the number of bonds between atoms is represented by the digit in the cell of the matrix. For example, a 1 represents a single bond, whereas a 3 represents a triple bond. In this example, each atom is also self-referenced (a “1” at the intersection of the rows and columns for H and H, C and C, and N and N), but in some embodiments, the self-referencing may be eliminated. In some embodiments, the rows and columns may be transposed (not relevant where the matrix is symmetrical, but relevant where it is not).

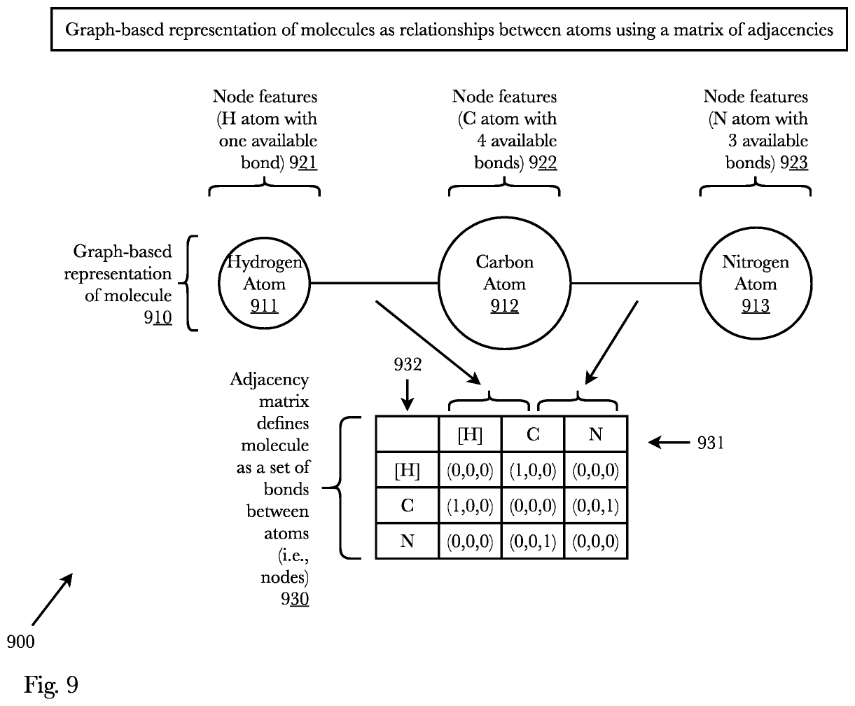

FIG. 9 is a diagram illustrating an exemplary graph-based representation of molecules as relationships between atoms using a matrix of adjacencies 900, wherein atoms are represented as nodes and bonds between the atoms are represented as edges, and wherein the matrix of adjacencies uses a SMILES string encoding of the molecule and one-hot vector representations of the type of bonds between atoms in the molecule. Representation of molecules as a graph is useful because it provides a molecular structure which can be processed by graph-based machine learning algorithms like GNNs. Further, the graph-based representation of a molecule can be stated in terms of two matrices, one for the node features (e.g., type of atom and its available bonds) and one for the edges (i.e., the bonds between the atoms). The combination of the nodes (atoms) and edges (bonds) represents the molecule.

In this example, a simple hydrogen cyanide molecule is shown as a graph-based representation 910. A hydrogen cyanide molecule consists of three atoms, a hydrogen atom 911, a carbon atom 912, and a nitrogen atom 913. Its SMILES representation text string is [H]C #N, with the brackets around the H indicating an element other than an organic element, and the # representing a triple bond between the C and N. Each atom in the molecule is shown as a node of a graph. The hydrogen atom 911 is represented as a node with node features 921 comprising the atom type (hydrogen) and the number of bonds available (one). The carbon atom 912 is represented as a node with node features 922 comprising the atom type (carbon) and the number of bonds available (four). The nitrogen atom 913 is represented as a node with node features 923 comprising the atom type (nitrogen) and the number of bonds available (three). The node features 921, 922, 923 may each be stated in the form of a matrix 930.

In this example, the top row 931 and left column 932 contain the same list of atoms, but in cases where multiple molecules are being represented in the system, the left column may contain other atoms not contained in the particular molecule being represented. The matrix shows, for example, that the hydrogen atom 811 is connected to the carbon atom 812 with a single bond (the one-hot vector “(1,0,0)” at the intersection of the rows and columns for H and C) and that the carbon atom 812 is connected to the nitrogen atom 813 with a triple bond (the one-hot vector “(0,0,1)” at the intersection of the rows and columns for C and N). In this example, the number of bonds between atoms is represented by a one-hot vector in the cell of the matrix. For example, a 1 in the first dimension of the vector (1,0,0) represents a single bond, whereas a 1 in the third dimension of the vector (0,0,1) represents a triple bond. In this example, self-referencing of atoms is eliminated, but self-referencing may be implemented in other embodiments, or may be handled by assigning self-referencing at the attention assignment stage. In some embodiments, the rows and columns may be transposed (not relevant where the matrix is symmetrical, but relevant where it is not).

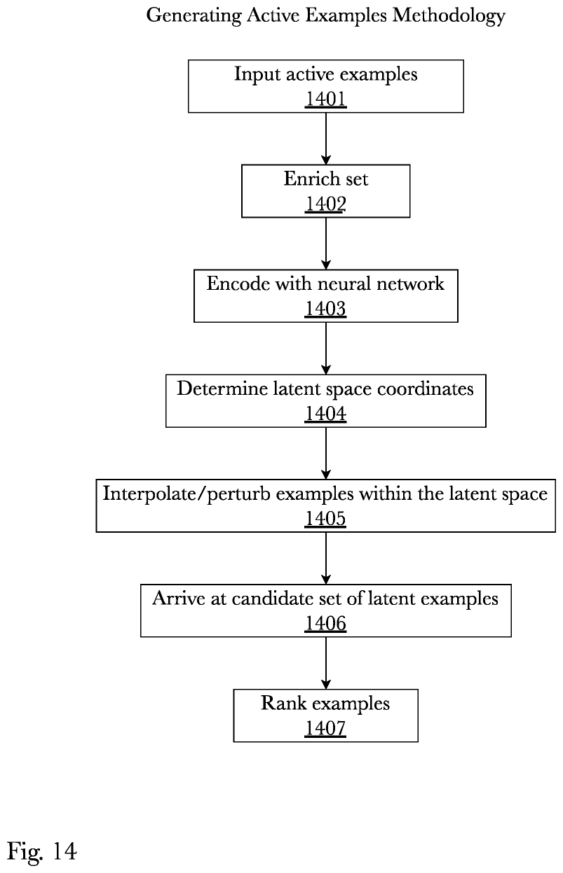

FIG. 14 is a flow diagram illustrating an exemplary method for active example generation. According to a general methodology description, generating active examples (i.e., chemically valid ligand-receptor pairs) is performed by the first step of gathering known active examples from databases, web-crawlers, and other sources previously described in past figures 1401. Active examples may then be enriched to fill in missing data, supplement, append or otherwise enhance the training data 1402. A specific example of enrichment may be finding similar compounds with the same properties as a target molecule or that responds to known ligands in the same fashion. With the enhanced training data (i.e., enriched active examples) gathered, it is fed into a neural network (NN) 1403. A consideration must be noted that many machine learning algorithms exist, and that this method may work with many NN models or other machine learning algorithms and is not limited to the ones disclosed herein.

The neural networks build a model from the training data. In the case of using an autoencoder (or a variational autoencoder), the encoder portion of the neural network reduces the dimensionality of the input molecules, learning a model from which the decoder portion recreates the input molecule. The significance of outputting the same molecule as the input is that the decoder may then be used as a generative function for new molecules. One aspect of a generative decoder module is that the learned model (i.e., protein-ligand atom-features according to one embodiment) lies in a latent space 1404. Sampled areas of the latent space are then interpolated and perturbed 1405 to alter the model such that new and unique latent examples 1406 may be discovered. Other ways to navigate the latent space exist, Gaussian randomization as one example, that may be used in other embodiments of the invention. Furthermore, libraries, other trained models, and processes exist that may assist in the validation of chemically viable latent examples within the whole of the latent space; processing the candidate set of latent examples through a bioactivity model, as one example 1407.

Regarding retrosynthesis for de novo drug design, two approaches are described below. A first approach begins with preprocessing all the SMILES representations for reactants and products to convert to canonical form (SMILES to Mol & Mol to SMILES through a cheminformatics toolkit), remove duplicates & clean the data, augmenting SMILE equivalents via enumeration. Then, transformer models are used with multiple attention heads and a k-beam search is set up. Further, the models are conformed by optimizing on producing long-term reactants, ensuring the models are robust to different representations of a molecule, providing intrinsic recursion (using performers), and including further reagents such as catalysts and solvents.

A second approach begins with augmenting the transformer model with a hyper-graph approach. Starting with an initial node of the graph as the query molecule and recursively: the molecule with highest upper-bound confidence (UCB) score is selected (specifically, the UCB is adapted to trees generation UCT), the node is expanded (if this node is not terminal), and expansions from that node are simulated to recover a reward. Rewards are backpropagated along the deque of selected nodes, and the process is repeated until convergence. Here UCB is used as a form of balancing exploration-exploitation, where X is the reward, n is the number of times the parent node has been visited, j denotes the child node index, and Cₚ (>0) is an exploration constant. In one embodiment, the model may be constrained to a rewarding a node when its children are accessible, wherein other embodiments may use rewards such as molecular synthesis score, LogP, synthesis cost, or others known in the art.

UCT=Xⱼ+2Cₚ\sqrt{2 ln n/nⱼ}

According to one aspect of the second approach, transformer models are optimized so that they produce a molecule that can be formed with another molecule. However, these models should be optimized with the aim of producing reactants which are going to recursively deconstruct into accessible molecules. Hence, adding reinforcement learning finetuning to force the transformer model to not only produce reactants which are plausible but to produce reactants which lead to favorable retrosynthetic routes.

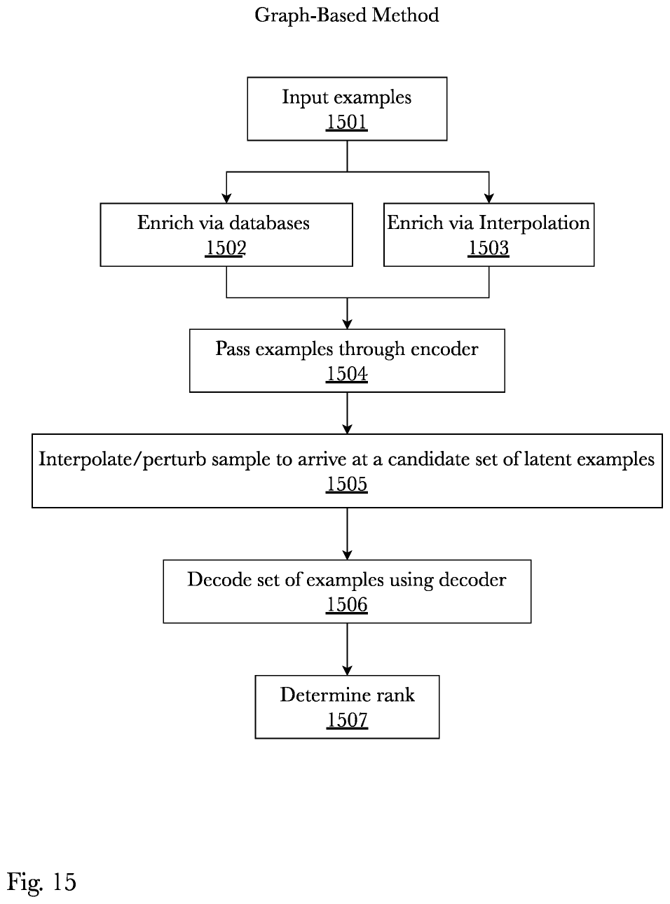

FIG. 15 is a flow diagram illustrating an exemplary method for active example generation using a graph-based approach. According to a first preferred embodiment of active example generation, where a graph-based method is used, active molecules are input (via a WebApp according to one aspect) as SMILES representations 1501. This involves training an autoencoder to obtain a fixed-dimensional representation of SMILES and may further be reused for the bioactivity model. Additionally, standard SMILES encoding fails to capture all pertinent information relating to the atoms (e.g., bond length). Consequently, enumeration may be used to improve the standard SMILES model where enumeration is an equivalent to data augmentation via rotation, therefore by having different SMILES representations of the same molecule from different orientations the missing information is captured. Other enumeration methods may be used where data is necessary but missing. The enumerated SMILES encoding used may comprise one-hot encodings of atom type, atom degree, valence, hybridization, and chirality as well as formal charge and number of radical electrons. Bond types (single, double, triple, and aromatic), bond length, and bond conjugation with ring and stereo features are also captured.

Enrichment of the input data may be performed by searching through data sets for similar compounds through specific tags (e.g., anti-viral) 1502. Additionally, the enrichment process may be used if the training data lacks any descriptive parameters, whereby databases, web-crawlers, and such may fill in the missing parameters 1502. Enrichment may also occur where data is sparse by interpolating between known molecules 1503. This enriched training data is then captured in node and edge feature matrices. Some embodiments may use matrices comprising a node feature matrix, N, of shape (No_Atoms, No_Features_Atom) and edge feature (adjacency) tensor, A, of shape (No_Atoms, No_Atoms, No_Features_Bond). A reminder to the reader that a tensor's rank is its matrix dimensionality.

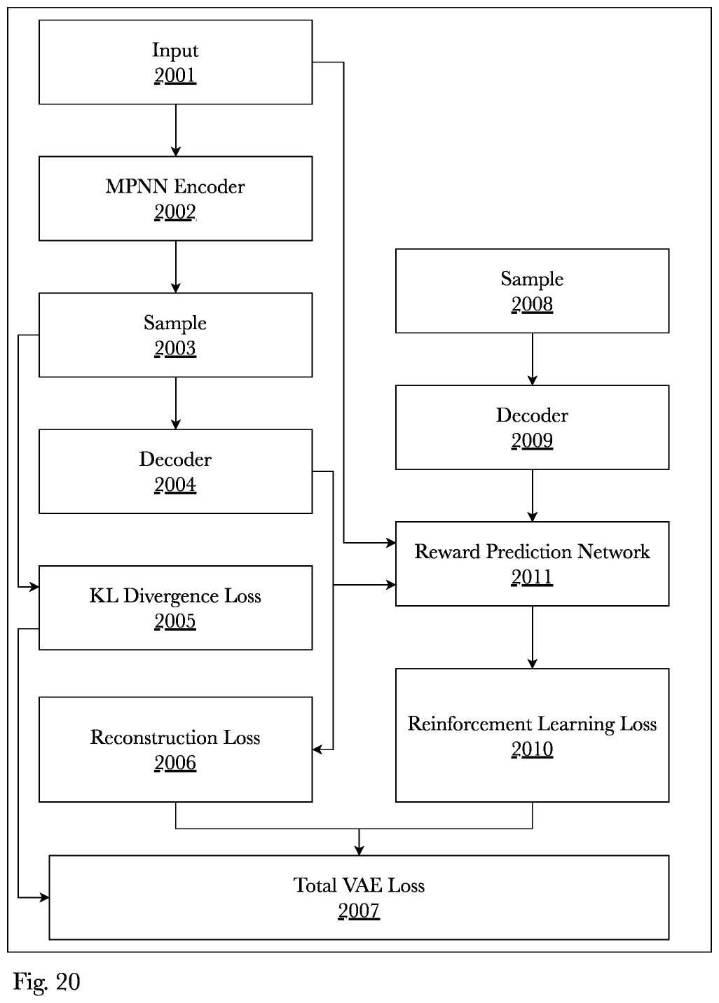

The next step is to pass examples through a variational autoencoder (VAE) together with a reinforcement learning component to build the full model 1504 (See FIG. 20). The encoder of this embodiment consists of a message passing neural network, which given node and edge features is designed to learn a hidden representation of a molecule (i.e., a readout vector). This is done by continuously aggregating neighboring node and edge information through a process called message passing. The readout vector is subsequently split into the mean and variance vectors which serve and as the parameters of the posterior distribution from the sampling. The model may learn a latent distribution that governs molecular properties and provide a decoder which can construct chemically valid molecules from samples of the prior 1505. Latent samples are passed through a sequence of dense layers, after which the two different matrices (node feature matrix, N and edge feature tensor) are used to reconstruct the node feature and edge feature matrices. Keeping with the example described in the paragraph above, these two matrices must have the shapes of (No Atoms, No Node Features) and (No Atoms, No Atoms, No Edge Features) respectively. This may be enforced by using a maximum number of allowed atoms to reconstruct. Further, an additional entry for each of the encoded feature distributions may be allowed, which represents the possibility of No Atom/No Feature. The node and edge feature matrices are compared using an approximate graph matching procedure which looks at atom types, bond types, atom-bond-atom types.

Reinforcement learning may be used in parallel to provide an additional gradient signal, checking that decoded molecules are chemically valid using cheminformatics toolkits. In particular, samples from the prior distribution (N (0,1)) as well as posterior distribution (N (mean, std)) are decoded 1506 and their validity is evaluated 1507. If the cheminformatics toolkit is non-differentiable, then a reward prediction network (a separate MPNN encoder) that is trained to predict the validity of an input graph may be used. Together, these components provide an end to end, fully differentiable framework for training. Other choices for data can be QM9, or any other database that is considered valid.

According to one aspect, in order to make use of more molecules, alternative reconstructability criteria may be used to ensure a chemical similarity threshold instead of perfect reconstruction. For example, encoding and decoding several times and using a molecule if its reconstruction has a chemical similarity above a certain threshold may result in a greater number of reconstructable molecules.

New molecules may also be generated via perturbation, wherein the encodings of the active molecules (i.e., the mean and log(sigma²) values) are taken and Gaussian noise is added to them. A sample from the new (mean, log(sigma²)) values are taken and decoded to derive novel molecules. An important hyperparameter is the magnitude of the Gaussian noise that is added to latent vectors. It is also possible to dynamically adjust the perturbation coefficient, for example, increasing it if the proportion of new molecules is low and decreasing it otherwise.

New molecules may also be generated via interpolation. To generate via interpolation, two random reconstructable molecules are taken, computed together for an interpolation of their latent (mean, log(sigma²)) representations with a random interpolation coefficient, and then decoded to get a new molecule. Generative Adversarial Networks (GANs) excel at interpolation of high dimensional inputs (e.g., images). According to one aspect, the dimension of p(z) corresponds to the dimensionality of the manifold. A method for latent space shaping is as follows: Converge a simple autoencoder on a large z, find the Principal Component Analysis (PCA) which corresponds to the 95th percentile of the “explained variance”, and choose a z within that spectrum (i.e., if the first 17 components of the latent space to represent 95% of the data, choosing z of 24 is a good choice). Now, for high dimensional latent spaces with a Gaussian prior, most points lie within a hyper spherical shell. This is typically the case in multi-dimensional gaussians. To that end, slerp (spherical linear interpolation) interpolation may be used between vectors v1 and v2. Therefore, interpolation is a direct way to explore the space between active molecules.

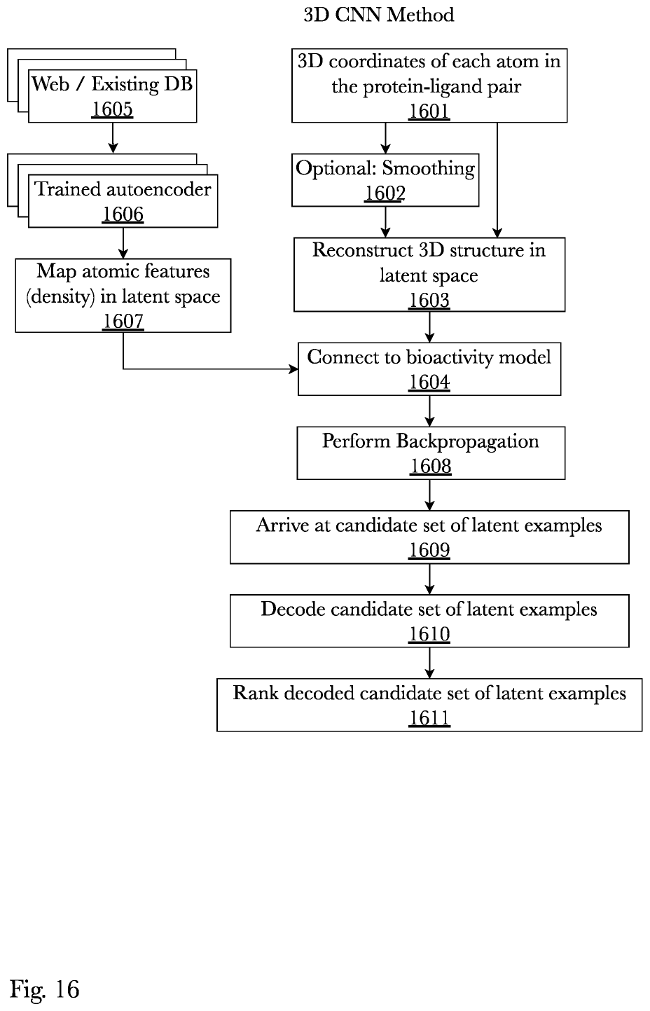

FIG. 16 is a flow diagram illustrating an exemplary method for active example generation using a 3D CNN approach. According to an embodiment of active example generation, a 3-dimensional convolutional neural network (3D CNN) is used in which atom-type densities are reconstructed using a sequence of 3D convolutional layers and dense layers. Since the output atom densities are fully differentiable with respect to the latent space, a trained variational autoencoder (VAE) 1606 may connect to a bioactivity-prediction module 1604 comprising a trained 3D-CNN model with the same kind of atom densities (as output by the autoencoder) as the features, and then optimize the latent space with respect to the bioactivity predictions against one or more receptors. After that, the optimal point in the latent space can be decoded into a molecule with the desired properties.

Three-dimensional coordinates of potential molecules 1601 are used as inputs to a neural network for 3D reconstruction in latent space 1603 (the 3D models of molecules using volumetric pixels called voxels). Underfitting due to data sparsity may be prevented by optional smoothing 1602 depending on the machine learning algorithm used. Existing molecule examples 1605 are used to train one or more autoencoders 1606 whereby the output of the decoder is used to map atomic features such as atom density in latent space 1607 in the bioactivity model 1604, wherein the bioactivity model consists of a sequence of convolutional and fully connected layers. Backpropagation 1608 (or other gradient-aided search) is performed by searching the latent space for regions that optimize the bioactivities of choice thus arriving at a set of latent examples 1609. Decoding 1610 and ranking 1611 each candidate latent example produces the most viable and best-fit to the initial desired parameters.

As an example, a VAE is trained on an enriched molecule data set until optimal reconstruction is achieved. The decoder of the VAE is used as an input to a bioactivity model, wherein the VAE input is a small molecule and the bioactivity module houses a large molecule, i.e., a protein. The behavior and interactions between the molecules are output from the bioactivity model to inform the latent space of the VAE.

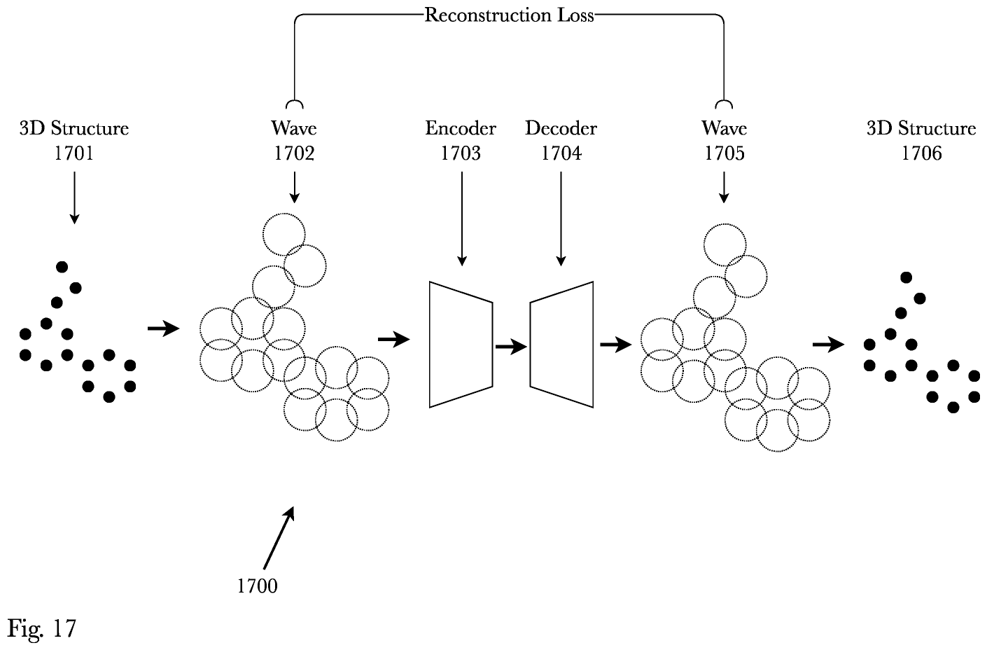

FIG. 17 is a diagram illustrating the training of an autoencoder 1700 of a 3D CNN for active example generation. In a second preferred embodiment, 3D coordinates of the atomic positions of molecules are reconstructed as smoothed (Gaussian blurring as one method) 3D models 1702, 1705 alleviating the underfitting of encoder 1703 and 3D CNN decoder 1704 models due to high data disparity. Wave representations 1702, 1705 allow voxels to convey the same information as the 3D structures 1701, 1706. One exemplary embodiment uses PyTorch, an open-source machine learning library used for applications such as computer vision and natural language processing, and is used to initially train an autoencoder.

Autoencoders 1700 may also be implemented by other programming languages and forks other than PyTorch. Additional embodiments may comprise a complex pipeline involving Generative Adversarial Networks (GANs) and a hybrid between localized non-maximal suppression (NMS) and negative Gaussian sampling (NGS) may be used to perform the mapping of smoothed atom densities to formats used to reconstruct the molecular graph. Furthermore, training autoencoders 1700 on generating active examples by deconvolution is improved by using a GPU (Graphical Processing Unit) rather than a CPU (Central Processing Unit). Using the embodiments as described above, grants input atom densities to generate detailed deconvolutions by varying noise power spectral density and signal-to-noise ratios.

As a detailed example, the generation may be done in the following steps, using any number of programming languages but is described here using the structure of Python, and by creating various functions (where functions are subsets of code that may be called upon to perform an action). The model is initialized with a trained autoencoder and a dataset of active molecules. The latent representations of the active dataset (or their distributions, in the case a variational autoencoder is used) are computed, by learning the latent space, which may comprise one function. This function may also store the statistics of the active dataset reconstructions, to compare with the statistics of the generated data later. A function which generates a set number of datapoints using the chosen generation method is also employed using a flag method within the class instance may control the generation method (e.g. “perturb”, “interp”). Additional parameters for the methods, e.g. the perturbation strength, may be also controlled using instance variables. Another function may be programmed that decodes the generated latent vectors and computes statistics of the generated datasets. These statistics include the validity (percentage of the samples which are valid molecules), novelty (percentage of molecules distinct from the active dataset), and uniqueness (percentage of distinct molecules) of the dataset, as well as the molecular properties, specified in a separate function that computes the properties. Molecular properties may be added or removed to this function at will, without any changes to the rest of the code: summarized statistics and plots are inferred from the molecular properties dictionary. Results may then be summarized in two ways: by printing out the summary of the distributions and generating plots comparing the molecular properties as defined in the computer properties function of the active and generated distributions.

All variables, functions, and preferences are only presented as exemplary and are not to be considered limiting to the invention in any way. Many avenues of training autoencoders or variational autoencoders are known to those in the art by which any number of programming languages, data structures, classes, and functions may be alternatively switched out depending on implementation and desired use.

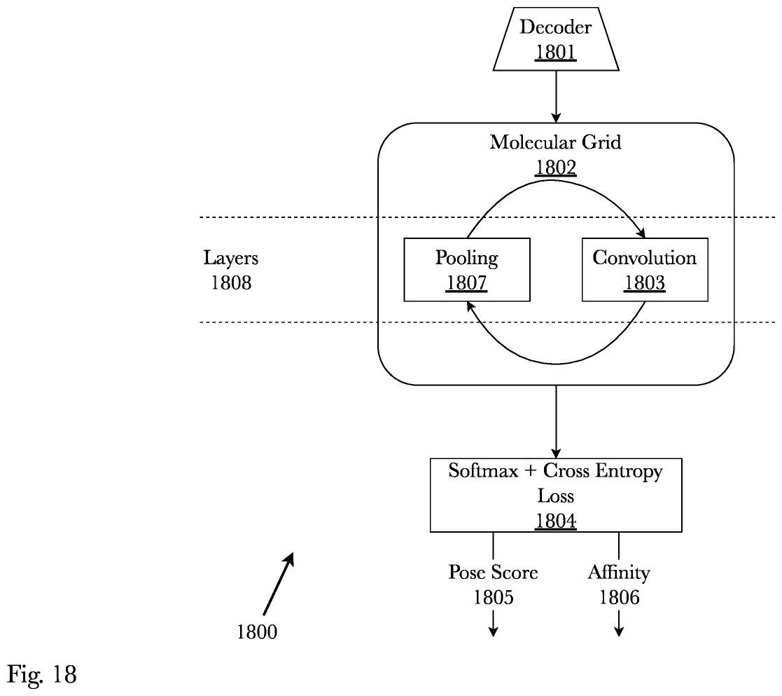

FIG. 18 is a diagram illustrating the interfacing of the decoder to the 3D-CNN bioactivity prediction model 1800. During training of the neural network machine learning model with inputs of a 3D grid 1802 of Gaussian-like atom type densities, the weights are iteratively modified in order to minimize the losses 1804, which is some measure of the goodness of fit of the model outputs to the training data. In an embodiment, the procedure is performed using some variation of gradient descent, where the changes applied to each weight during the update step are proportional in some way to the gradient of the loss with respect to the weight in question. The calculation of these gradients is often referred to as backpropagation, as the gradients of the loss with respect to a weight (n+1) layers removed from the model output depend, as per the chain rule, only on the gradients of the weights in the layers (0, . . . , n) 1808 away from the model output 1805, 1806, and they are therefore calculated first in the layer closest to the model output and loss, the results of which are used both to update the weights and to calculate the gradients of the loss 1804 with respect to weights further back in the model.

Layers 1808 may perform a function with some parameters and some inputs, as long as the computation performed by a layer 1807/1803 has an analytic derivative of the output with respect to the layer parameters (the faster to compute, the better) These parameters may then be learned with backpropagation. The significance of using voxelated atom-features as inputs to a bioactivity model (as in the case of a 3D CNN) is that the loss can be differentiated not only with respect to the layer weights, but also with respect to the input atom features.

According to one aspect, various cheminformatics libraries may be used as a learned force-field for docking simulations, which perform gradient descent of the ligand atomic coordinates with respect to the binding affinity 1806 and pose score 1805 (the model outputs). This requires the task of optimizing the model loss with respect to the input features, subject to the constraints imposed upon the molecule by physics (i.e., the conventional intramolecular forces caused for example by bond stretches still apply and constrain the molecule to remain the same molecule). Attempting to minimize the loss 1804 directly with respect to the input features without such constraints may end up with atom densities that do not correspond to realistic molecules. To avoid this, one embodiment uses an autoencoder that encodes/decodes from/to the input representation of the bioactivity model, as the compression of chemical structures to a smaller latent space, which produces only valid molecules for any reasonable point in the latent space. Therefore, the optimization is performed with respect to the values of the latent vector, then the optima reached corresponds to real molecules.

Application of this comprises replacing the input of a trained bioactivity model with a decoder 1801 portion of a trained 3D CNN autoencoder, which effectively ‘lengthens’ the network by however many layers 1808 are contained within this decoder. In the case of a 3D CNN bioactivity model, the 3D CNN autoencoder would thus form the input of the combined trained models. This embodiment allows both differentiable representations which also have an easily decodable many-to-one mapping to real molecules since the latent space encodes the 3D structure of a particular rotation and translation of a particular conformation of a certain molecule, therefore many latent points can decode to the same molecule but with different arrangements in space. The derivative of the loss with respect to the atom density in a voxel allows for backpropagation of the gradients all the way through to the latent space, where optimization may be performed on the model output(s) 1805, 1806 with respect to, not the weights, but the latent vector values.

Following this optimization, the obtained minima can be decoded back into a real molecule by taking the decoder output and transforming the atom-densities into the best-matching molecular structure. During optimization of the latent space, it is likely that some constraints must be applied to the latent space to avoid ending up in areas that decode to nonsensical atom densities.

FIG. 20 is a block diagram of an overall model architecture of a system for de novo drug discovery according to one embodiment. The exemplary model described herein is a variational autoencoder (VAE) 2001-2007 together with a reinforcement learning (RL) component 2008-2010 for a graph-based approach. The aim of said model is to learn a latent distribution that governs molecular properties and provide a decoder 2004, 2009 which can construct chemically valid molecules from samples of the prior. With reinforcement learning 2008-2010 to provide an additional gradient signal, decoded molecules may be checked for chemical validity. Samples from the prior distribution as well as posterior distribution are decoded, and their validity is evaluated. As most cheminformatics toolkits chemical validity checking process is not differentiable, a reward prediction network (a separate MPNN encoder 2011) must be used which is trained to predict the validity of input graph 2001. Together, these components provide an end to end, fully differentiable framework for training.

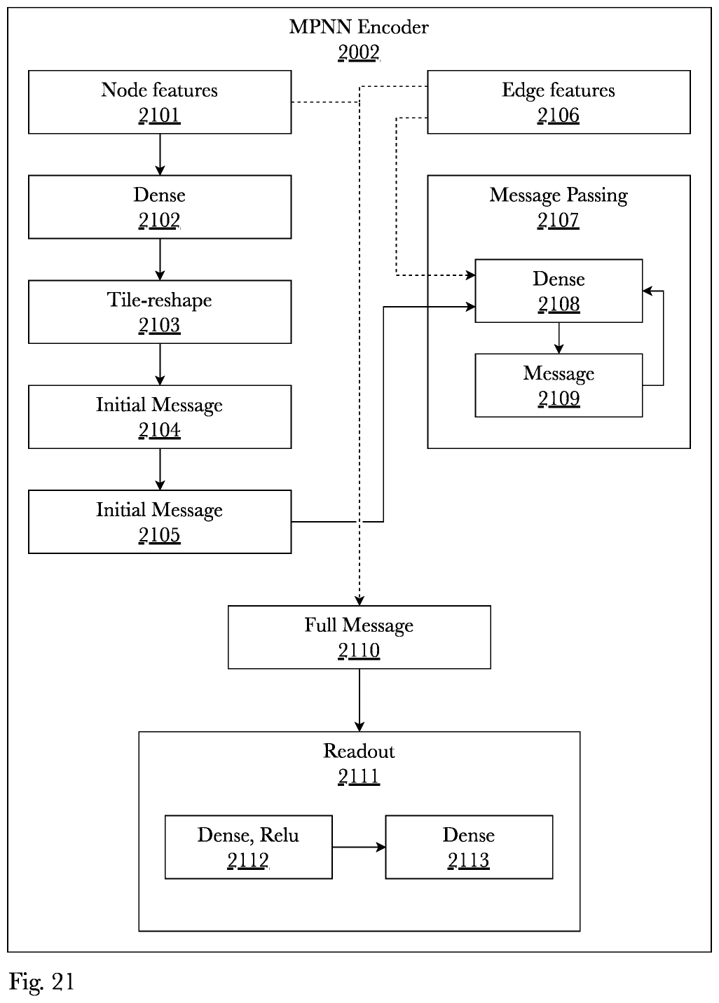

FIG. 21 is a block diagram of a model architecture of a MPNN encoder 2002 for de novo drug discovery according to one embodiment. MPNN Encoder 2002 consists of given node 2101 and edge features 2106 that are input to dense layers 2102, reshaped 2103, summed 2104, concatenated 2105, and circulated within a message passing neural network 2107-2110, which learns a hidden representation of a molecule (Readout vector 2111). This is done by continuously aggregating neighboring node 2101 and edge 2106 information through a process called message passing 2107. Readout vector is subsequently split in to the mean and variance vectors 2112, 2113 which serve and as the parameters of the posterior distribution from which the latent samples 2302 are sampled.

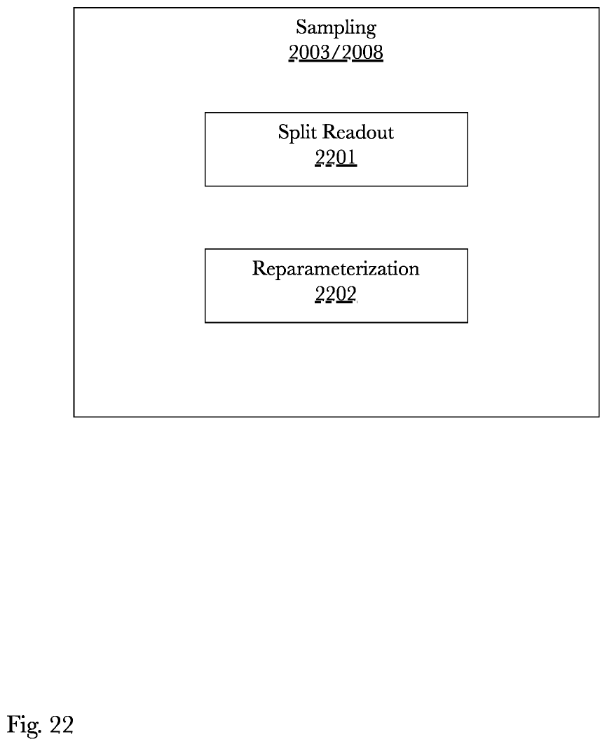

FIG. 22 is a block diagram of a model architecture of a Sampling module 2003/2008 for de novo drug discovery according to one embodiment. The sampling module comprises a split readout function 2201 that produces the mean and log(sigma²) of the batch. A reparameterization function 2202 is used to get a differentiable sampling procedure and a sample of N (mean, std) using a known property of the Gaussian distribution. N (mean, std) is equal to N (0, 1) times sigma plus the mean.

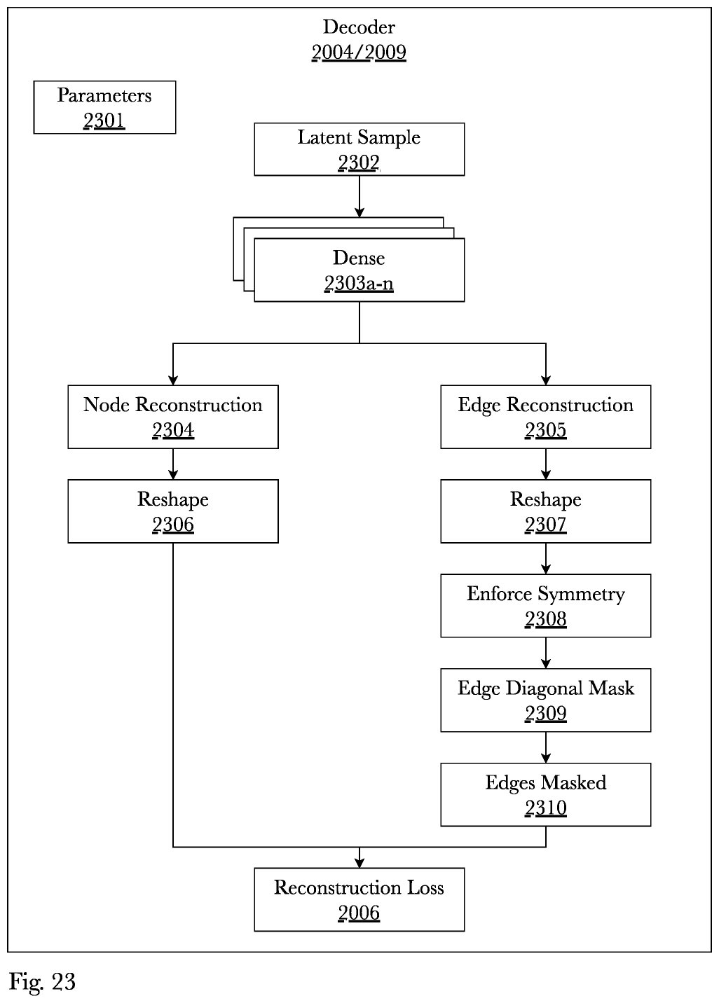

FIG. 23 is a block diagram of a model architecture of a decoder 2004/2009 for de novo drug discovery according to one embodiment. A decoder 2004/2009 with parameters 2301 for the maximum number of atoms to generate along with node and edge size is used to formulate the reconstruction loss 2006. Latent samples 2302 are passed through a sequence of dense layers 2303a-n and subsequently processed via two different matrices to reconstruct node feature 2304 and edge feature 2305 matrices. Shape functions 2306, 2307 ensure the shapes of (No Atoms, No Node Features) and (No Atoms, No Atoms, No Edge Features) respectively. Currently this is enforced by using a maximum number of allowed atoms to reconstruct. Further, an additional entry for each of the encoded feature distributions is performed, which represents the possibility of No Atom/No Feature 2308-2310. Finally, the node and edge feature matrices are compared using an approximate graph matching procedure 2006 which looks at atom types, bond types, atom-bond-atom types.

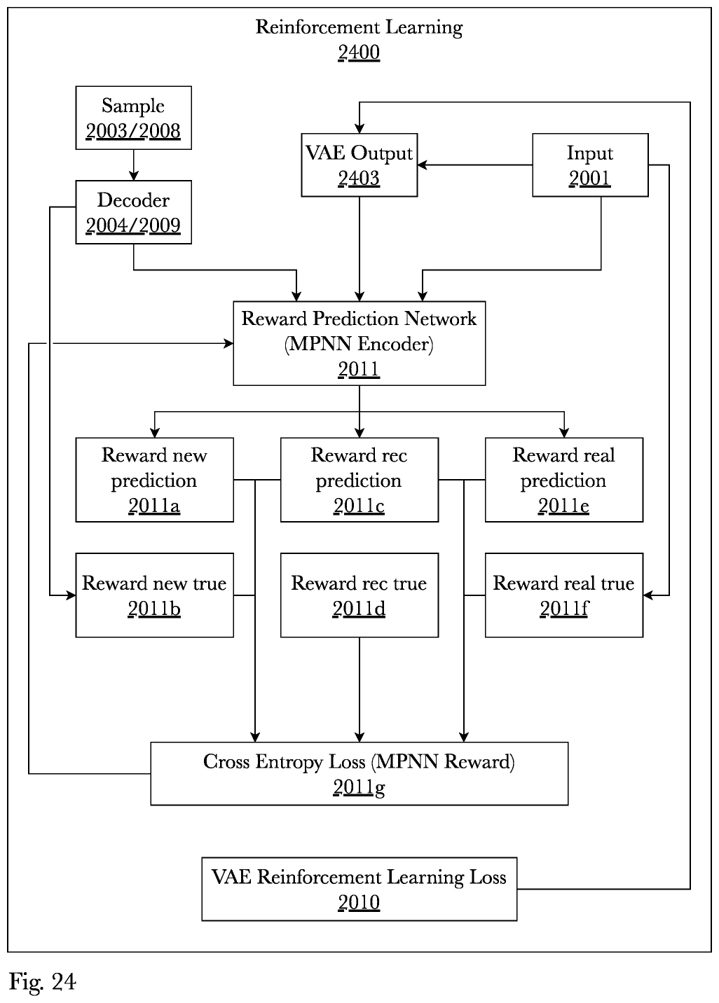

FIG. 24 is a block diagram of a model architecture for reinforcement learning 2400 for de novo drug discovery according to one embodiment. The reinforcement learning 2400 as also shown in FIG. 20, comprises samples 2003/2008 and nodes and edges that inform a reward prediction network 2011. The reward prediction network 2011 receives a batch of latent examples from the decoders 2004/2009, nodes and edges from the VAE output 2403 and the input 2001, where the output of the VAE 2403 is made up of reconstructions of received nodes and edges from the input 2001. The MPNN encoder 2011 is trained to predict rewards 2011a-f given the nodes and edges. Cross entropy loss 2011g is the sum of each of the individual reward combinations 2011a-f and is backpropagated through the reward prediction network 2011, while the VAE RL loss 2010 is fed back into the VAE output 2403.

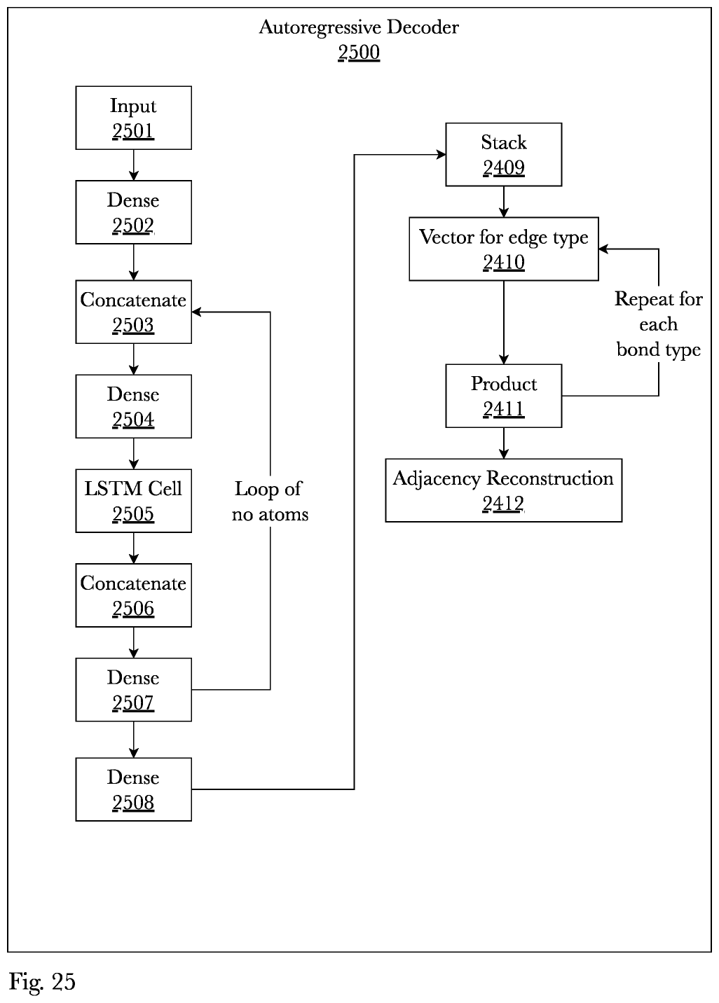

FIG. 25 is a block diagram of a model architecture of an autoregressive decoder 2500 for de novo drug discovery according to one embodiment. Latent vectors of size dimension z are inputs 2501 to the autoregression decoder 2500 and subsequently calculated into dense layers 2502 where their dimensions may be expanded. A concatenation function 2503 precedes a second dense layer 2504 where pre-LSTM feature extraction occurs. After the LSTM cell function 2505, which corresponds to the LSTM recurrence operation, another concatenation occurs 2506 before a third dense layer 2507 extracts nonlinear features. The loop between the third dense layer 2507 and the first concatenation has no atoms. The fourth dense layer 2508 processes atom node features for the stack 2409 to begin node reconstruction. For each bond type a vector for the edge type is created 2410 where the product 2411 outputs probable bond types between nodes. Lastly, adjacency reconstruction 2412 is modeled by a set of edge-specific factors, (e.g., logistic sigmoid function, the corresponding diagonal vector matrix) which are learned parameters.

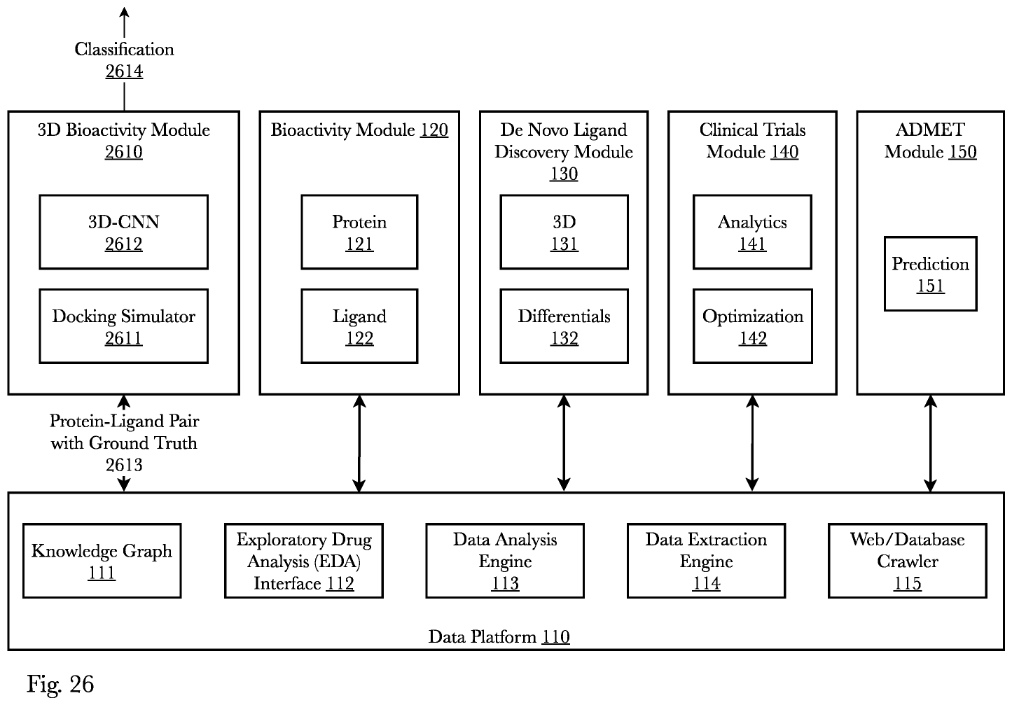

FIG. 26 is a block diagram of an exemplary system architecture for a 3D Bioactivity platform. According to one embodiment, a 3D bioactivity module 2610, comprising a docking simulator 2611 and a 3D-CNN 2612 may be incorporated into the system described in FIG. 1 containing elements 110-151. A data platform 110 scrapes empirical lab results in the form of protein-ligand pairs with a ground-truth state 2613 from public databases that is then used in a docking simulator 2611 to produce a data set for which to train a three-dimensional convolutional neural network (3D-CNN 2612) classifier, which as disclosed herein is a model that can classify a given input of a certain protein-ligand pair is active or inactive and whether or not the pose is correct 2614. A key feature of the 3D-CNN bioactivity module 2610 as disclosed herein, is the ability to produce visualizations of the interactions in the input that are vital to the active/inactive classifications in a more interpretable manner than a FASTA-based model currently used in the art. The output incorporates gradients relating to the binding affinity of specific atoms that a user may use to understand where the model was most attentive and would further provide an explanation why specific molecules are bioactive and why certain molecules are not and to identify the important residues of the binding site. Once the residues are identified, sequence-based similarities algorithms may identify similar motifs in other proteins from the same family or in completely novel proteins relating to that ligand interaction. Furthermore, the 3D-CNN model disclosed herein improves upon current art by penalizing the model for incorrect docking, thus leading to a three class classification 2614: active, inactive, and incorrect docking.

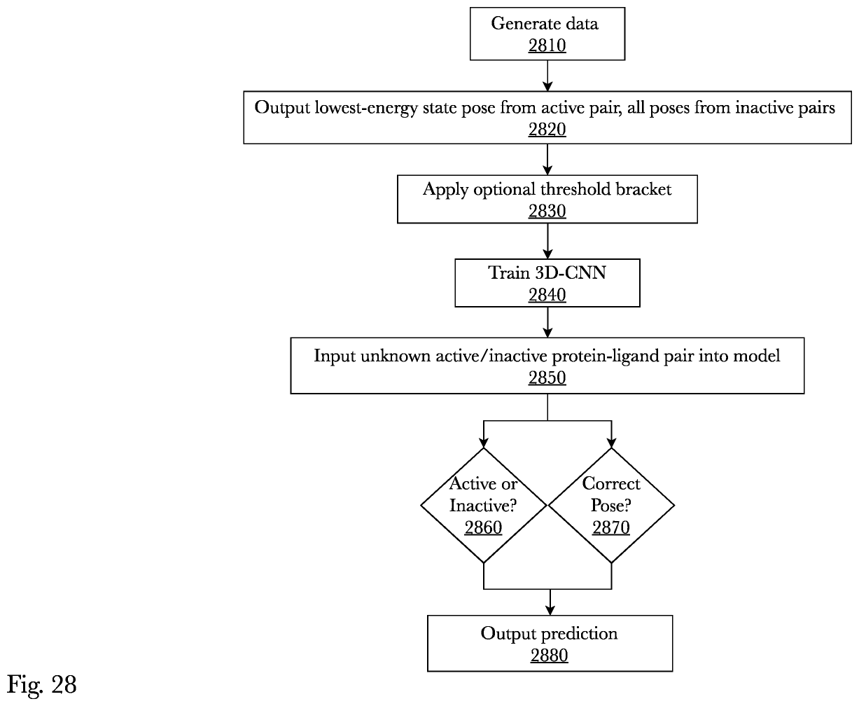

FIG. 28 is a flow diagram illustrating an exemplary method for classifying protein-ligand pairs using a 3D Bioactivity platform. Data is generated 2810 from lab-based empirical evidence which constitutes protein-ligand pairs and their ground-truth state. That data is sent to a docking simulation whereby energy states of the input poses are output along with a classification of active/inactive—from the lab data 2820. The training data presents a choice of a threshold bracket 2830. The threshold bracket is a trade-off between the average information contained in each datapoint, and the sheer quantity of data, assuming that datapoints with more extreme inactive/active IC₅₀ values are indeed more typical of the kind of interactions that determine whether or not a protein-ligand pair is active or inactive. In the case of the 3D-model, using the dataset with no threshold performs consistently better across most metrics. The channels used for the data set are hydrophobic, hydrogen-bond donor or acceptor, aromatic, positive or negative ionizable, metallic and total excluded volume. Regardless of the choice of threshold, the data is then used to train a 3D-CNN to know the classification of a molecule regarding activation and pose propriety 2840. The 3D bioactivity platform then receives an unknown molecule 2850 that is fed into the model to determine its classifications 2860/2870. The prediction is output 2880, and in some embodiments, may be used in backpropagation to further inform the model.

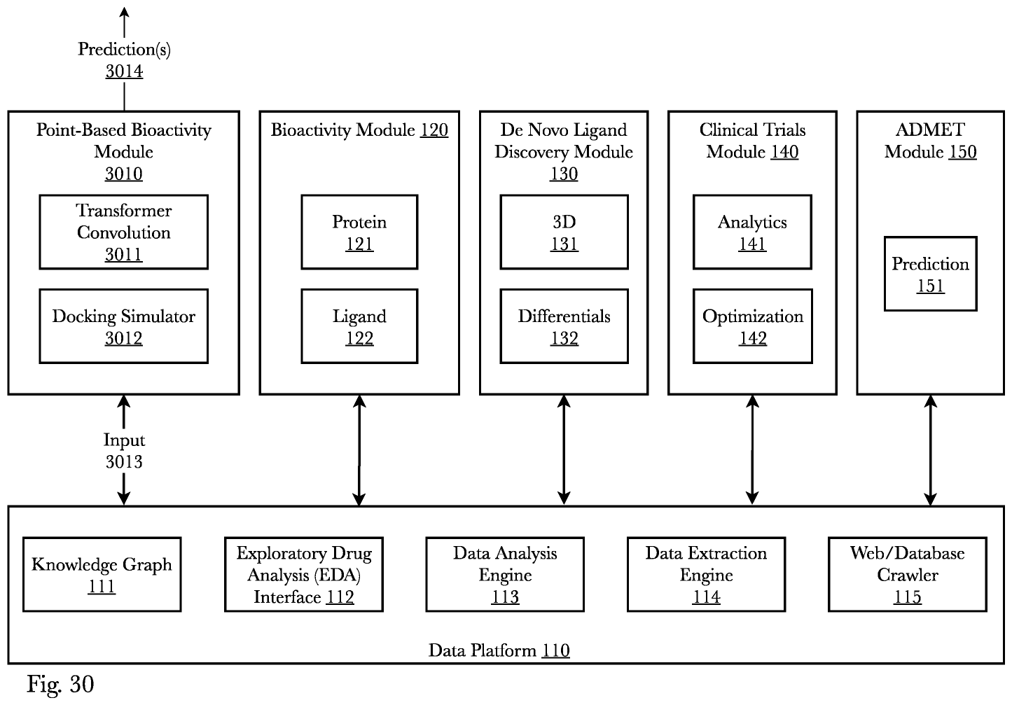

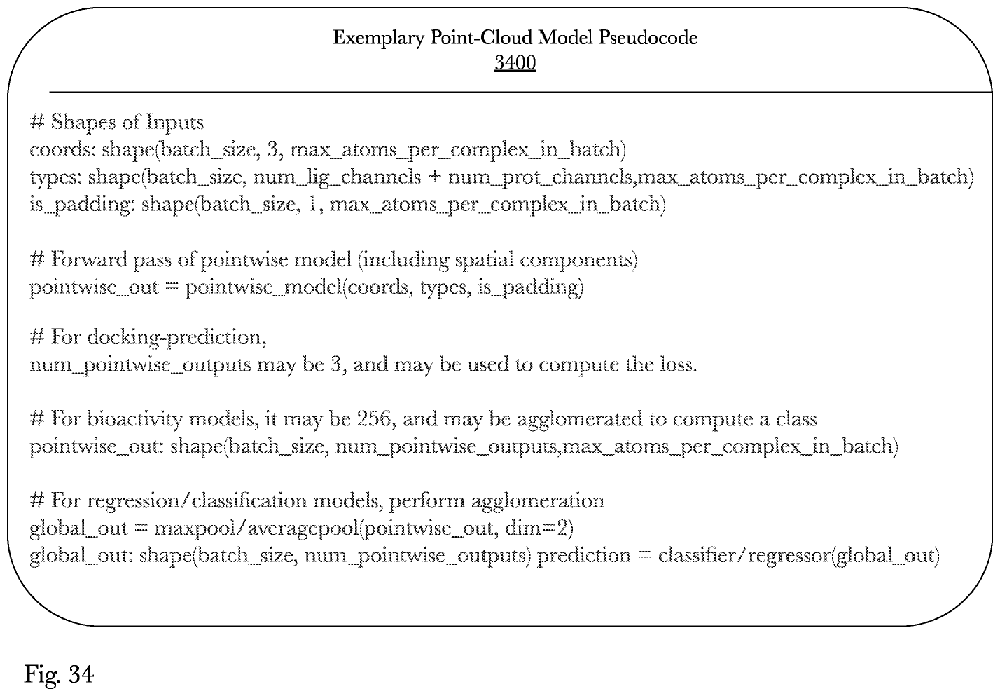

FIG. 30 is a block diagram of an exemplary system architecture for a Point-Cloud Bioactivity platform. Instead of representing protein-ligand complexes as voxel grids containing atom densities as in other embodiments disclosed herein, this embodiment represents protein-ligand complexes as atoms with associated coordinates, i.e., point-cloud models. Point cloud models allow for pointwise operations on these coordinates (and in some cases operations that capture spatial relationships between points), and, depending on the task, agglomerate these into either a single representation that can be used for a classification or regression task, or to perform a pointwise regression or classification task on the set of points. An exemplary pseudocode implementation 3400 is presented in FIG. 34.

According to one embodiment, a point-cloud bioactivity module 3010, comprising a docking simulator 3012 and one or more transformer convolution algorithms 3010 may be incorporated into the system described in FIG. 1 or in FIG. 26, both containing elements 110-151. The point-based bioactivity module 3010 comprises two main operating states with two more substrates in each main operating state. The first main operating state is a training state from which a model for the second main operating state is produced. The second main operating state is a platform for which users may query one or more protein-ligand pairs and receive a bioactivity prediction, the prediction generated from the trained model achieved from the first main operating state. For both main operating states, there are two modes of operation: Coupled and decoupled. Whether training or querying, the point-based bioactivity module 3010 may use the docking simulator 3012 or not. In a specific coupled case, the docking simulator 3012 may be used initially but if the results are incongruent, the simulation failed, or the results are otherwise undesirable, the point-based bioactivity module 3010 will default to a decoupled operating state. The input 3013 to the point-based bioactivity module 3010, whether training or querying, are molecular structure files. These molecular structure files are either in the form of empirical lab results retrieved and scraped via the data platform 110 for the training mode of operation, or submitted by a user for the querying mode of operation. The user may submit a protein file, or a protein file with an alternative ligand file, or coordinates of the binding pocket, or other alternative protein-ligand file configurations. The option to use a decoupled or coupled state—whether the protein-ligand pose propriety is known or predicted—may be decided depending on the task at hand. Given specific time or computing constraints, the user may manually, or the point-based bioactivity module 3010 may automatically choose a decoupled operating state, bypassing the docking simulator to save time or computing resources.

During the training operating state, predictions 3014 from the transformer convolution module(s) 3011 comprise whether the protein-ligand pair generated matches closely enough the crystal structure of the ground-truth pair, and the bioactivity. Typically, but not limited to, a threshold of 2 angstroms is used to determine the crystalline structure similarity. The crystal structure similarity may also be used to decide whether to penalize the model for predicting too high a bioactivity if the crystal-structure comparison does not meet the 2-angstrom threshold.

During the querying operating mode, a bioactivity prediction is output, combined from the regression output and the classification of active/inactive-ness. Further, a 3D dimensional model of importances is generated as well. The output predictions 3014, described above are merely exemplary, and it is to be understood that in either training or querying mode, all five outputs (active, inactive, crystal-like, not crystal-like, and regression) or any combination thereof may be used.

Further, according to various embodiments, model ensembling, or the use and combination of various machine learning models is anticipated. This means, in addition to the transformer convolution system and method described in this embodiment, other machine learning models may be integrated within, replaced by other models, and otherwise combined in such a way to enhance the prediction of bioactivity.

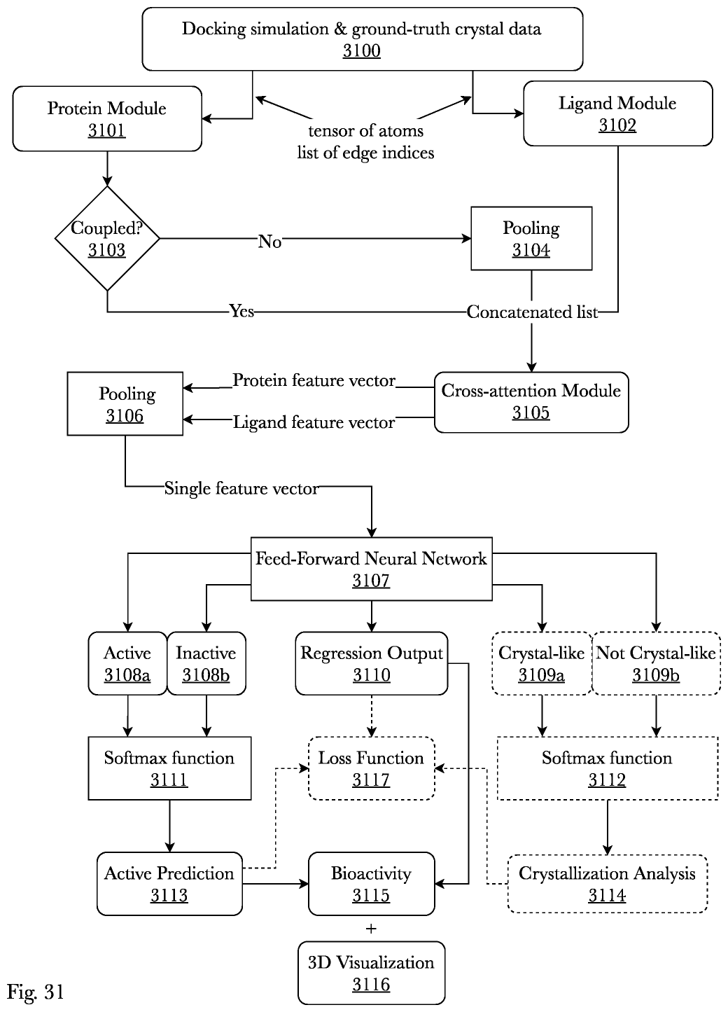

FIG. 31 is a block diagram of an exemplary model architecture for a Point-Cloud Bioactivity platform. The output of the model comprises one or more predictions (e.g., active or inactive, crystal structure similarity, regression output, and bioactivity). The outputs are task dependent, wherein the output may contain one or more of the classifications, regressions, a bioactivity value, or some combination thereof. The values of regression or bioactivity may take any form desirable to the user; however, a few exemplary values comprise units of −log₁₀(IC₅₀), or −log₁₀(Ki), or the negative log base ten of any other bioactivity metric, which again is merely exemplary.

As described in FIG. 30, molecular structure files are used as the inputs 3100, and come from a variety of sources. As one example, a user may upload a structural file of a protein which comprises an ID from which the bioactivity platform downloads a corresponding molecular structure file. Then the user informs the system where in the pocket the ligand lies, or may upload a file of the ligand in the binding pocket, or just supply the molecular structure of a ligand. These inputs are fed as into two independent modules 3101/3102, one for the protein 3101 and a separate one for the ligand 3102. Because a point-cloud model is a graph with no edges, edges for the protein model, i.e., the protein module 3101, are generated by determining the proximity of all the atoms within a certain distance to the ligand or binding pocket. The ligand submodule 3102 creates edges for the ligand model from the information contained in the molecular structure file comprising bonds, e.g., single bonds, double bonds, aromatic bonds, etc. For decoupled protein-ligand pairs, i.e., protein-ligand pairs where docking simulations failed or are ligand-binding pocket conformations otherwise not available 3103, pooling (Max, weighted, etc.) 3104 is performed on the protein, creating a collapsed vector of protein atoms. For example, only protein atoms from the binding site are kept for decoupled pairs, while protein atoms within 4 angstroms of the ligand are kept for a coupled model.

The output of the protein and ligand modules 3105 also includes the combined protein-ligand complex of coupled molecules via docking simulations. In other words, the input to the cross attention module 3105 is a concatenated atom list of the protein and ligand, wherein the edge list contains only edges where one atom is a protein, and one atom is a ligand atom. Furthermore, the cross attention module 3105 restricts attention between protein and ligand atoms in close proximity, ergo, the model can only learn from the actual interactions, whereas without this restriction, there is no coupling between protein and ligand. The vector output of the cross-attention model 3105 is pooled 3106 into a single feature vector that feeds into a feed-forward neural network 3107.

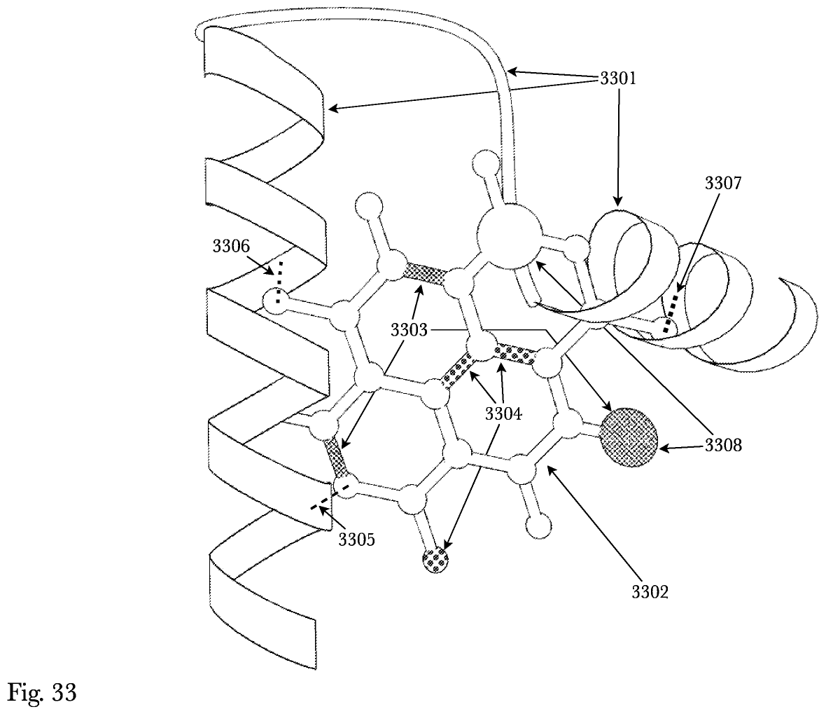

One set of outputs is a crystal-structure similarity analysis 3114, which is comprised from two output nodes 3109a-b that are sent through a SoftMax function 3112, and predict whether the protein-ligand pair in question is similar enough to the ground truth crystal structure (typically within a 2 angstroms threshold) 3111. Typically, the crystallization analysis 3114 is only used for training, however output during a user query is anticipated. Another output comprises a SoftMax function 3111 containing two output nodes of the active/inactive prediction 3108a-b of the protein-ligand pair in question and producing a prediction 3113 of that active/inactive status. The regression output 3110 and active-ness prediction 3113 inform the bioactivity prediction value 3115. A user query may return in addition to a bioactivity prediction 3115, a 3D visualization 3116 of the queried protein-ligand pair with various information about the importances as laid out in FIG. 33.

During training of the model, a loss function 3117 is used and is configured to penalize the model if the model predicts too high a bioactivity for a non-crystal-like structured protein-ligand pair, but is not penalized for predicting too low a bioactivity, while also simultaneously trained on the classification task. At prediction time, the model may use the crystal structure probability to decide whether to take bioactivity reading or discard it as inaccurate. From there more docking poses may be generated until a likely crystal structure is found.

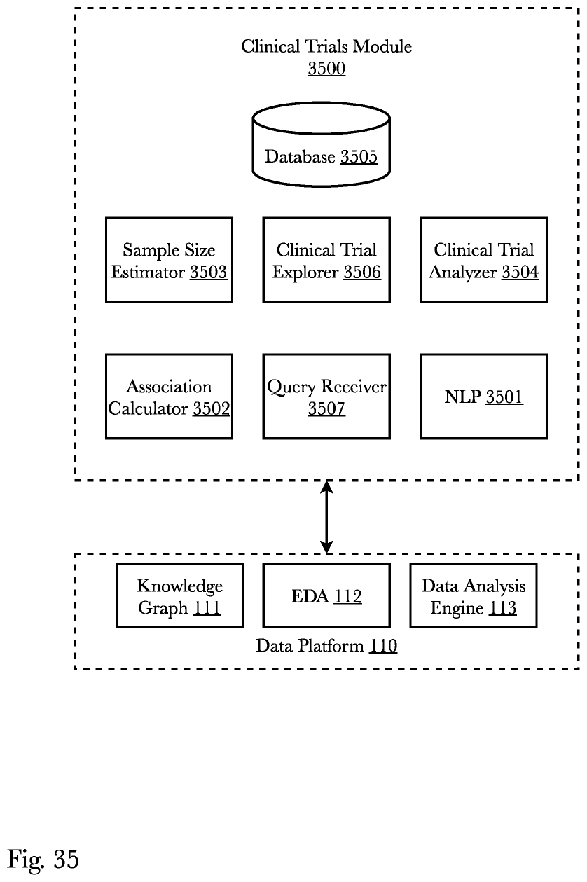

FIG. 35 is a block diagram illustrating an exemplary system architecture for biomarker-outcome prediction and clinical trial exploration 3500, according to an embodiment. The system 3500 leverages the data platform 110 components, knowledge graph 111, exploratory drug analysis (EDA) interface 112, and data analysis engine 113 to provide analytical, discovery, and prediction capabilities based on a large corpus of published clinical trial and assay information. Using the system, users can research a large database of clinical trials by entering queries through the EDA interface 112 which is parsed by a query receiver 3507. Query receiver 3507 receives a query from the EDA 112 and directs the query details to the appropriate end point. One type of query that may be received and processed by the system 3500 is a biomarker search query which causes the system to return a list of papers which link the input biomarker to a plurality of outcomes, forming one or more biomarker-outcome pairs. In this use-case, the query receiver 3507 would send the input biomarker to the NLP—using tool 3501 which would begin parsing all available clinical trial publications to identify and then return all publications that mention the input biomarker. Additionally, biomarkers may be empirically linked to some outcome. The association calculator 3502 utilizes the NLP-using tool 3501 to calculate the co-occurrence of biomarker-outcome pairs to generate an association rank which may represent the biological or medical relationship between biomarker and outcome. Calculated association scores may be persisted in a database 3505. Another type of query that may be received and processed by the system 3500 is a sample size query. A sample size query may consist of input terms which describe the goal, methodology, and design of a potential clinical trial in order to provide context for the sample size estimator 3503 to calculate a sample size. Sample size estimator 3503 may use prior information sampled from similar historical clinical trial data based on input terms.

When the data platform 110 ingests a clinical trial publication it may be sent to a natural language processing pipeline 3501 which scrapes a publication for information that pertains to custom fields of a standard clinical trial data model. Once a publication has been fully scraped, the standard data model is persisted to a database 3505 and the information contained within the standard data model is added to the knowledge graph 111. A clinical trial explorer 3506 may pull information about each clinical trial from its standard data model as well as from a subset of the knowledge graph 111 in order to create a navigable user interface for clinical trial exploration. In one embodiment, the clinical trial explorer 3506 may create a global map of research centers which have published medical literature pertaining to clinical trials, assays, or research studies using the geolocation data scraped during the data ingestion process. The global map would allow a user to navigate and explore clinical trials associated with each research center. For example, a research center may be denoted with a star on a map and a user could hover over the star to get a quick snapshot of the research center, the snapshot may include information such as, but not limited, the research center name, total number of publications, research field (i.e., drug research, genetic research, etc.), and most recent publication. Clicking on the star would take the user to a separate page populated with the abstracts of each published paper and a link that directs the user to the original paper. The separate page may also include for each paper a list of any biomarkers and biomarker-outcome pairs discussed within each paper. In other embodiments, the clinical trial explorer 3506 may facilitate clinical trial exploration via a navigable graph interface.

The clinical trial analyzer 3504 may utilize the data analysis engine 113 and knowledge graph 111 to provide explanatory capabilities that provide deeper context between a biomarker and an outcome. The knowledge graph 111 contains a massive amount of data spanning categories such as diseases, proteins, molecules, assays, clinical trials, and genetic information all collated from a large plurality of medical literature and research databases which may be used to provide more insight into biomarker-outcome relationships. A clinical trial may provide a relational link between a biomarker and an outcome, as an example consider the biomarker chloride: an increased chloride level in hypochloremia is associated with decreased mortality in patients with severe sepsis or septic shock. In this example the biomarker is associated with the adverse event (outcome) of mortality and with the outcome sepsis/septic shock. The clinical trial analyzer 3504 may scan the knowledge graph 111 for sepsis or septic shock and then find what biological process associated with sepsis. It is known that sepsis occurs when chemicals released in the bloodstream to fight an infection trigger inflammation throughout the body which can cause a host of changes that can damage various organ systems. For example, the analyzer 3504 may be able to identify the molecular profile of the chemicals released into the blood stream and then make a connection between the molecules and chloride that provide a richer context between chloride and sepsis. In this way the biomarker-outcome prediction and clinical trial exploration system 3500 may provide explanatory capabilities to add deeper understanding between a biomarker and outcome.

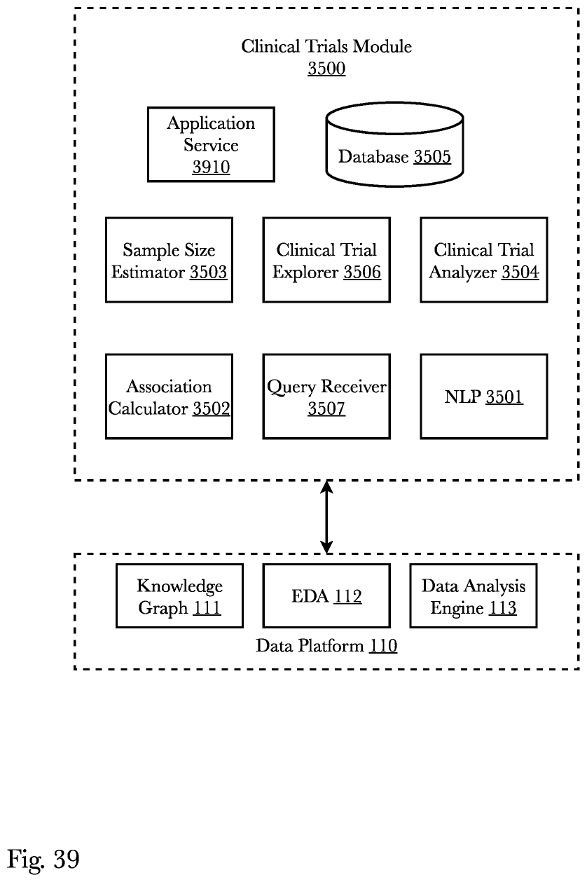

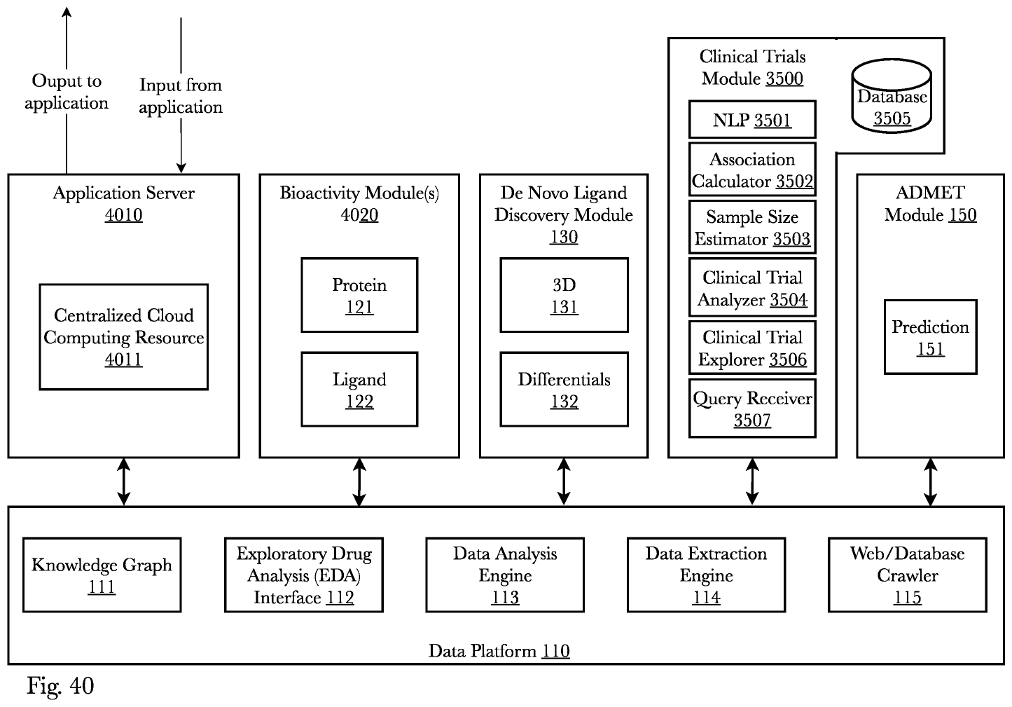

FIG. 39 is a block diagram illustrating an exemplary system architecture for biomarker-outcome prediction and clinical trial exploration for edge computing. This diagram illustrates a modification to the system described in FIG. 35, wherein the modification is an application service 3910 that functions as a centralized cloud computing resource for edge devices, wherein the edge devices are running a mobile application comprising an independent machine learning model for clinical trial analysis that updates the centralized cloud computing machine learning models, which in turn push out updated machine learning models and/or their parameters to the edge devices. This embodiment illustrates a standalone clinical trials module 3500 that may be used for edge computing, as opposed to the next figure (FIG. 40) which implements and application server 4010 in order to provide services from the other modules 4020, 110, 130, 150 to the edge devices.

FIG. 40 is a block diagram illustrating an exemplary overall system architecture for a pharmaceutical research system for edge computing. A pharmaceutical research system comprising one or more bioactivity modules 4020 and components 110-151, 3500-3507 now also comprises an application server 4010 which acts as a centralized cloud computing resource 4011, according to various embodiments. The one or more bioactivity modules 4020, the clinical trials module 3500 and respective components 3501-3507, and other components 110-151, are as previously described in FIG. 1, FIG. 26, FIG. 30, and FIG. 35, but are not limited to those figures as other figures herein also disclose various functions and features of various pharmaceutical research system embodiments. Furthermore, various embodiments of a pharmaceutical research system comprising an application server 4010 need not have every component (4020/3500-3507/110-151) illustrated here. This diagram is merely an example of integrating together all herein-disclosed aspects of a pharmaceutical research system and the application server 4010 into one system.

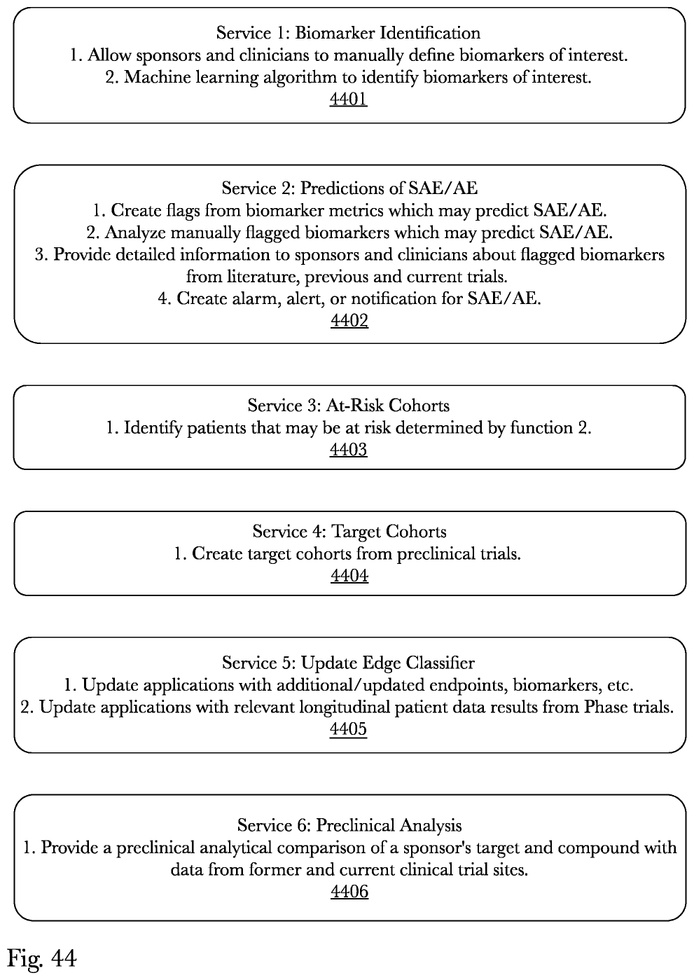

According to one embodiment, an application server 4010 serves as a centralized cloud computing resource 4011 for edge devices running a mobile application for clinical trial analysis. This embodiment uses one or more of the machine learning aspects disclosed within this specification, notably at least one or more of the aspects outlined in FIG. 44, to better make predictions and analysis about clinical trials. While the clinical trials module 3500 nearly covers all the aforementioned aspects, other features (bioactivity modules 4020, ADMET module 150, etc.) of a pharmaceutical research system may be used by edge devices as desired, such as in this disclosed in this embodiment.

Edge devices in the possession of sponsors, clinicians, and patients utilize a mobile application which may or may not comprise a machine learning model. Some edge devices may rely on local edge-AI hardware or simply relay information to a cloud computer 4011. However, it is likely that most edge devices utilized by sponsors and sites are smart phones, tablets, laptops, and desktops all capable of running the mobile application with a machine learning model. The model may be a classifier or another type of machine learning model. The machine learning model is logically part of a larger machine learning scheme within a pharmaceutical research system, wherein the edge models send information out to one or more cloud-based machine learning models for more computationally intensive tasks. Subsequently, cloud-based machine learning models may push out one or more machine learning models via an application server 4010 to the edge devices, in such a case where a new edge device is initialized, the edge device model is outdated or erred in some way, or just for periodic synchronization of edge devices across multiple sites for a clinical trial.

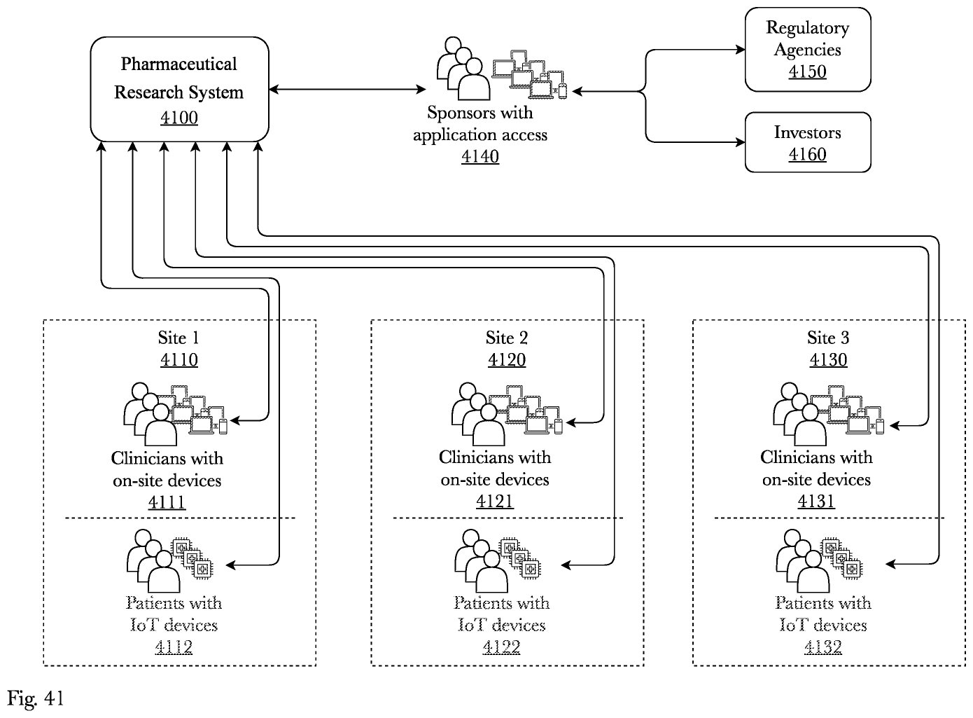

Edge tasks may comprise autonomous biomarker identification, discovering trends, indications, and values in biomarkers that may predict SAEs and AEs, and identify various cohorts of patients. Whereas cloud tasks—the more computationally intensive, but not strictly computationally intensive tasks—may comprise autonomous biomarker identification across multiple sites or one site, discovering trends, indications, and values in biomarkers that may predict SAEs and AEs across multiple sites or one site, identify various cohorts of patients across multiple sites or one site, create detailed reports about biomarkers, and create analytical comparisons of a sponsor's target and compound with data for former and current clinical trial sites by variables such as: target, drug, endpoints, SAEs and AEs. Those skilled in the art will recognize that some tasks are better suited for one type of machine learning model than another. For example, as in the clinical trials module 3500, NLP may be used for creating the detailed biomarker reports, and on the other hand, the ADMET module 150 may use a message-passing neural network for predicting pharmacological properties (as disclosed in co-pending application Ser. No. 17,175,832) for assisting in SAE/AE predictions. Additional features and functions of edge devices, edge device mobile applications, edge device mobile application machine learning models, and cloud computing resources follow in FIG. 41, while specific machine learning methods to build one or more machine learning models is found in FIG. 44.

Implementation of machine learning on edge devices may be accomplished via various edge-computing platforms in place such as TENSORFLOW, NVIDIA JETSON, and other such lightweight machine learning frameworks.

FIG. 41 is a block diagram illustrating an exemplary system architecture for an embodiment of a pharmaceutical research system 4100 utilizing edge devices. This diagram illustrates the utility and improved communications of data packets within a clinical trial as well as the edge-computing architecture. A pharmaceutical research system 4100 may be cloud-based as in this embodiment, or may be hosted on an intranet or other network type depending on the desired security and privacy restrictions. This embodiment exemplifies the use of edge devices with a mobile application used by sponsors 4140 and clinicians 4111, 4121, 4131, wherein the mobile application comprises a set of features and machine learning adapted to clinical trial analysis and communication with a cloud-based pharmaceutical research system 4100, the features and machine learning details having already been disclosed and additional details to be explained henceforth. Additionally, patients may be equipped with IoT devices 4112, 4122, 4132 which measure certain biomarkers of a patient. Anticipated aspects also include the use of active and passive IoT devices, sensors, lab equipment, medical measuring devices, imaging devices, lab results, and other such biomarker measuring means typically available to clinical trial sites and the medical industry as a whole.

Current clinical trials use a variety of means to transmit information including email, postal mail, facsimile, online databases, etc. These means, while some faster than others, also suffer from the need for human interpretation and human error. As presented here, this diagram illustrates how edge devices complete with machine learning algorithms and models compliment a larger machine learning infrastructure in the cloud, and provide immediate, real-time, or near real-time analysis of all the information from a clinical trail and does not depend on a human to piece-meal the information together from heterogenous sources.

As an example, consider that this diagram represents one clinical trial. This clinical trial has three geographically disperse sites 4110, 4120, 4130, three separate teams of clinicians 4111, 4121, 4131, and three sub-cohorts of patients 4112, 4122, 4132. Prior to the initialization of a clinical trial, pharma companies 4160 and sponsors 4140 may use a pharmaceutical research system 4100 to de-risk investment decisions for development programs prior to bringing a preclinical candidate to a clinical trial and prior to the initiation of a clinical trial program. Data from preclinical analytics could assist in the defining the patient populations that would be best suited for a specific clinical trial (disease target and drug).

At the outset of the clinical trial, say trial phase 1, sponsors and sites will have the ability on the mobile app to define biomarkers and for the app to autonomously identify biomarkers that should be of concern to the trial site and sponsor. However, this may also occur during the other phases of clinical trials e.g., phase 1, phase 2, phase 3, etc. Before any ongoing trial data flows from clinicians and patients to the cloud-based machine learning model, predictions of biomarkers of concern are initialized. As data is input into the mobile application via clinicians and patients, some of that data may flow to the cloud-based machine learning model and the predictions of biomarkers of concern are iteratively updated as new information becomes available. It is important to remember that both explicit (provided by sponsors or clinicians) and implicit (machine learned) biomarkers of concern are considered by the cloud-based machine learning model.

Nearly all functions provided to sponsors and clinicians via the mobile app, are also afforded and used by the machine learning model. For example, the ability to flag a biomarker or individual for concern. The concern i.e., flag may also vary by significance, such that a sponsor or clinician may flag an increasing BNP level (which can indicate potential SAEs and AEs which could cause death) of one or more patients with a red flag, or indicate to the system that one or more patients have dry mouth with a yellow flag. The flag severity may inform the machine model to hold more weight to one biomarker over another. Biomarkers of concern autonomously discovered by the machine learning model may use another ranking system such as a numerical score or edge weights in a neural network. Regarding the flagging of biomarker, the system 4100 may also create a summary of flagged patients at a trial site that may require additional or more frequent testing due to changing biomarker values that indicate a potential health risk.

Should the cloud-based machine learning model make a decision that at least one of the patients is at risk of a SAE or AE, then other patients may be analyzed for the same biomarker. Patients displaying SAEs and AEs may be analyzed by machine learning to uncover a common biomarker, such that an alert, message, or some form of notification may be sent via the app, email, or other communication to alert sponsors and clinicians of the at-risk cohort such that medical intervention may be executed in a timely manner. The results of such a function means clinical trials, and the expense and resources of them may be salvaged from potential failure due to unacceptable losses, not to mention the avoidance of loss-of-human-life. Specifically restating the above, the combination of edge- and cloud-machine learning give sponsors and clinicians the ability to proactively respond to patient issues, before a patient experiences a SAE or dies and a clinical trial has to be stopped. Biomarkers of interest or concern may have reports generated by the clinical trials module which is provided to sponsors and clinicians via the edge device mobile app. Furthermore, as events unfold and information is provided in near real-time to sponsors 4140, sponsors 4140 are afforded expeditious reporting to regulatory agencies 4150 such as the FDA in the United States.

During ongoing trials, sponsors 4140 can identify and update additional endpoints, biomarkers, etc., that could be considered as part of a trial by incorporating them into their mobile application and have them pushed to the edge machine learning mobile apps at their clinical trial sites. Furthermore, sponsor and trial site edge machine learning apps may be updated with relevant longitudinal patient data results from Phase 1 trials to the Phase 2 trials, then to the Phase 3 trials.

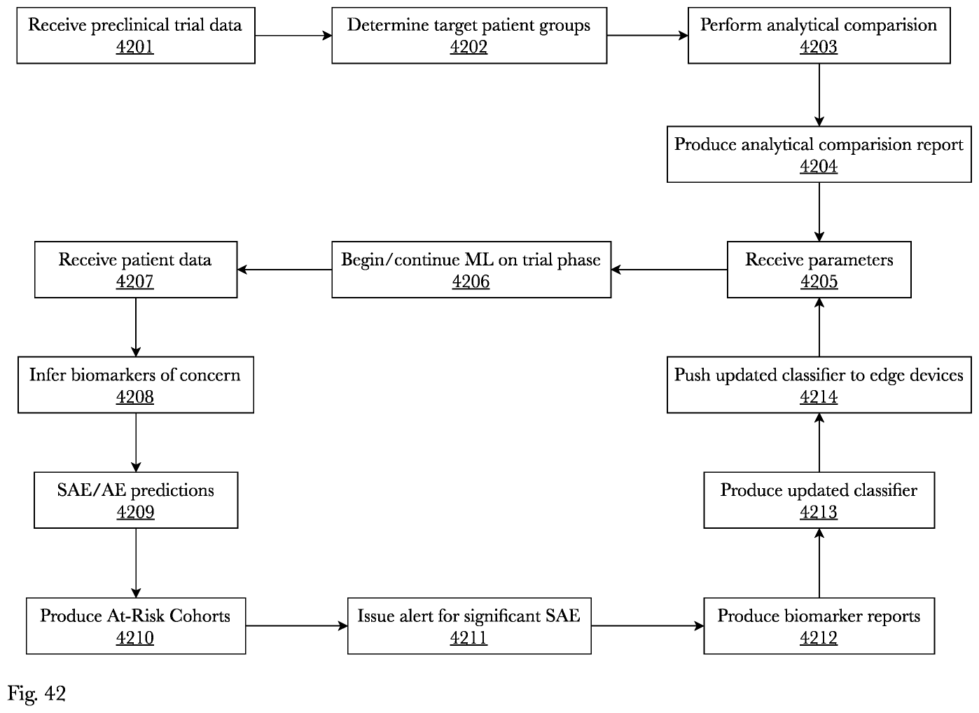

FIG. 42 is a flow diagram illustrating an exemplary method for clinical trial analysis using a pharmaceutical research system. The order in which these steps take place are not limiting to the invention. Especially in regards to the iterative cycle of elements 4205 through 4214. This method diagram is merely exemplary and is intended to provide one embodiment of the system and method disclosed herein. For example, producing an updated classifier 4213 does not necessarily need to happen after issuing an alert of a significant SAE 4211. Again, the order of these steps are only one example of many anticipated embodiments.

According to one embodiment, preclinical trial data is received 4201 and processed by machine learning in order to perform 4203 and output an analytical comparison 4204 to past and current trials 4203, and a predictive determination of the best patient target groups for the clinical trial following the preclinical trial 4202. Designing the clinical trial model is performed typically by sponsors and sometimes clinicians who will input a series of parameters 4205 such as trial endpoints and biomarkers of interest. As the trial begins (or continues as part of an iterative process) 4206, (optional) patient data from edge devices (e.g., heart rate and glucose monitors, etc.) is received 4207 and is agglomerated with the existing and incoming data to assist the machine learning in inferring biomarkers of interest or concern 4208 and subsequently making predictions about SAEs and AEs 4209 from those inferences and data. Identified SAEs and the patient's associated with them are identified within an at-risk cohort 4210 which is provided to sponsors and clinicians along with a generated report 4212. If the SAE or biomarker is significant, alerts and notifications may be automatically issued 4211. Throughout the trial, as the machine learning improves and updates its model, so does it update the model used by edge devices 4213. The machine learning is able to push updated models (e.g., a classifier as one example) to the edge devices via the application server 4214.

### Detailed Description of Exemplary Aspects

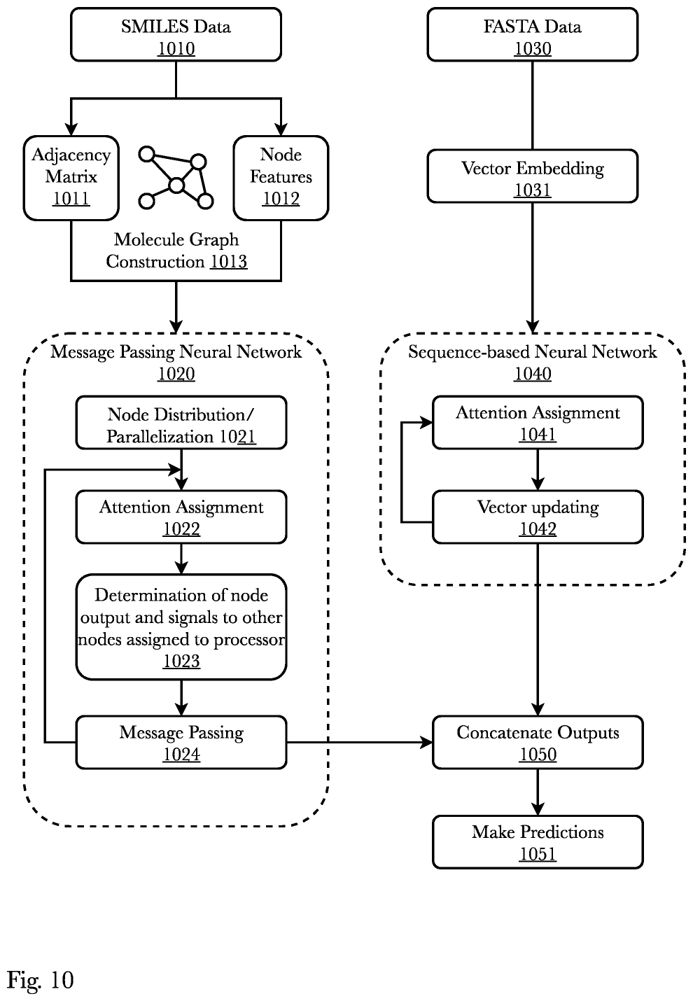

FIG. 10 is a diagram illustrating an exemplary architecture for prediction of molecule bioactivity using concatenation of outputs from a graph-based neural network which analyzes molecules and their known or suspected bioactivities with proteins and a sequence-based neural network which analyzes protein segments and their known or suspected bioactivities with molecules. In this architecture, in a first neural network processing stream, SMILES data 1010 for a plurality of molecules is transformed at a molecule graph construction stage 1013 into a graph-based representation wherein each molecule is represented as a graph comprising nodes and edges, wherein each node represents an atom and each edge represents a connection between atoms of the molecule. Each node represents the atom as node features comprising an atom type and a number of bonds available for that atom. The node features are represented as a node features matrix 1012. The molecule, then, is represented as nodes (atoms) connected by edges (bonds), and is specified as an adjacency matrix 1011 showing which nodes (atoms) are connected to which other nodes (atoms).

At the training stage, the adjacency matrices 1011 and node features matrices 1012 for many molecules are input into the MPNN 1020 along with vector representations of known or suspected bioactivity interactions of each molecule with certain proteins. Based on the training data, the MPNN 1020 learns the characteristics of molecules and proteins that allow interactions and what the bioactivity associated with those interactions is. At the analysis stage, a target molecule is input into the MPNN 1020, and the output of the MPNN 1020 is a vector representation of that molecule's likely interactions with proteins and the likely bioactivity of those interactions.

Once the molecule graph construction 1013 is completed, the node features matrices 1012 and adjacency matrices 1011 are passed to a message passing neural network (MPNN) 1020, wherein the processing is parallelized by distributing groups 1021 nodes of the graph amongst a plurality of processors (or threads) for processing. Each processor (or thread) performs attention assignment 1022 on each node, increasing or decreasing the strength of its relationships with other nodes, and outputs of the node and signals to other neighboring nodes 1023 (i.e., nodes connected by edges) based on those attention assignments are determined. Messages are passed 1024 between neighboring nodes based on the outputs and signals, and each node is updated with the information passed to it. Messages can be passed between processors and/or threads as necessary to update all nodes. In some embodiments, this message passing (also called aggregation) process is accomplished by performing matrix multiplication of the array of node states by the adjacency matrix to sum the value of all neighbors or divide each column in the matrix by the sum of that column to get the mean of neighboring node states. This process may be repeated an arbitrary number of times. Once processing by the MPNN is complete, its results are sent for concatenation 1050 with the results from a second neural network, in this case a long short term memory neural network 1040 which analyzes protein structure.

In a second processing stream, FASTA data 1030 is converted to high-dimensional vectors 1031 representing the amino acid structure of proteins. The vectors are processed by a long short term memory (LSTM) neural network 1040 which performs one or more iterations of attention assignment 1041 and vector updating 1042. The attention assignment 1041 of the LSTM 1040 operates in the same way as that of the MPNN 1020, although the coding implementation will be different. At the vector updating stage 1042, the vectors comprising each cell of the LSTM 1040 are updated based on the attention assignment 1041. This process may be repeated an arbitrary number of times. Once processing by the LSTM 1040 is complete, its results are sent for concatenation 1050 with the results from the first processing stream, in this case the MPNN 1020.

Concatenation of the outputs 1050 from two different types of neural networks (here an MPNN 1020 and an LSTM 1040) determines which molecule structures and protein structures are compatible, allowing for prediction of bioactivity 1051 based on known or suspected similarities with other molecules and proteins.

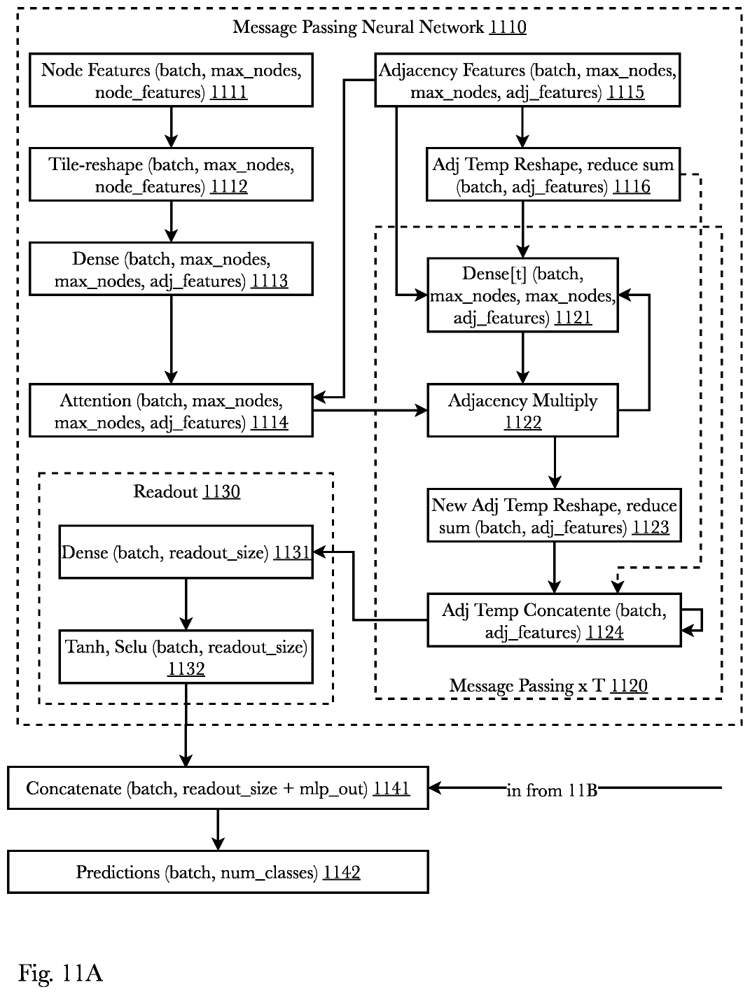

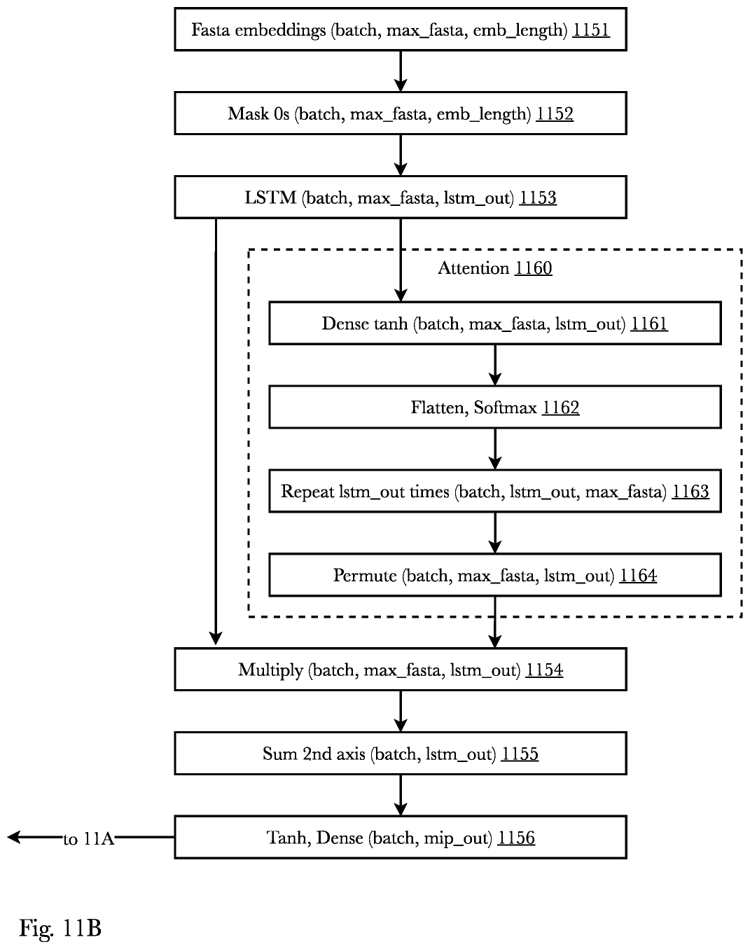

FIGS. 11A and 11B illustrate an exemplary implementation of the architecture for prediction of molecule bioactivity using concatenation of outputs from a graph-based neural network which analyzes molecule structure and a sequence-based neural network which analyzes protein structure. In this example, details regarding a particular implementation of the general architecture shown in FIG. 10 are described.

As shown in FIG. 11A, node features 1111 are received for processing. A reshaping process 1112 may be performed which to conform the dimensionality of the inputs to the dimensionality required for processing by the MPNN. A dense function 1113 is performed to map each node in the previous layer of the neural network to every node in the next layer. Attention is then assigned 1114 using the adjacency matrix contained in the node. The adjacency features (the adjacency matrix) 1115 are simultaneously reshaped 1116 to conform the dimensionality of the inputs to the dimensionality required for processing by the MPNN.

At this stage, a message passing operation 1120 is performed, comprising the steps of performing a dense function 1121 (used only on the first message pass) to map each node in the previous layer of the neural network to every node in the next layer, matrix multiplication of the adjacencies 1122, reshaping of the new adjacencies 1123, and where the message passing operation has been parallelized among multiple processors or threads, concatenating the outputs of the various processors or threads 1124.

Subsequently, a readout operation 1130 is performed comprising performance of a dense function 1131 and implementation of an activation function 1132 such as tanh, selu, etc. to normalize the outputs to a certain range. In this embodiment, the readout operation 1130 is performed only at the first message pass of the MPNN 1110.

As shown in FIG. 11B, FASTA data is converted to high-dimensional vectors 1151, which may then be masked 1152 to conform the vectors to the fixed input length required by the LSTM 1153. The LSTM 1153 then processes the vectors using an attention mechanism 1160 comprising the steps of performing a dense function 1161 to map each node in the previous layer of the neural network to every node in the next layer, performing a softmax function 1162 to assign probabilities to each node just before the output layer. The process is repeated a number of times which may be configured by a parameter 1163. Where permutation invariance is an issue (i.e., where changes in the order of inputs yield changes in the outputs), permutations may be applied to the inputs 1164 to ensure that differences in outputs due to differences in inputs are incorporated.

After attention has been assigned 1160, the vectors in the cells of the LSTM 1153 are multiplied 1154, summed 1155, and a dense function 1156 is again applied to map each node in the previous layer of the neural network to every node in the next layer, and the outputs of the LSTM 1153 are sent for concatenation 1141 with the outputs of the MPNN 1110, after which predictions can be made 1142.

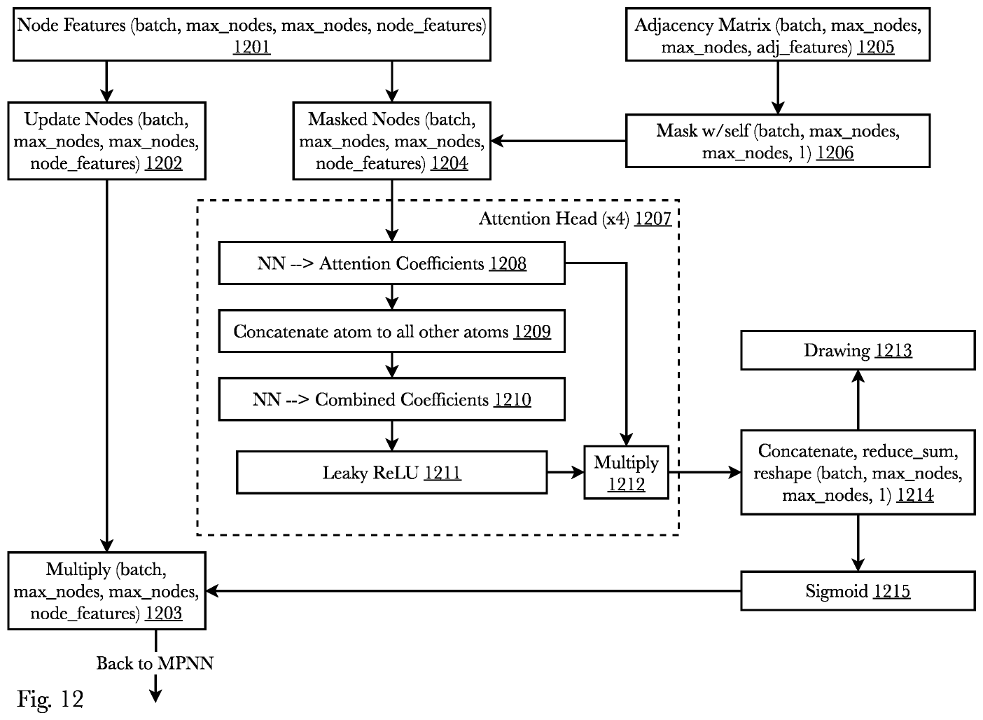

FIG. 12 illustrates an exemplary implementation of an attention assignment aspect of an architecture for prediction of molecule bioactivity using concatenation of outputs from a graph-based neural network which analyzes molecule structure and a sequence-based neural network which analyzes protein structure. This is an exemplary implementation of attention and may not be representative of a preferred embodiment. In this example, details regarding a particular implementation of the attention assignment blocks shown in FIG. 10 are described. The particular implementation of this example involves a multi-head attention mechanism.

As node features 1201 are received for processing, they are updated 1202 and sent for later multiplication 1203 with the outputs of the multiple attention heads 1207. Simultaneously, the nodes are masked 1204 to conform their lengths to a fixed input length required by the attention heads 1207. The adjacency matrix 1205 associated with (or contained in) in each node is also masked 1206 to conform it to a fixed length and sent along with the node features to the multi-head attention mechanism 1207.

The multi-head attention mechanism 1207 comprises the steps of assigning attention coefficients 1208, concatenating all atoms to all other atoms 1209 (as represented in the adjacency matrix), combining the coefficients 1210, performing a Leaky ReLU 1211 function to assign probabilities to each node just before the output layer, and performing matrix multiplication 1212 on the resulting matrices.

The outputs of the multi-head attention mechanism 1207 are then concatenated 1214, and optionally sent to a drawing program for display of the outputs in graphical form 1213. A sigmoid function 1215 is performed on the concatenated outputs 1214 to normalize the outputs to a certain range. The updated node features 1202 are then multiplied 1203 with the outputs of the multi-head attention mechanism 1207, and sent back to the MPNN.

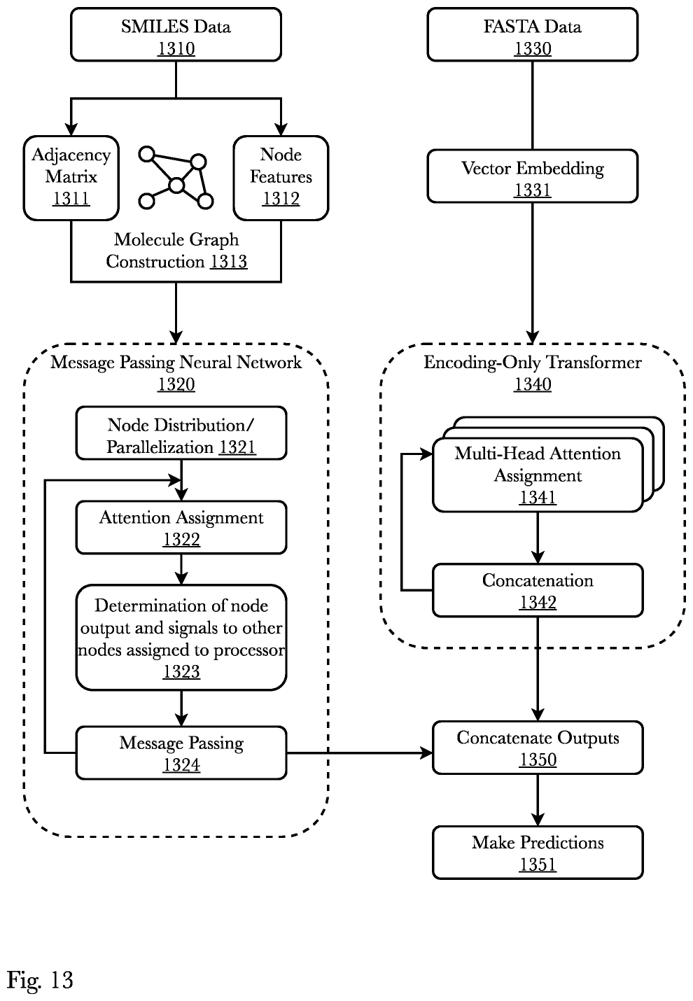

FIG. 13 is a diagram illustrating an exemplary architecture for prediction of molecule bioactivity using concatenation of outputs from a graph-based neural network which analyzes molecules and their known or suspected bioactivities with proteins and a sequence-based neural network which analyzes protein segments and their known or suspected bioactivities with molecules. In this architecture, in a first neural network processing stream, SMILES data 1310 for a plurality of molecules is transformed at a molecule graph construction stage 1313 into a graph-based representation wherein each molecule is represented as a graph comprising nodes and edges, wherein each node represents an atom and each edge represents a connection between atoms of the molecule. Each node represents the atom as node features comprising an atom type and a number of bonds available for that atom. The node features are represented as a node features matrix 1312. The molecule, then, is represented as nodes (atoms) connected by edges (bonds), and is specified as an adjacency matrix 1311 showing which nodes (atoms) are connected to which other nodes (atoms).

At the training stage, the adjacency matrices 1311 and node features matrices 1312 for many molecules are input into the MPNN 1320 along with vector representations of known or suspected bioactivity interactions of each molecule with certain proteins. Based on the training data, the MPNN 1320 learns the characteristics of molecules and proteins that allow interactions and what the bioactivity associated with those interactions is. At the analysis stage, a target molecule is input into the MPNN 1320, and the output of the MPNN 1320 is a vector representation of that molecule's likely interactions with proteins and the likely bioactivity of those interactions.

Once the molecule graph construction 1013 is completed, the node features matrices 1012 and adjacency matrices 1011 are passed to a message passing neural network (MPNN) 1020, wherein the processing is parallelized by distributing groups 1321 nodes of the graph amongst a plurality of processors (or threads) for processing. Each processor (or thread) performs attention assignment 1322 on each node, increasing or decreasing the strength of its relationships with other nodes, and outputs of the node and signals to other neighboring nodes 1323 (i.e., nodes connected by edges) based on those attention assignments are determined. Messages are passed between neighboring nodes based on the outputs and signals, and each node is updated with the information passed to it. Messages can be passed between 1324 processors and/or threads as necessary to update all nodes. In some embodiments, this message passing (also called aggregation) process is accomplished by performing matrix multiplication of the array of node states by the adjacency matrix to sum the value of all neighbors or divide each column in the matrix by the sum of that column to get the mean of neighboring node states. This process may be repeated an arbitrary number of times. Once processing by the MPNN is complete, its results are sent for concatenation 1350 with the results from a second machine learning algorithm, in this case an encoding-only transformer 1340.

In a second processing stream, FASTA data 1330 is converted to high-dimensional vectors 1331 representing the chemical structure of molecules. The vectors are processed by an encoding-only transformer 1340 which performs one or more iterations of multi-head attention assignment 1341 and concatenation 1342. Once processing by the encoding-only transformer 1340 is complete, its results are sent for concatenation 1350 with the results from the neural network, in this case the MPNN 1320.

Concatenation of the outputs 1350 from two different types of neural networks (here an MPNN 1320 and an LSTM 1340) determines which molecule structures and protein structures are compatible, allowing for prediction of bioactivity 1351 based the information learned by the neural networks from the training data.

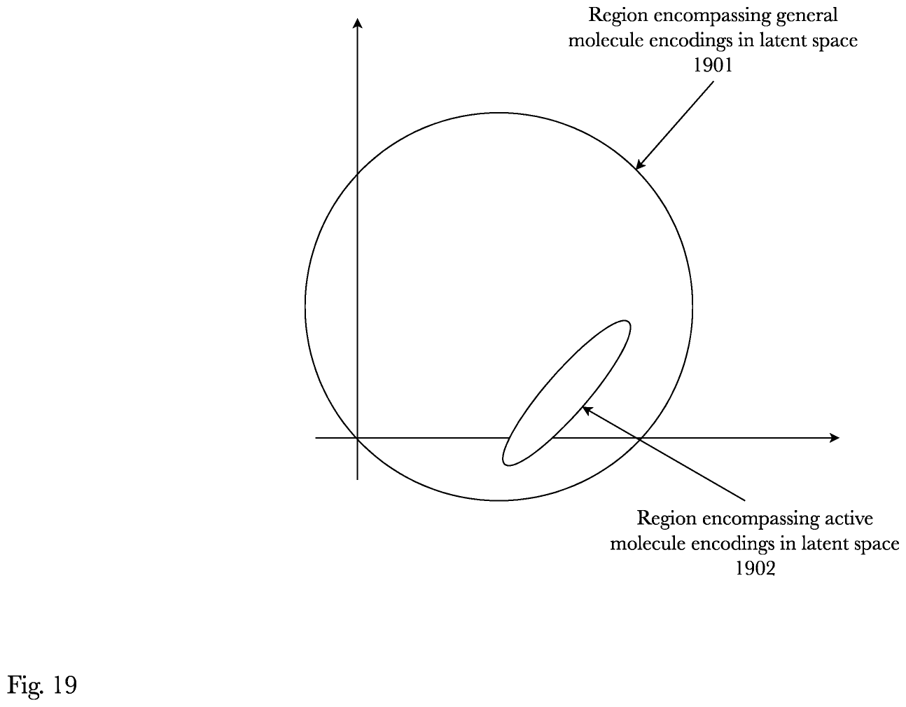

FIG. 19 is a diagram illustrating molecule encodings in latent space 1901. Once a model is trained that achieves a desirable reconstruction accuracy, a pipeline uses the model to generate molecules similar to a target dataset. Evaluating the generated molecules for chemical validity is performed using defined metrics to compare the generated data and to gauge whether the generation method is performing well. There are a few ways to compare how well the generation process works. When attempting to reconstruct the same molecule, the models sometimes produce molecules that are chemically impossible. It is therefore informative to compare the validity ratio of the generated molecules to the validity ratio of the reconstructed molecules of the active dataset. Ideally, the ratio is similar. If, on the other hand, the validity of the generated data is lower, it might mean that: (a) the exploration method of the latent space is not suitable—the explored space goes beyond the chemically meaningful regions; (b) the latent space representation is not smooth enough. A second method is by using molecular weight. The generated molecules are expected to have a similar molecular weight distribution to the active samples—a discrepancy would signal problems similar to those above. Lastly, chemical similarity. Computing and comparing the chemical similarity coefficients to estimate the molecular similarity of the generated and active molecules. This similarity should match the similarity of the active compounds amongst one another. These metrics can be used as a simple check validity (i.e., to see if the generated molecules “make sense”). Validity checking is particularly important in cases where certain properties are imposed, such as log P or molecular weight, to the generated molecules, as this is done by modifying the elements in the latent space, and allow the system to find the viable ranges of these parameters by finding where the above metrics start to deteriorate.

New molecules are generated by estimating a distribution of latent space 1902 that the active molecules are embedded into, then sampling from this distribution 1902 and running the samples through a decoder to recover new molecules. The distribution is approximated by a multivariate Gaussian, with mean and covariance matrices computed from the latent representations of the active molecules.

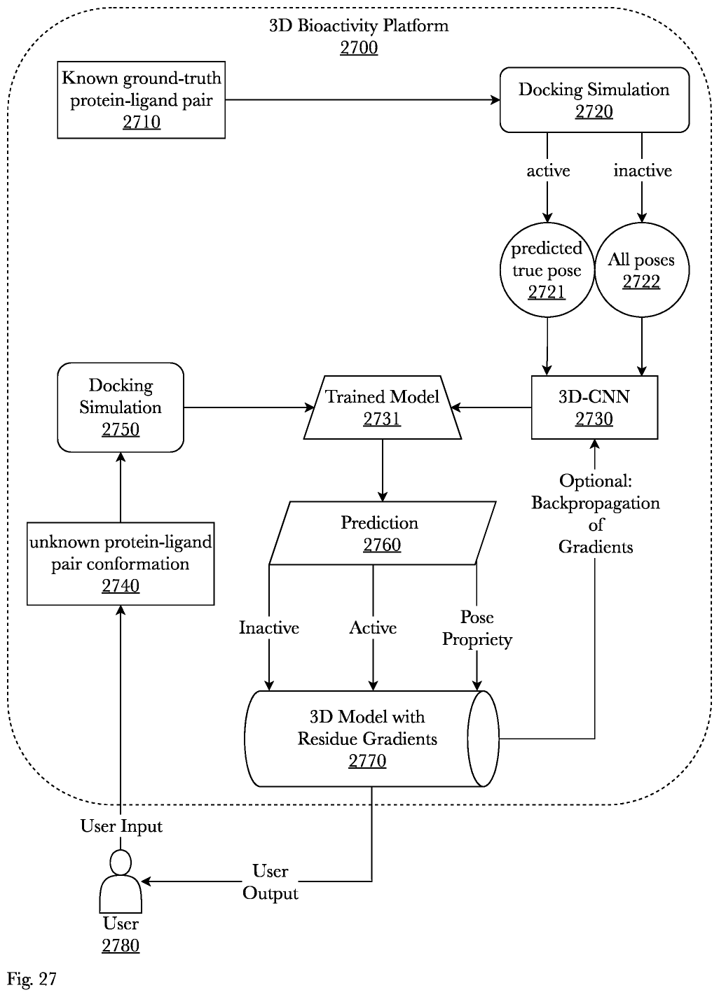

FIG. 27 is a block diagram of an exemplary model architecture for a 3D Bioactivity platform 2700. The model architecture used is a three-dimensional convolutional neural network (3D-CNN) 2730. Convolutional Neural Networks 2730 are widely used on tasks such as image classification. They are multi-layer perceptrons that are regularized in such a way as to take advantage of the translational invariance of the content of pictures (e.g., a gavel is a gavel whether it is in the center or corner of an image). In a convolutional layer, each output neuron is not connected to all the input neurons, but to a spatially-localized subset. CNN architectures operate analogously in higher-dimensional spaces. Docking simulations 2720/2750 take as input the ligand and protein molecules 2710/2740 and their three-dimensional structures. Docking 2720 assigns scores to each pose 2721/2722 to be used in the model 2731 depending on the embodiment. Some embodiments may use all poses, whereas other embodiments use only the highest scored pose for active molecules and all poses for inactive molecules. After docking simulations 2720/2750 have been completed, molecules are voxelated and are used as the model 2731 input, which are used to train the model 2731 to predict 2760 or classify these voxelated representations into active/inactive and pose propriety categories.

In reality, the observed bioactivity of a ligand is not due to a single pose within the binding site, but due to the contributions from a number of possible poses. According to one embodiment, the population of a given pose is given as:

$${W}_{b} = {e}^{\frac{- E}{k T}}$$

where E, k and T correspond to the free energy of binding, Boltzmann's constant, and the temperature, respectively. An estimate of E from the Force Field can be determined, and subsequently the loss may be defined as:

$$L = \frac{{Σ}_{poses} ( {W}_{b} * {( Model ( pose ) - True\_affinity )}^{2} )}{{Σ}_{poses} ( {W}_{b} )}$$

This loss function corresponds to interpreting E not as the true free energy of binding, but instead as the probability of a pose being the “true” pose. This method allows for superimposing the probability-weighted atom density grids, which speeds computation up enormously. The loss function above is merely exemplary and modifications to the loss function above are anticipated.

According to an aspect of various embodiments, an additional ‘Pose Score’ output node to the CNN is improvised. 3D-CNNs 2730 comprise an additional output node that is trained on classifying the input poses as being “low” root-mean-square deviation (RMSD) (<2 Angstrom RMSD vs. crystal structure) and “high” RMSD (>2 Angstrom RMSD vs. crystal structure). This predicted classification is used to modulate the binding-affinity loss as follows: Affinity prediction is trained using an L2-like pseudo-Huber loss that is hinged when evaluating high RMSD poses. That is, the model is penalized for predicting both a too low and too high affinity of a low RMSD pose, but only penalized for predicting too high an affinity for a high RMSD pose. Since the PDB dataset used comprises crystal structures for each available datapoint, it is possible to generate corresponding classification labels into high/low RSMD poses for each docked complex. Two aspects of various embodiments are therefore anticipated. The first aspect comprises extracting RMSD labels for datapoints where crystal structures are available and do not contribute any “Pose Score” loss to the remaining items. The second aspect comprises using Boltzmann-averaging of pose predictions. This second aspect has the advantage of not requiring crystal structures of any complexes.

The output 2770 of the model 2731 may combine the separate poses at test-time. Actions taken on the predictions may be selected from one of the actions in the list comprising: Analogous Boltzmann-weighing of the predictions, Averaging of the predictions across all poses, simple predictions only on the best pose, or any combination thereof.

The visualizations 2770 produced by the model 2731 may use methods such as integrated gradients, which require only a single forwards/backwards pass of the models, which is an improvement over the current state of the art. According to various embodiments, integrated gradients, and other gradient visualizations are achieved by computing the voxel saliencies, and coloring a surface/molecule of its properties. If a MaxPool layer is an initial layer of the model 2731, simple smoothing (i.e., halving the resolution of the grid) may correct the visualization from the zero-average voxel-importance.

Other visualizations methods comprise assigning voxel-gradients back to the atoms of the input molecules, which are adapted to propagate whatever importances are computed for each voxel. Importances provide the user with an explanation of which parts of the protein-ligand pair the model 2731 predicts is most strongly bonded. The more important the atom, the higher the number. The number may be represented by one or more colors or shading. The importance reference system described above, i.e., the color-coordinated importances, is only one example of an importance reference system. Other methods such as coloring, shading, numbering, lettering, and the like may be used.

One use of the exemplary 3D bioactivity platform 2700 embodiment disclosed herein comprises a user 2780 that inputs unknown molecule conformations 2740 into the 3D bioactivity platform 2700 and receives back a prediction as to whether the molecule is active or inactive, a pose score (telling the propriety of the pose), and a 3D model complete with gradient representations of the significant residues 2760/2770.

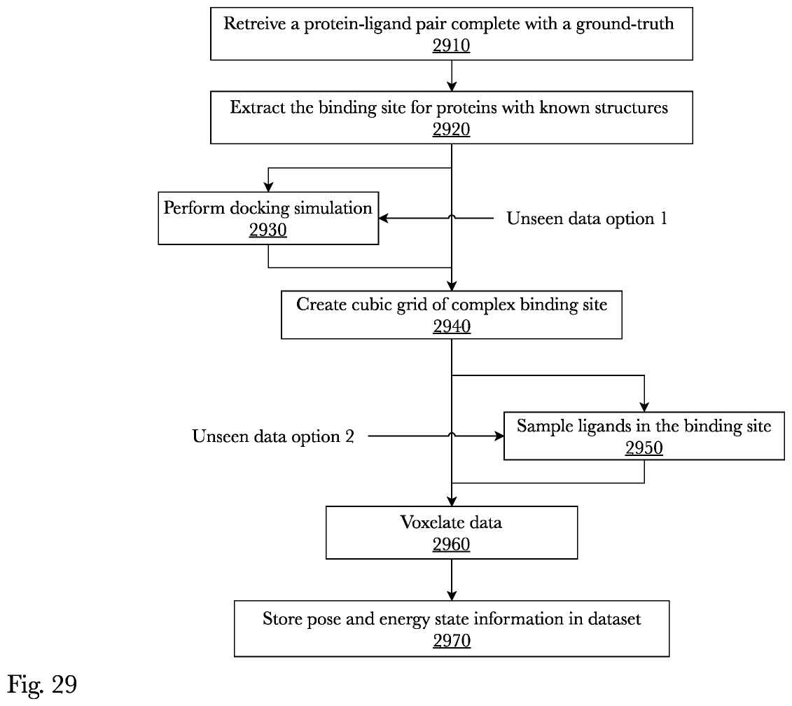

FIG. 29 is a flow diagram illustrating an exemplary method for generating data for use in training a 3D-CNN used by a 3D Bioactivity platform. Training data is generated for the training of the classifier via docking, wherein the method of docking gives the energy states of each protein-ligand pose. The lower the energy state, the stronger the binding affinity. Inputs for the docking mechanism comprise a particular protein-ligand pair and its ground-truth state (i.e., whether it is active or inactive) 2910. On such a pair, the docking simulation is performed and if the pair is labeled as inactive, all data points are kept in the training dataset, if an active label is found as the ground truth state, only the best (lowest energy) pose is kept. According to another embodiment, the top 20 (lowest energy) poses are kept for the training dataset. Further anticipated embodiments acknowledge that any number of poses may be kept for training and the examples contained herein are merely exemplary. According to aspects of various embodiments, simple force-field based optimization of a ligand pose in a binding pocket can substitute for docked poses at reduced computational expense in a binding affinity prediction task without a significant decrease in accuracy. Force-field optimization considers at least one of the constant terms selected from the list of dissociation, inhibition, and half-concentration (IC50) in order to capture the molecular interactions, e.g., hydrogen bonds, hydrophobic bonds, etc. Many databases known in the art may be used to get this information such as the Protin Data Bank (PDB) as one example. In simple terms, docking guides the machine learning (3D-CNN) to realize what poses to keep and to realize what the molecule likely looks like in the pocket.

Prior to featurization, the model input should be a cubic grid centered around the binding site of the complex, the data being the location and atom type of each atom in each the protein and ligand, flagged as to belonging either to the protein or the ligand. This is trivial for complexes with known structures, wherein the binding site is the center of the ligand. For unseen data, two exemplary options are anticipated: generate complexes using docking, or generate complexes by sampling ligand poses.

According to one embodiment, an initial step in dataset creation is to extract the binding sites from all the proteins for which have known structures (this need only be done once ever) 2920. Next, using the aforementioned docking option, complexes are created via docking simulations 2930. However, if the foregoing second option is used, then sampling the ligands in the binding site using the cropped protein structures may be done post-step three for faster data loading 2950. The next step 2940 is to crop to a 24 Angstrom box around the binding-site center (either geometric or center-of-mass). The data is then voxelated 2960 and stored in a dataset 2970. Different box sizes or centering choices is anticipated, however, in one embodiment, the data is voxelated to a certain resolution, e.g., 0.5 Angstrom. This resolution is sensible as it ensures no two atoms occupy the same voxel.

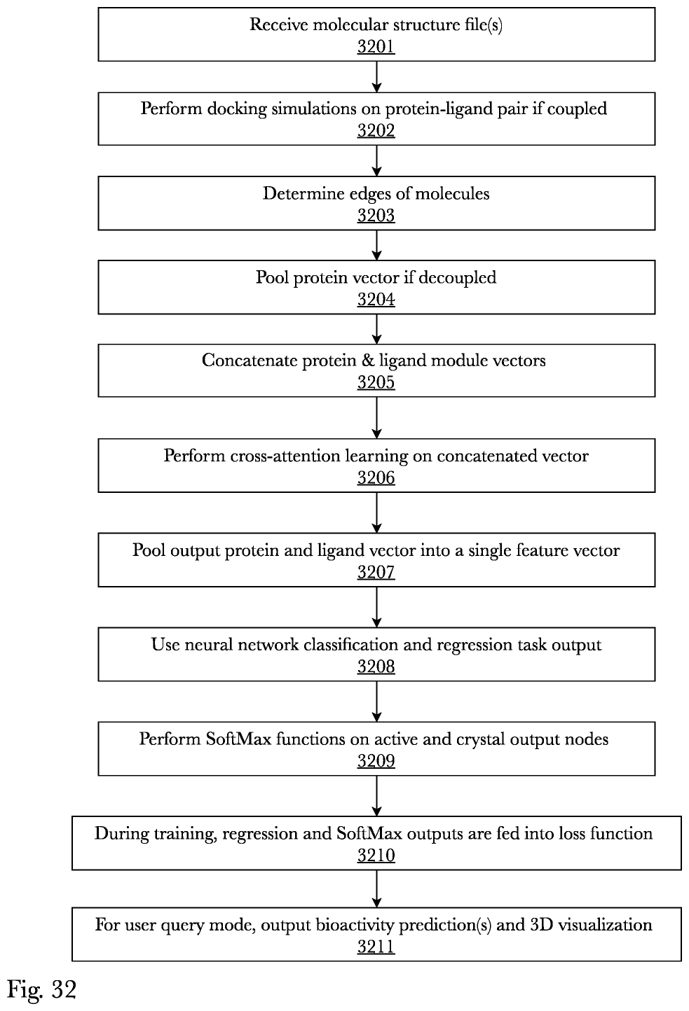

FIG. 32 is a flow diagram illustrating an exemplary method for classifying protein-ligand pairs using a Point-Cloud Bioactivity platform. A first pass through the method illustrated described is to train the model for future queries of user submitted protein-ligand pairs. In such a case, ground-truth protein-ligand pairs (in the form of molecular structure files) 3201 are used in docking simulations 3202 to determine one or more best poses of the molecular conformation. Docking simulations guide the machine learning to realize what poses to keep and to realize what the molecule likely looks like in the pocket. Next, docking simulations output molecular structure files that are used by transformer convolution classifiers to generate edges between the atomic coordinates of the proteins and ligands 3203. If the docking simulations fail, protein atoms of interest are pooled into a single vector 3204 and concatenated with the ligand feature vector 3205, otherwise the protein atoms within a spatial threshold of some amount (according to one embodiment, 4 angstroms) are kept and concatenated with the ligand vector 3205. An attention-restricted (also cross-attention) transformer convolution classifier is used to learn the importances and interactions between the protein-ligand pair 3206. The cross-attention module outputs a feature vector for each atom in each molecule which is then pooled into a single feature vector for each molecule 3207. A neural network—a feed-forward neural network in a preferred embodiment—outputs one to five outputs 3208 depending on the task, which in turn may make up to three predictions. One output is a prediction of the generated protein-ligand pair's active or inactive nature, while another is whether the crystal structure closely matches the crystal structure of the ground-truth pair, typically within 2 angstroms 3209. The regression is used to determine bioactivity which may be a numerical value generated by a regression task and based off one or more bioactivity metrics such as the pair's inhibition constant, IC_{A} or another metric. During training, outputs are fed into a loss function and optimizer for backpropagation as is typical of machine learning 3210, while also using the crystalline structure similarity prediction to determine the legitimacy of the model's bioactivity prediction.

A second pass through the diagram illustrates the case of employing an already trained point-cloud based bioactivity prediction model as described above. A user may submit a query, in the form of a molecular structure file, or other format which is then turned into a molecular structure file, and receive in return one or more predictions selected from the list of active classification, crystal similarity classification, regression task, combined active/crystal classification, a bioactivity classification, or some combination thereof, as well as a three-dimensional point-cloud based visualization 3211 highlighting importances and saliences of the queried molecule. Further information on the visualization follows in the next figure.

FIG. 33 is a block diagram illustrating an exemplary point-based visualization. This diagram is merely for illustrative purposes and is not to scale, may not reflect actual protein-ligand interactions, and may not accurately reflect the visualizations produced by the point-cloud bioactivity module. In this diagram of a point-based visualization, a protein 3301 and ligand molecule 3302 show directly the importance assigned to protein-ligand interactions represented by size 3308, shading 3303/3304, and dashed lines 3305. The point-based visualization colors atoms 3303/3304 according to the positive/negative-ness of the saliencies obtained through integrated gradients, values of attention coefficients, or any variation thereof, as well as scaling their size 3308 according to the magnitude, as can be seen by the larger atoms. Colored dashed lines 3305-3307 are used to highlight importance attributed to interactions and are typically, but not limited to be always within 4 angstroms of each other.

This visualization is based on a point-cloud model with a transformer convolution architecture. This allows specification of edge features and does not require padding, and is thus the preferred point-based model architecture according to one embodiment. This embodiment performs graph-message-passes with messages computed using attention of neighbors, whilst taking edge features into account. By computing the importance with respect to the protein-ligand edge features (which are embeddings of the distance into a series of sinusoids of various frequencies, analogous to the positional embedding used in transformer models), and considering all protein-ligand atom-pairs within 4 Angstrom of one another to be connected by edges, model attributions to certain interactions may be directly highlighted.

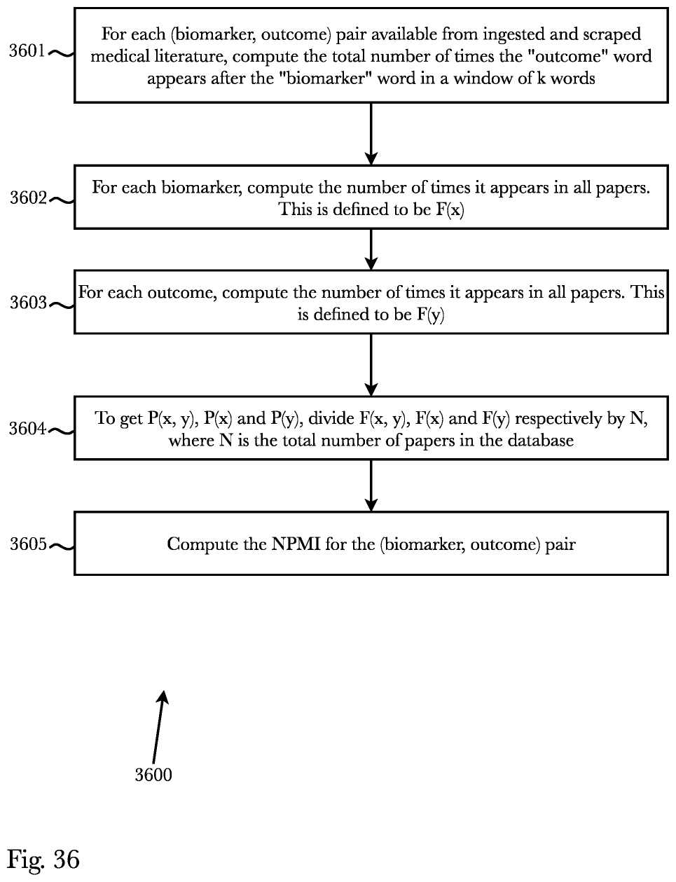

FIG. 36 is a flow diagram illustrating an exemplary method 3600 for calculating the association score for a biomarker-outcome pair, according to one embodiment. The biomarker-outcome prediction and medical literature exploration system xx may compute an association score for a given biomarker-outcome pair. In a preferred embodiment, the association score may be computed using a normalized pointwise mutual information (NPMI) function to measure the co-occurrence of the “biomarker” word and the “outcome” word. Given two points (words) x and y, the pointwise mutual information (PMI) is defined as:

$$PMI ( x , y ) = {log}_{2} \frac{P ( x , y )}{P ( x ) P ( y )}$$

If there is really an association between a biomarker and an outcome, the probability of observing the (biomarker, outcome) pair in one group of words will be much higher than what is expected by chance. The normalized pointwise information is:

$$NPMI ( x , y ) = \frac{PMI ( x , y )}{- {log}_{2} P ( x , y )}$$

Normalizing the PMI function reduces the error that can occur with less frequently occurring words and also produces a bounded answer that is more readily meaningful from an analysis perspective. The calculated association score represents how often, as referenced in medical literature data, a biomarker and a particular outcome occur together in a medically relevant context.

As a first step, the system may for each biomarker pair available from ingested and scraped medical literature, compute the total number of times the “outcome” word appears after the “biomarker” word in a window of k words 3601. Often k is set to five words, but the size of the window may be adjusted both up and down as needed. The total number computed may be defined as the function F(biomarker, outcome) or synonymously F(x, y). A window of words is selected because, oftentimes in medical literature, a biomarker may be connected to an outcome via verbs or phrases that indicate a relationship. The biomarker-outcome pair may be, for example, Albumin-liver disease which may be associated with each other as described by medical literature such as “The best understood mechanism of chronic hypoalbuminemia is the decreased albumin synthesis observed in liver disease”. From that example it can be seen that both the biomarker (Albumin) and outcome (liver disease) are associated with each other, but that the word pair does not necessarily occur consecutively one after the other, therefore it is necessary to create a window of words in which to capture the co-occurrence of the word pair.

Then, for each biomarker, the system computes the number of times it appears in all papers and define this number to be F(x) 3602. The data platform contains over thirty million medical research papers which pass through a natural language processing (NLP) pipeline which extracts relevant information including, but not limited to, proteins, genetic information, diseases, molecules, biomarkers, clinical trials, assays, and biomarkers. Additionally, for each outcome, compute the number of times it appears in all papers and define this number to be F(y) 3603. The next step is to derive P(x, y), P(x) and P(y) by dividing F(x, y), F(x) and F(y) respectively by N, where N is the total number of papers in the data platform database 3604. Once these values have been derived, the system is able to compute the NMPI for the (biomarker, outcome) pair 3605. This value is the association rank between the pair and may be persisted to a database and viewed when making a biomarker query to the EDA 112.

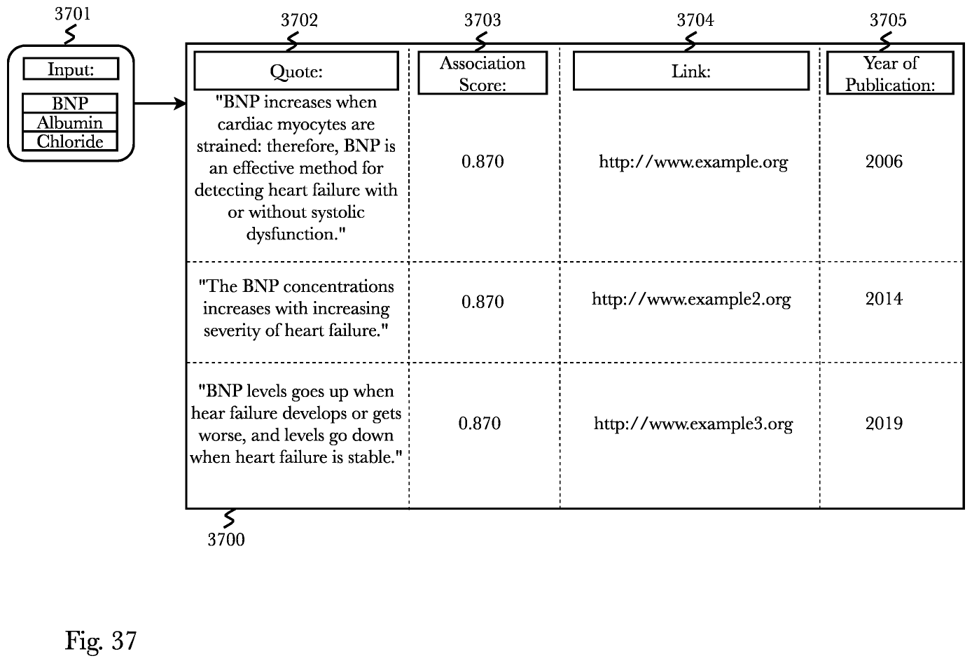

FIG. 37 is a diagram illustrating an exemplary output list 3700 generated from an input list of biomarkers to be measured using the biomarker-outcome prediction and clinical trial exploration system, according to an embodiment. A system user (e.g., pharmaceutical company, contract research organization, etc.) who is interested in running a clinical trial, may input a list 3701 of biomarkers that will be measured during screening or continuously throughout a clinical trial for each patient. The NLP 3501 may be utilized so that for each biomarker the system can return a list of papers that contain associations between that biomarker and side effects, diseases, adverse events, etc.

In this exemplary diagram a biomarker input list 3701 contains three biomarkers of interest: B-type natriuretic peptide (BNP), Albumin, and chloride levels in blood. The biomarker-outcome prediction and medical literature exploration system 3500 returns an output list 3700 of papers that show the associations between the input list biomarkers and some diseases or side effects. In one embodiment, the output list may comprise a quote section 3702 which displays the relevant sentence where the biomarker and outcome are associated, an association score section 3703 which displays the computed association score between an input biomarker and the associated outcome found within the displayed quote, a link section 3704 which provides a clickable web-link where the paper the quote was sourced from can be found in its entirety, and a section that displays the year of publication 3705 of the listed output papers.

In this example diagram, an abridged version of the output list for only the BNP input biomarker is shown, but in practice a biomarker may be associated with hundreds of outcomes and the output list may accordingly span hundreds of papers. Additionally, the output list for each input biomarker would also be displayed, but for simplicity sake the output lists for the other two biomarkers (Albumin and Chloride) have been left out of this exemplary diagram. A pharmaceutical company or research entity may use this system when designing a clinical trial where one or more biomarkers may be measured in order to quickly collate the most relevant and recent information about how each biomarker is related to each outcome.

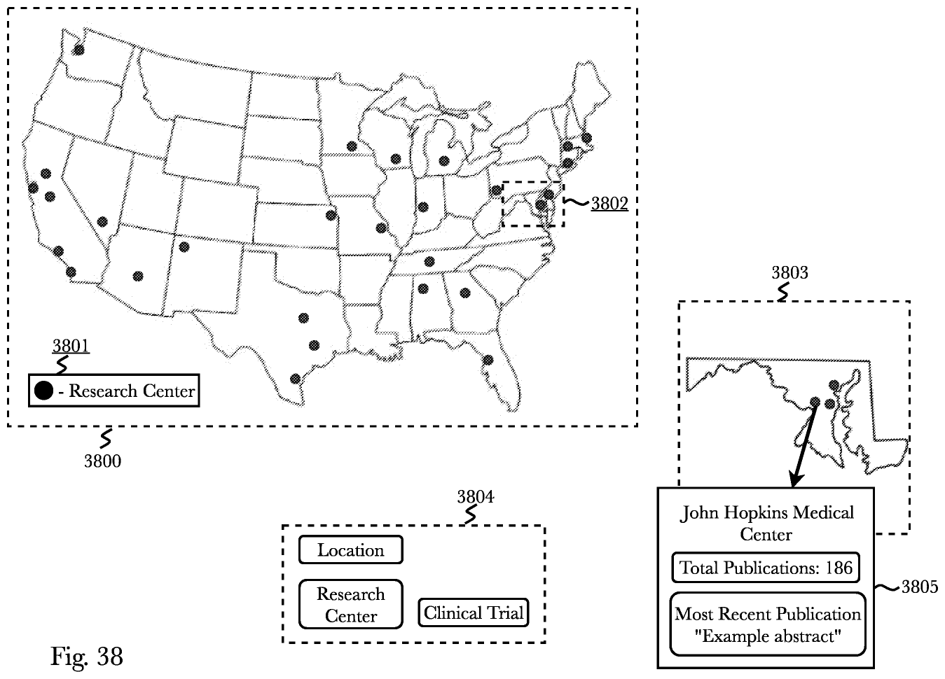

FIG. 38 is a diagram illustrating an exemplary interactive exploration tool in the form of a map 3800 as created by the clinical trial explorer 3506, according to an embodiment. The clinical trial prediction and exploration system 3500 may facilitate exploration of the historical clinical trial, assay, or research paper data by providing a navigable interactive global map of research centers. The clinical trial prediction and exploration system utilizes the geolocation data extracted during data ingestion to provide an interactive map 3800 populated with the locations of research centers and the biomedical literature associated with each research center. Each black dot on the map represents a research center that has published biomedical literature, as described by the legend 3801. The global map is fully navigable in that it allows a user to scroll across the entire map, zoom in or out at a desired location, and click on research centers to view publication originating from there. In a zoomed out view, a user may drag and highlight a subsection 3802 of the graph to provide a zoomed in view 3803 of the selected area.

Additionally, the map may provide one or more of filters 3804 that may allow a user to narrow or broaden the scope of the map. Filters may include, for example, locational filters (e.g., by continent, country, state, city, etc.), research center filters which allow a user to specify which research centers to display, clinical trial filters that allow a user to view clinical trials related to a specific aspect of interest (i.e., disease, biomarker, outcome, adverse event, etc.). A user may hover a computer mouse icon over a research center to cause a research center snapshot 3805 to appear which may provide information including, but not limited to, the research center name, total number of publications produced by the research center, and the abstract of the most recently published paper originating from the research center. Clicking on a research center will cause a new page to be loaded populated with a list of all published papers originating from that research center as well as any available or derived statistics regarding clinical trial data.

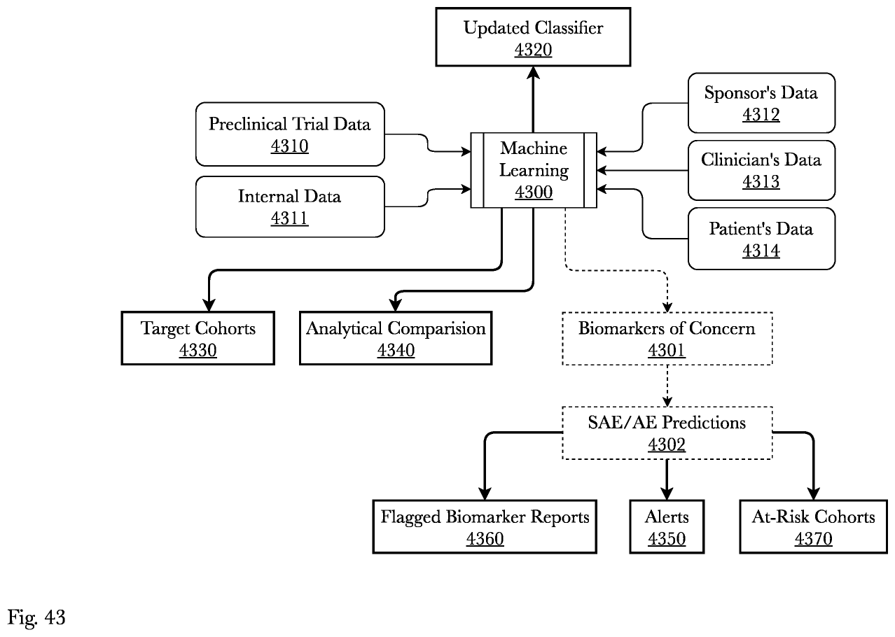

FIG. 43 illustrates the inputs and outputs of a machine learning model for clinical trial analysis. As disclosed in previous figures, the machine learning scheme 4300 comprises both cloud and edge based models. This diagram is an exemplary illustration of the inputs 4310-4314 and outputs 4320-4370 of said machine learning scheme 4300 together as a whole. Preclinical trial data 4310 is used for various operations, some of which comprise providing preclinical comparative analytical comparison of a sponsors target and compound with data for former and current clinical trial sites by variables such as: target, drug, endpoints, SAEs and AEs 4340 and assist in the defining the patient populations that would be best suited for a specific clinical trial (disease target and drug) 4330. Sponsor 4312 and clinician data 4313 may be used for identification of biomarkers, flagging biomarkers (part of 4301), providing patient biometrics (i.e., vital signs, etc.), etc. Patient data 4314 informs the machine learning 4300 about patient biometrics (i.e., vital signs, etc.) and biomarkers and other medical data. Internal data 4311 is any data provided by the various modules in a pharmaceutical research system 4100 as disclosed within this specification in its entirety. The input data 4310-4314 is used to create a machine learning model, which at least identifies biomarkers of concern 4301 and subsequently determines predictions of SAEs and AEs 4302. From those predictions, the machine learning model 4300 may produce reports about flagged biomarkers 4360, groups of patients that are at risk 4370, and alerts 4350 to sponsors and clinicians about biomarkers and predictions of an imperative nature.

FIG. 44 is a diagram of exemplary services provided to edge devices. The services provided by a pharmaceutical research system 4100 may or may not be completed by machine learning, further each separate task may or may not be computed on an edge device and/or a cloud-based device. Machine learning models for the various services illustrated here may be accomplished by one or more multitask learning models, which is where one machine learning model handles more than one task. Or the services may each have a separate machine learning model responsible for computing the task, or any combination thereof. Furthermore, machine learning tasks and the methods thereof may be implemented by the disclosed information regarding the various modules contained within this specification (e.g., knowledge graph, data extraction engine, bioactivity module, clinical trial analyzer, etc.).

Service 1 4401 comprises a feature on a mobile application (or other software platform) that allows sponsors and clinicians to manually define biomarkers of interest. Secondly, one or more machine learning algorithms are tasked with learning to identify biomarkers of interest. The latter task is accomplished by the clinical trials module, but may be assisted by other module such as the ADMET module.

Service 2 4402 comprises a machine learning model that uses indications, values, patterns, and trends in biomarkers to predict SAEs and AEs. Flags, whether determined by the machine learning model or manually by a sponsor or clinician, are cues to the machine learning model to monitor and analyze those specific biomarkers more closely than others. Reports of those biomarkers may be generated either by default, or by the biomarker surpassing some threshold—which may be arbitrarily decided by sponsors and clinicians—and provided via some communication means, typically via the mobile application or autogenerated emails. Alarms, notifications, and other communication means are used to notify sponsors and clinicians of immediate threats to patients or the trial. The threshold of notification may also be chosen by the orchestrators of the trial, as per the mobile application.

Service 3 4403 comprises using the biomarkers and associated patterns to find previously unidentified at-risk patients across all the sites in the trial. This at-risk cohort, generated by machine learning, provides a most expeditious method to intervene at scale to a pending medical emergency. Service 4 4404 comprises using preclinical trial data to determine the patient populations that would be best suited for a specific clinical trial.

Service 5 4405 comprises an iterative process by which there may be a two-way flow of information from the trial site edge devices to the cloud-based models so they can be updated. This includes passive biometric patient data to updated trial parameters from a sponsor to flagged biomarkers from a clinician, as well as pushing updated machine learning models from the cloud to the edge devices. Additionally, as the clinical trial advances through phases, all relevant longitudinal patient data is updated to the edge device mobile applications. Lastly, service 6 4406 comprises providing a preclinical comparative analytical comparison of a sponsor's target and compound with data for former and current clinical trial sites by variables such as: target, drug, endpoints, SAEs and AEs.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the aspects disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific aspects, at least some of the features or functionalities of the various aspects disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some aspects, at least some of the features or functionalities of the various aspects disclosed herein may be implemented in one or more virtualized computing environments (e.g., network computing clouds, virtual machines hosted on one or more physical computing machines, or other appropriate virtual environments).

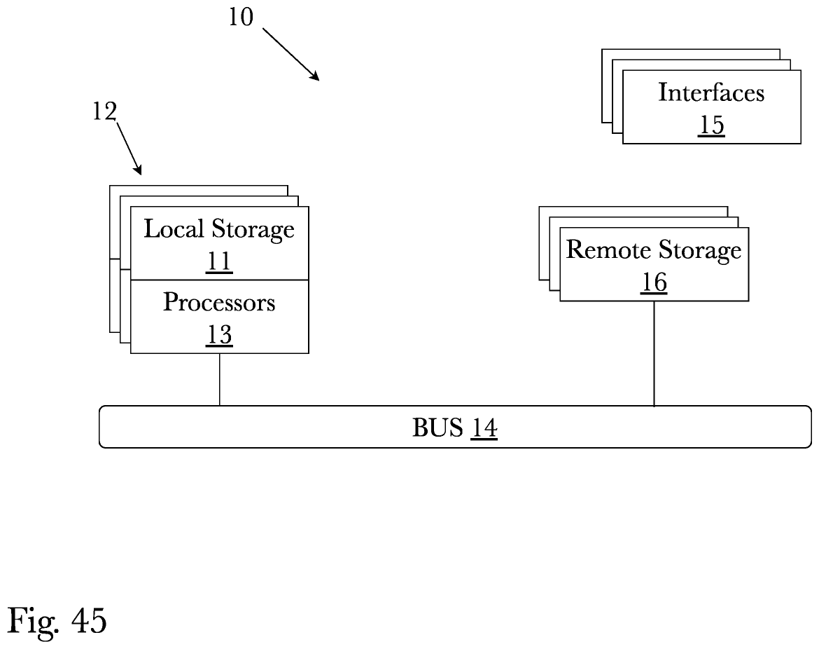

Referring now to FIG. 45, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some aspects, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity AN hardware interfaces) and, in some instances, volatile and/or non-volatile memory (e.g., RAM).

Although the system shown in FIG. 45 illustrates one specific architecture for a computing device 10 for implementing one or more of the aspects described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, a single processor 13 handles communications as well as routing computations, while in other aspects a separate dedicated communications processor may be provided. In various aspects, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the aspects described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device aspects may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably.

Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

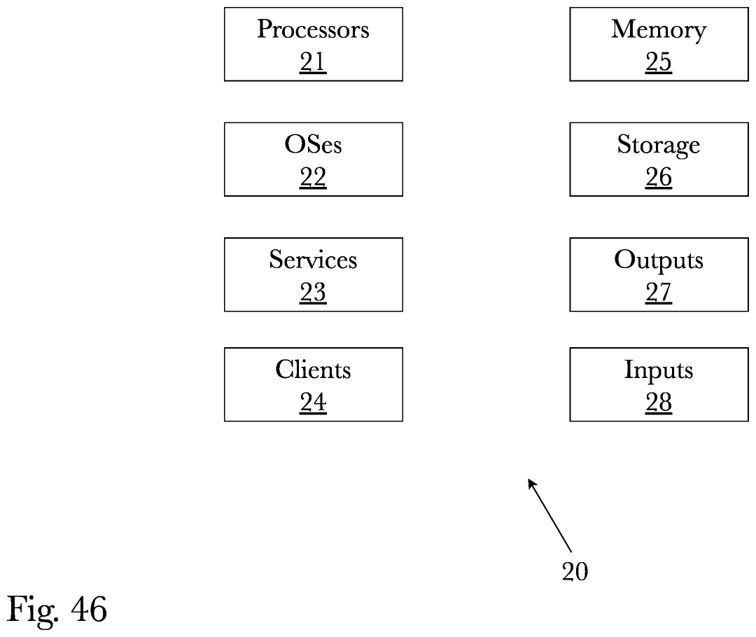

In some aspects, systems may be implemented on a standalone computing system. Referring now to FIG. 46, there is shown a block diagram depicting a typical exemplary architecture of one or more aspects or components thereof on a standalone computing system. Computing device 20 includes processors 21 that may run software that carry out one or more functions or applications of aspects, such as for example a client application 24. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications 24. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 45). Examples of storage devices 26 include flash memory, magnetic hard drive, CD-ROM, and/or the like.

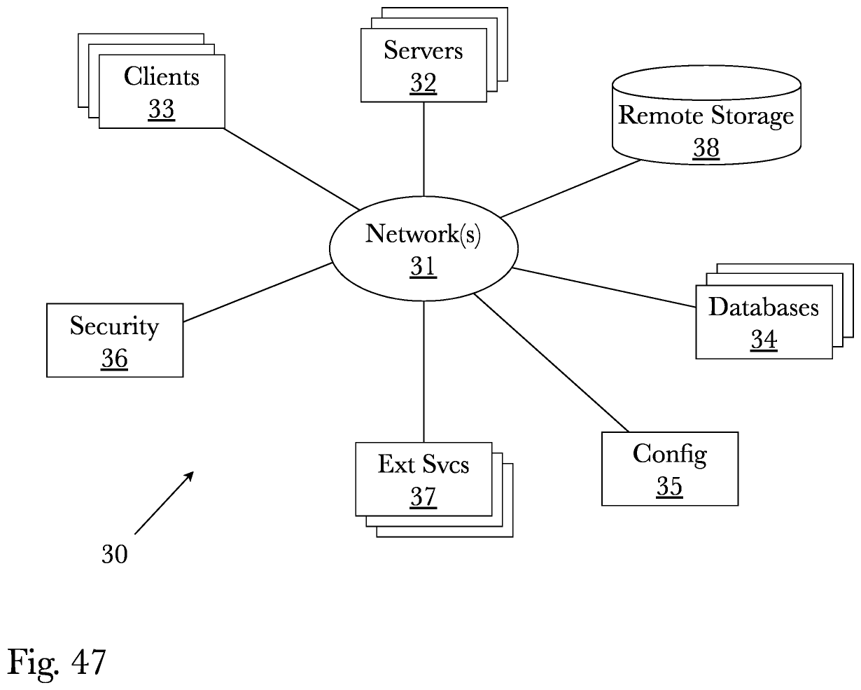

In some aspects, systems may be implemented on a distributed computing network, such as one having any number of clients and/or servers. Referring now to FIG. 47, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 46. In addition, any number of servers 32 may be provided for handling requests received from one or more clients 33. Clients 33 and servers 32 may communicate with one another via one or more electronic networks 31, which may be in various aspects any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some aspects, servers 32 may call external services 37 when needed to obtain additional information, or to refer to additional data concerning a particular call. Communications with external services 37 may take place, for example, via one or more networks 31. In various aspects, external services 37 may comprise web-enabled services or functionality related to or installed on the hardware device itself. For example, in one aspect where client applications 24 are implemented on a smartphone or other electronic device, client applications 24 may obtain information stored in a server system 32 in the cloud or on an external service 37 deployed on one or more of a particular enterprise's or user's premises. In addition to local storage on servers 32, remote storage 38 may be accessible through the network(s) 31.

In some aspects, clients 33 or servers 32 (or both) may make use of one or more specialized services or appliances that may be deployed locally or remotely across one or more networks 31. For example, one or more databases 34 in either local or remote storage 38 may be used or referred to by one or more aspects. It should be understood by one having ordinary skill in the art that databases in storage 34 may be arranged in a wide variety of architectures and using a wide variety of data access and manipulation means. For example, in various aspects one or more databases in storage 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some aspects, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some aspects may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with aspects without limitation, unless a specific security 36 or configuration system 35 or approach is specifically required by the description of any specific aspect.

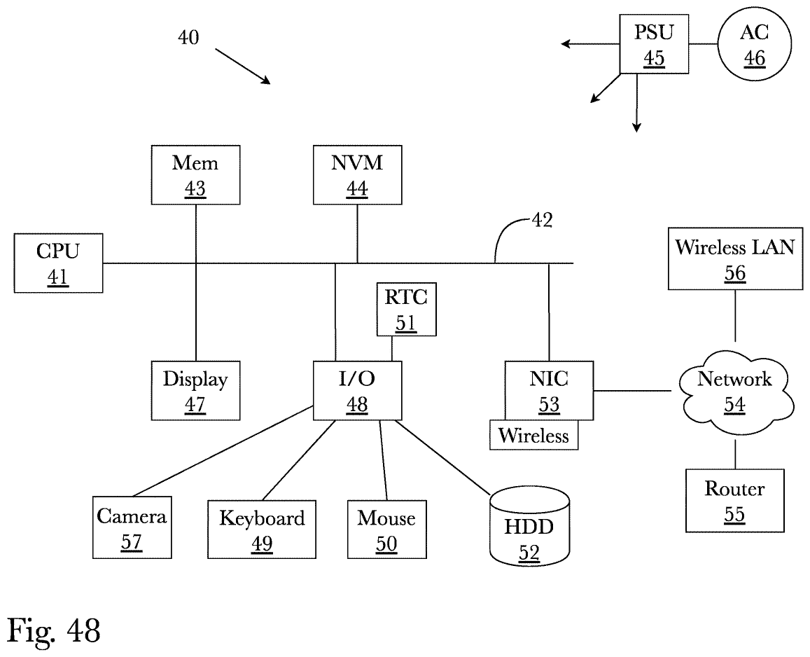

FIG. 48 shows an exemplary overview of a computer system 40 as may be used in any of the various locations throughout the system. It is exemplary of any computer that may execute code to process 3600 data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to peripherals such as a keyboard 49, pointing device 50, hard disk 52, real-time clock 51, a camera 57, and other peripheral devices. NIC 53 connects to network 54, which may be the Internet or a local network, which local network may or may not have connections to the Internet. The system may be connected to other computing devices through the network via a router 55, wireless local area network 56, or any other network connection. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various aspects, functionality for implementing systems or methods of various aspects may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various aspects described above. Accordingly, the present invention is defined by the claims and their equivalents.

1. A system for clinical trial communications, analysis, and predictions comprising:
a software application running on a plurality of edge computing devices, the software application running on each edge computing device being configured to:
receive a machine learning model from a computer server, the machine learning model having been trained to predict an adverse effect of a clinical trial according to clinical trial parameters, the clinical trial parameters comprising a disease and a drug treatment for the disease;
receive patient data from the edge device for a trial patient having the disease, the patient data comprising one or more biomarkers;
process the patient data for the trial patient through the machine learning algorithm to obtain a predicted adverse effect on the trial patient from the drug treatment based on the patient data;
receive an actual outcome from the edge device of drug treatment on the trial patient;
calculate an association score by comparing the predicted adverse effect with the actual outcome; and
send the patient data and the association score to the computer server;
a computer server comprising a memory and a processor;
a clinical trials module, comprising a first plurality of programming instructions stored in the memory and operating on the processor, wherein the first plurality of programming instructions, when operating on the processor, causes the computer server to:
receive the clinical trial parameters;
train the machine learning model to predict an adverse effect of a clinical trial according to the clinical trial parameters;
deploy the machine learning model to the software application;
receive the patient data and the association score from the software application for each of the plurality of edge computing devices;
use the patient data and the association score from the plurality of edge computing devices to retrain the primary machine learning model;
deploy the re-trained machine learning model to the software application;
process the patient data from each of the plurality of edge computing devices through the re-trained machine learning algorithm to predict whether the predicted adverse effect will occur in any trial patient for which patient data has been received;
issue an alert to the software application if the predicted adverse effect is predicted in at least one of the trial patients, the alert comprising identifying information of all the patients at risk of the predicted adverse effect.

2. The system of claim 1, wherein the adverse effect is a serious severe adverse effect, and the alert comprises a warning to stop the drug treatment for one or more of the trial patients.

3. The system of claim 1, wherein the patient data comprises data selected from the group consisting of biometrics, biomarkers, medical history, and vital signs.

4. The system of claim 1, wherein the clinical trial parameters further comprise preclinical trial data.

5. The system of claim 4, wherein the machine learning algorithm trained in part on the preclinical trial data is used to determine target patient groups for a clinical trial.

6.-10. (canceled)

11. A method for clinical trial communications, analysis, and predictions comprising the steps of:
running a software application on a plurality of edge computing devices, the software application running on each edge computing device being configured to:
receive a machine learning model from a computer server, the machine learning model having been trained to predict an adverse effect of a clinical trial according to clinical trial parameters, the clinical trial parameters comprising a disease and a drug treatment for the disease;
receive patient data from the edge device for a trial patient having the disease, the patient data comprising one or more biomarkers;
process the patient data for the trial patient through the machine learning algorithm to obtain a predicted adverse effect on the trial patient from the drug treatment based on the patient data;
receive an actual outcome from the edge device of drug treatment on the trial patient;
calculate an association score by comparing the predicted adverse effect with the actual outcome; and
send the patient data and the association score to the computer server;
using a clinical trials module operating on a computer server comprising a memory and a processor, performing the steps of:
receiving the clinical trial parameters;
training the machine learning model to predict an adverse effect of a clinical trial according to the clinical trial parameters;
deploying the machine learning model to the software application;
receiving the patient data and the association score from the software application for each of the plurality of edge computing devices;
using the patient data and the association score from the plurality of edge computing devices to retrain the primary machine learning model;
deploying the re-trained machine learning model to the software application;
processing the patient data from each of the plurality of edge computing devices through the re-trained machine learning algorithm to predict whether the predicted adverse effect will occur in any trial patient for which patient data has been received; and
issuing an alert to the software application if the predicted adverse effect is predicted in at least one of the trial patients, the alert comprising identifying information of all the patients at risk of the predicted adverse effect.

12. The method of claim 11, wherein the adverse effect is a serious severe adverse effect, and the alert comprises a warning to stop the drug treatment for one or more of the trial patients.

13. The method of claim 11, wherein the patient data comprises data selected from the group consisting of biometrics, biomarkers, medical history, and vital signs.

14. The method of claim 11, wherein the clinical trial parameters further comprise preclinical trial data.

15. The method of claim 14, wherein the machine learning algorithm trained in part on the preclinical trial data is used to determine target patient groups for a clinical trial.

16.-20. (canceled)
